(19) United States
(12) Patent Application Publication
(10) Pub. No.: US 2025/0066804 A1
(43) Pub. Date: Feb. 27, 2025

(54) USE OF TAWOX FOR IMPROVING REGENERATION OF PLANT CELLS

(71) Applicant: BASF Agricultural Solution Seed US LLC, Florham Park, NJ (US)

(72) Inventors: Timothy James GOLDS, Gent (BE); Younousse SAIDI, Gent (BE); Eveline BOSSIER, Gent (BE); Katrien VAN AUDENHOVE, Gent (BE)

(21) Appl. No.: 18/723,327

(22) PCT Filed: Dec. 21, 2022
(86) PCT No.: PCT/US2022/082100

§ 371 Date: Jun. 21, 2024

(30) Foreign Application Priority Data
Dec. 22, 2021 (EP) 21216778.7

(51) Int. Cl. C12N 15/82; C07K 14/415; C12N 9/22; C12N 15/11

(52) U.S. Cl. CPC C12N 15/8213; C07K 14/415; C12N 9/22; C12N 15/111; C12N 15/8207; C12N 2310/20

(57) ABSTRACT

The present invention provides a method for improving regeneration efficiency of a genome edited plant cell, which method comprises the steps of (a) simultaneously introducing an endonuclease designed for a desired genome modification (EDTGM) and a TaWOX, in the form of a protein or in the form of its encoding mRNA, into a plant cell, (b) allowing the endonuclease to modify the genome of said plant cell, and (c) regenerating said plant cell into a plant, thereby allowing the TaWOX protein to increase efficiency of regeneration.

### CROSS-REFERENCE TO RELATED APPLICATION

The present application claims the benefit of priority to European application number 21216778.7, filed Dec. 22, 2021, the entire contents of which is incorporated herein by reference in its entirely.

### REFERENCE TO AN ELECTRONIC SEQUENCE LISTING

The contents of the electronic sequence listing (039621-01027_Sequence-Listing.xml; Size: 46 kilobytes; and Date of Creation: Dec. 9, 2022) are herein incorporated by reference in its entirety.

### FIELD OF THE INVENTION

This invention relates to methods for regenerating plants after targeted modification of the plant genome.

### BACKGROUND

The ever-increasing world population and the dwindling supply of arable land available for agriculture fuels research towards increasing the efficiency of agriculture. Traits of particular economic interest are yield, in terms of both quantity and/or quality. Another important trait for many crops is early vigour and biotic or abiotic stress resistance/tolerance. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed man-kind to modify the germplasm of animals and plants. Genetic engineering of plants entails the isolation and manipulation of genetic material (typically in the form of DNA or RNA) and the subsequent introduction of that genetic material into a plant. Such technology has delivered over the past decades crops or plants having various improved economic, agronomic or horticultural traits (Brookes and Barfoot (2020) GM crops: global socio-economic and environmental impacts 1996-2018 P G Economics Ltd, UK (www.pgeconomics.co.uk). Introduction of desired genetic material into the plant is usually done on a specially adapted T-DNA plasmid which can be introduced via Agrobacterium mediated transformation or, alternatively, with biolistics, electroporation, PEG transfection or a freeze/thaw transformation method (Wise et al. (2006) In: Wang K. (eds) Agrobacterium Protocols. Methods in Molecular Biology, vol 343. Humana Press. https://doi.org/10.1385/1-59745-130-4:43). With each of these methods a T-DNA is introduced that will result in stable or transient expression of the introduced gene of interest. However, T-DNAs are known to frequently integrate as repeats of multiple copies: for example Jorgensen et al. showed that seven out of 11 tested transgenic tomato plants had multiple copies of T-DNA arranged in inverted repeat structures (Jorgensen et al. (1987) Molecular & General Genetics 207, 471-477) and Jupe et al. demonstrated that T-DNA insertion occurred as multiple concatenated full and partial fragments (Jupe et al. (2019) PLoS Genet 15(1): e1007819 (doi.org/10.1371/journal.pgen.1007819). Also gene silencing resulting from T-DNA insertion is described (Vaucheret et al. (1998) Plant Journal 16(6):651-9. doi: 10.1046/j.1365-313x.1998.00337.x. and Schubert et al. (2004) Plant Cell 16, 2561-2572), as well as deletions, inversions and translocations of chromosomal arms (Nacry et al. (1998) Genetics 149(2): 641-650, Tax and Vernon (2001). Plant Physiology 126: 1527-1538. and Gheysen et al. (1991) Genes & Development 5: 287-297). Furthermore, inefficient termination at the left border of the T-DNA may cause integration of vector backbone into the plant genome (Kononov et al. Plant J. 1997, 11, 945-957; Podevin et al. Transgenic Res. 2006, 15, 557-571).

Although the benefits of genetic engineering are beyond doubt, consumer acceptance of GMO (derived) products is not widely accepted, particularly in the field of agriculture. Genome editing has become in the past years a tool of great importance for modifying the genome of an organism in a precise manner. Unlike genetic engineering which heavily relies on random integration of T-DNA in the genome, genome editing allows to introduce precise modifications (such as substitutions, deletions insertions) at a predefined target site in the genome. The technique is known for many years already, but with the advent of CRISPR-Cas systems, genome editing improved in terms of speed, cost, accuracy, and efficiency. By designing a guide RNA that associates with the Cas endonuclease, the complex is guided to the target site in the genome where the Cas protein (or a derivative thereof) can exert its function. After the step of editing, the modified cells—in case of plant cells—need to be regenerated into a complete plant. This regeneration step has often proven to be very difficult: the editing step may cause stress or damage to the cells, cells of some plant species are notoriously difficult or even impossible to regenerate, sometimes only cells of a particular variety or even of a particular genotype can be regenerated.

To improve the efficiency of plant cell regeneration after transformation or genome editing, expression of genes encoding proteins involved in plant growth regu-lation were proven to be useful, in particular so called morphogenic genes (Gordon Kamm et al, Plants 2019, 8, 38; doi:10.3390/plants8020038). Transcription factors like WUSCHEL (WUS) and related WOX, BABY BOOM or LEC1 are known to promote tissue formation or somatic embryogenesis when overexpressed. This property has been used to enhance the efficiency of genome editing in plants. Typically plant cells are transformed with a gene construct encoding a CRISPR-Cas complex and with a gene construct encoding a morphogenic gene (WO 2018/224001, WO 2021/030242, WO 2021/022043). However, this approach has some limitations and drawbacks: the plant cells must be transformable and the expression must be finetuned by carefully choosing a promoter to avoid off-target effects caused by prolonged expression of the CRISPR-Cas complex. In addition, as pointed out above for T-DNA, plasmid DNA may get incorporated in the genome of the plant cell. Furthermore, constitutive (over) expression of morphogenic genes often cause undesired side effects on the plant phenotype (Kyo et al. Plant Biotechnology 35, 23-30 (2018) DOI: 10.5511/plantbiotechnology.18.0126a; Bouchabke-Coussa et al. Plant Cell Rep. 2013 May; 32(5):675-86 doi: 10.1007/s00299-013-1402-9; Kieffer et al. Plant Cell 2006 18: 560-573; Xu et al. Plant Molecular Biology 2005 57: 773-784; Zhao, Ph.D. thesis, Chinese Academy of Sciences, 2015). These problems can be circumvented by introducing ribonucleoproteins (RNP) rather than introducing and overexpressing a nucleic acid encoding a functional CRISPR-Cas system (Kanchiswami Plant Cell Rep 2016 Vol. 35 Issue 7 Pages 1469-74; Liang et al. Nat Commun 2017 8: 14261).

To overcome the disadvantages of the current methods, the inventors have developed a method which allows the targeted modification of the genome of a plant cell and at the same time facilitating regeneration of the plant cell after the targeted modification without the drawbacks of using a transformation step.

### DESCRIPTION

The present invention deals with introducing targeted modifications into the genome of plant cells by genome editing and subsequent regeneration of the modified cells into plants. The genome editing step is performed by delivering into plant cells an endonuclease designed for creating a desired targeted genome modification, which endonuclease can be delivered in the form of protein or its encoding mRNA. It was observed by the inventors that the efficiency of regenerating such genome edited plant cells as well as the speed of regeneration could be increased by simultaneously delivering into the plant cell the endonuclease together with a TaWOX protein or its encoding mRNA, compared to the regeneration efficiency or speed of control plant cells to which only the endonuclease designed for a targeted genome modification (herein abbreviated to EDTGM) was administered but that have otherwise undergone the same treatment. As used herein, the term “TaWOX” may refer to a TaWOX protein, or a Tawox mRNA or to both, as the context requires. The inventors observed that despite the co-introduction of a TaWOX protein or Tawox mRNA together with the EDTGM, the TaWOX protein remained sufficiently long active to be capable of promoting plant regeneration after the step of modifying the genome. The inventors also observed that the genome modification step was not negatively influenced by co-introducing the EDTGM together with the TaWOX protein or Tawox mRNA. The inventors thus designed an improved and DNA-free method of generating plants with a desired modification in their genome, while avoiding the negative effects of transforming a cell with DNA, followed by stable or transient expression of the EDTGM and/or TaWOX.

Therefore, the present invention provides a method for improving regeneration efficiency and/or speed of a genome edited plant cell, which method comprises the steps of (a) simultaneously introducing an EDTGM and a TaWOX, in the form of a protein or in the form of its encoding mRNA, into a plant cell, (b) allowing the endonuclease to modify the genome of said plant cell, and (c) regenerating said plant cell into a plant, thereby allowing the TaWOX protein to increase efficiency of regeneration and/or to shorten the regeneration time, wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

According to a first embodiment, there is provided a method for increasing the efficiency of regenerating a genome edited plant cell into a plant, comprising the steps of:
(a) simultaneously introducing into a plant cell (i) an EDTGM and (ii) a TaWOX, wherein said EDTGM and said TaWOX are introduced as protein;
(b) allowing the EDTGM to modify the genome of said plant cell;
(c) regenerating said plant cell into a plant, thereby allowing the introduced TaWOX to increase efficiency of regeneration;
   wherein the regeneration efficiency is increased compared to the regeneration of corresponding genome-edited plant cells without having said TaWOX introduced.

In another embodiment, there is provided a method for decreasing the regeneration time of a genome edited plant cell into a plant, comprising the steps of:
(a) simultaneously introducing into a plant cell (i) an EDTGM and (ii) a TaWOX, wherein said EDTGM and said TaWOX are introduced as protein;
(b) allowing the EDTGM to modify the genome of said plant cell;
(c) regenerating said plant cell into a plant, thereby allowing the introduced TaWOX to decrease the time of regeneration;
   wherein the regeneration time is decreased compared to the regeneration time of corresponding genome-edited plant cells without having said TaWOX introduced.

Alternatively, step (a) in the above embodiments is performed by simultaneously introducing into a plant cell (i) an EDTGM in the form of a protein and (ii) a mRNA encoding a TaWOX. In another variant, step (a) is performed by simultaneously introducing into a plant cell (i) a mRNA encoding an EDTGM and (ii) a TaWOX protein. In yet another variant, step (a) is performed by simultaneously introducing into a plant cell (i) a mRNA encoding an EDTGM and (ii) a mRNA encoding a TaWOX protein.

In one particular embodiment, step (a) is performed by simultaneously introducing into a plant cell (i) an EDTGM in the form of a protein and (ii) a mRNA encoding a TaWOX and (iii) a TaWOX protein. Preferably, EDTGM in this embodiment is a CRISPR-Cas system in the form of RNP.

The present invention is exemplified by the following non-limiting list of embodiments:

In a first embodiment, the present invention provides a method for improving regeneration efficiency and/or speed of regeneration of one or more genome edited plant cell (s) into a plant, which method comprises the steps of
a) simultaneously introducing an EDTGM protein and a TaWOX protein into a plant cell,
b) allowing the EDTGM protein to modify the genome of said plant cell, and
c) regenerating said plant cell into a plant, thereby allowing the TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a second embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX mRNA into a plant cell,
b) having the EDTGM mRNA and the TaWOX mRNA transcribed in the cell,
c) allowing the transcribed EDTGM protein to modify the genome of said plant cell, and
d) regenerating said plant cell into a plant, thereby allowing the transcribed TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a third embodiment, the present invention provides a method for improving regeneration efficiency and/or speed of regeneration of one or more genome edited plant cell (s) into a plant, which method comprises the steps of
a) simultaneously introducing an EDTGM protein and a TaWOX mRNA into a plant cell,
b) having the TaWOX mRNA transcribed in the cell,
c) allowing the EDTGM protein to modify the genome of said plant cell, and
d) regenerating said plant cell into a plant, thereby allowing the transcribed TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a fourth embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX protein into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a fifth embodiment, the present invention provides a method for improving regeneration efficiency and/or speed of regeneration of one or more genome edited plant cell (s) into a plant, which method comprises the steps of
a) simultaneously introducing an EDTGM protein and a mixture of TaWOX protein and TaWOX mRNA into a plant cell,
b) having the TaWOX mRNA transcribed in the cell,
c) allowing the EDTGM protein to modify the genome of said plant cell, and
d) regenerating said plant cell into a plant, thereby allowing the TaWOX protein (transcribed or introduced) to increase efficiency of regeneration and/or to shorten the regeneration time,
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

The TaWOX protein useful in the methods of the invention and as defined herein, refers to a transcription factor polypeptide comprising a Wuschel-like InterPro family IPR044555 domain, comprising a homeobox domain IPR001356 (such as V10-K76 in SEQ ID NO:14) (InterPro 84.0-11 Feb. 2021), preferably also comprising a WUS-box motif (T-L-[DEQP]-L-F-P-[GITVL]-[GSKNTCVL], SEQ ID NO: 32), and/or an ERF-associated amphiphilic repression (EAR) motif (L-[ED]-L-[RST]-L, SEQ ID NO: 33 and/or SEQ ID NO: 40) (van der Graaff et al., Genome Biology 10:248, 2009). In one preferred embodiment, the TaWOX protein comprises the IPR001356 domain, the WUS-box motif and the EAR motif. Thus, the present invention provides the method above for increasing the efficiency of regenerating a genome edited plant cell into a plant and/or to decrease the regeneration time of a genome edited plant cell into a plant, wherein said TaWOX is a transcription factor comprising a Wuschel-like InterPro family IPR044555 domain, preferably further comprising a WUS-box motif and an ERF-associated amphiphilic repression motif.

Alternatively or additionally, a “TaWOX polypeptide” as defined herein refers to any polypeptide comprising a domain having in increasing order of preference at least 70%, 75%, 80%, 85%, 90%, 95%, 98%, 99% or more amino acid sequence identity to the homeobox domain in SEQ ID NO: 14, represented by

(SEQ ID NO: 29)
VKCGRWNPTAEQVKVLTELFRAGLRTPSTEQIQRISTHLSAF-
GKVESKNVFYWFQNHKARERHHHKK.

Alternatively or additionally, a “TaWOX polypeptide” as defined herein refers to any polypeptide comprising a sequence having in increasing order of preference at least 70%, 75%, 80%, 85%, 90%, 95%, 98%, 99% or more overall amino acid sequence identity to the TaWOX polypeptide as represented by SEQ ID NO: 14. Thus, the present invention provides the methods described above for increasing the efficiency of regenerating a genome edited plant cell into a plant and/or for decreasing the regeneration time, wherein TaWOX is a polypeptide comprising a sequence having in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14. In a preferred embodiment, the TaWOX is a polypeptide that comprises the sequence represented by SEQ ID NO: 14. In another embodiment, the present invention provides the methods described above for increasing the efficiency of regenerating a genome edited plant cell into a plant and/or for decreasing the regeneration time, wherein TaWOX is a polypeptide having in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14.

The present invention thus provides a method for improving regeneration efficiency and/or speed of regeneration of one or more genome edited plant cell (s) into a plant, which method comprises the steps of
a) simultaneously introducing an EDTGM protein and a TaWOX protein into a plant cell,
b) allowing the EDTGM protein to modify the genome of said plant cell, and
c) regenerating said plant cell into a plant, thereby allowing the TaWOX protein to increase efficiency of regeneration and/or to shorten the regeneration time,
wherein said TaWOX protein has in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14 and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a mRNA encoding a TaWOX protein into a plant cell,
b) having the EDTGM mRNA and the TaWOX protein encoding mRNA transcribed in the cell,
c) allowing the transcribed EDTGM protein to modify the genome of said plant cell, and
d) regenerating said plant cell into a plant, thereby allowing the transcribed TaWOX protein to increase efficiency of regeneration and/or to shorten the regeneration time,
wherein said TaWOX protein has in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14 and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell(s) into a plant, which method comprises the steps of
a) simultaneously introducing an EDTGM protein and a mRNA encoding a TaWOX protein into a plant cell,
b) having the TaWOX protein encoding mRNA transcribed in the cell,
c) allowing the EDTGM protein to modify the genome of said plant cell, and
d) regenerating said plant cell into a plant, thereby allowing the transcribed TaWOX protein to increase efficiency of regeneration and/or to shorten the regeneration time,
wherein said TaWOX protein has in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14 and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX protein into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX protein to increase efficiency of regeneration and/or to shorten the regeneration time,
wherein said TaWOX protein has in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14 and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mixture of TaWOX protein and mRNA encoding a TaWOX protein into a plant cell,
b) having the mRNA encoding a TaWOX protein transcribed in the cell,
c) allowing the EDTGM protein to modify the genome of said plant cell, and
d) regenerating said plant cell into a plant, thereby allowing the TaWOX protein (transcribed and introduced) to increase efficiency of regeneration and/or to shorten the regeneration time,
wherein said TaWOX protein has in increasing order of preference at least 70%, 75%, 80%, 85%, 90%, 95%, 97% 97.2%, 97.5% 98%, 98.5%, 99%, 99.5%, 99.7%, 99.8%, 99.9% or more overall amino acid sequence identity to the TaWOX polypeptide represented by SEQ ID NO: 14 and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

More preferably, the TaWOX polypeptide is as represented by SEQ ID NO: 14.

Furthermore, the TaWOX polypeptide useful in the methods of the present invention also encompasses orthologues of the protein represented by SEQ ID NO: 14. Orthologues are a type of homologues that are encoded by genes from different organisms that have originated through speciation, and are also derived from a com-mon ancestral gene. Orthologues typically have the same or similar biological and functional properties as the gene from which they are derived (in casu promoting regeneration of a plant out of a plant cell), and can be identified by reciprocal BLAST. Reciprocal BLAST involves a first BLAST involving BLASTing a query sequence (for example SEQ ID NO: 14) against any sequence database, such as the publicly available NCBI database. BLASTP or TBLASTN (using standard default values) are generally used when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. An orthologue is identified if a high-ranking hit in the first BLAST is not from the same species as from which the query sequence is derived, and preferably results up-on BLAST back in the query sequence being among the highest hits. Web based tools for orthologue identification can be found in e.g. https://swissorthology.ch/service/search. Examples of TaWOX proteins that stimulate regeneration of genome edited cell are listed in Table A of Example 10. In one embodiment, it is beneficial to use a TaWOX that is substantially similar or identical to the endogenous TaWOX orthologue of SEQ ID NO: 14 that is naturally present in the genome edited plant cell. The term “endogenous” relates to any gene (and its encoded protein) or nucleic acid sequence that is naturally present in or native to the genome of a cell.

It is clear to the skilled person that the variants of SEQ ID NO: 14 as defined hereabove are most useful in the methods of the present invention if they have the same or similar biological and functional properties of SEQ ID NO: 14. Functional assays for characterizing a TaWOX are known in the art (such as assaying DNA binding activity, or modulating expression of auxin biosynthetic genes (Tian et al. Molecular Plant 7 (2): 277-289, 2014)), and a particular characteristic of SEQ ID NO: 14 is the capability of increasing the efficiency of the regeneration of plant cells into plants and/or of decreasing the regeneration time of plant cells into plants.

Also useful in the methods of the present invention, and provided that the same or similar biological and functional properties are retained as for the protein of SEQ ID NO: 14, are TaWOX proteins fused at their N-terminus or C-terminus with other proteins or tagging peptides including the binding domain or activation domain of a transcriptional activator as used in the yeast two-hybrid system, phage coat proteins, GUS, GFP (or other screenable markers), (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope (for a review of tagging peptides, see Terpe, Appl. Microbiol. Bio-technol. 60, 523-533, 2003). The present invention provides a method for improving regeneration efficiency and/or speed of regeneration of one or more genome edited plant cell(s) into a plant, which method comprises simultaneously introducing an EDTGM protein and a TaWOX protein (or their respective mRNAs) into a plant cell, wherein TaWOX is fused to a reporter protein, preferably a screenable marker, more preferably a fluorescent marker. A fusion protein that is particularly useful in the methods of this invention is a fusion of TaWOX with GFP (Green Fluorescent Protein), for example the fusion protein as represented by SEQ ID NO: 16 (wherein amino acids 1 to 217 represent the TaWOX part, amino acids 218 to 455 represent the GFP part, amino acids 456 to 463 represent a 3C protease site and amino acids 464 to 471 represent a His-tag with linker). It was observed that the TaWOX-GFP fusion protein had a better solubility than TaWOX alone while still retaining the capacity to increase the efficiency of regenerating genome edited plant cells and/or to decrease the regeneration time, and it is anticipated that variants of the TaWOX-GFP fusion protein retain the same functionality as that of the protein represented by SEQ ID NO: 16, which variants have at least 90%, 95%, 96%, 97%, 97.5% 98%, 98.5%, 99%, 99.5%, 99.7%, 99.8%, 99.9% or more amino acid sequence identity to the TaWOX fusion polypeptide represented by SEQ ID NO: 16. In another embodiment, the variants of the TaWOX-GFP fusion protein have in a local alignment at least 90%, 95%, 96%, 97%, 97.5% 98%, 98.5%, 99%, 99.5%, 99.7%, 99.8%, 99.9% or more amino acid sequence identity to the sequence spanning G211 to H471 in SEQ ID NO: 16. In a further embodiment, the variants of the TaWOX-GFP fusion protein have in a local alignment at least 90%, 95%, 96%, 97%, 97.5% 98%, 98.5%, 99%, 99.5%, 99.7%, 99.8%, 99.9% or more amino acid sequence identity to the sequence spanning M1 to V210 in SEQ ID NO: 16. As pointed out above, it is also anticipated that GFP in the TaWOX-GFP fusion protein can be replaced by other amino acid sequences for improving the solubility compared to TaWOX alone.

Therefore, the present invention furthermore provides a method for improving regeneration efficiency of a genome edited plant cell and/or for decreasing the regeneration time, which method comprises the steps of (a) simultaneously introducing an EDTGM protein and a TaWOX fusion protein, in the form of a protein or in the form of its encoding mRNA, into a plant cell, (b) allowing the EDTGM protein to modify the genome of said plant cell, and (c) regenerating said plant cell into a plant, thereby allowing the TaWOX fusion protein to increase efficiency of regeneration, or to shorten the regeneration time of said plant cell into a plant.

In a further embodiment, the present invention thus provides a method for improving regeneration efficiency and/or speed of regeneration of one or more genome edited plant cell (s) into a plant, which method comprises the steps of
a) simultaneously introducing an EDTGM protein and a TaWOX fusion protein into a plant cell,
b) allowing the EDTGM protein to modify the genome of said plant cell, and
c) regenerating said plant cell into a plant, thereby allowing the TaWOX fusion protein to increase efficiency of regeneration and/or to shorten the regeneration time,
wherein the regeneration efficiency is increased compared to the regeneration of corresponding genome-edited plant cells without having said TaWOX fusion introduced and/or wherein the regeneration time is decreased compared to the regeneration time of corresponding genome-edited plant cells without having said TaWOX fusion introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a mRNA encoding a TaWOX fusion protein into a plant cell,
b) having the EDTGM mRNA and the TaWOX fusion protein encoding mRNA transcribed in the cell,
c) allowing the transcribed EDTGM protein to modify the genome of said plant cell, and
d) regenerating said plant cell into a plant, thereby allowing the transcribed TaWOX fusion protein to increase efficiency of regeneration and/or to shorten the regeneration time;
wherein the regeneration efficiency is increased compared to the regeneration of corresponding genome-edited plant cells without having said TaWOX fusion introduced and/or wherein the regeneration time is decreased compared to the regeneration time of corresponding genome-edited plant cells without having said TaWOX fusion introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mRNA encoding a TaWOX fusion protein into a plant cell,
b) having the TaWOX fusion protein encoding mRNA transcribed in the cell,
c) allowing the EDTGM protein to modify the genome of said plant cell, and
d) regenerating said plant cell into a plant, thereby allowing the transcribed TaWOX fusion protein to increase efficiency of regeneration and/or to shorten the regeneration time;
wherein the regeneration efficiency is increased compared to the regeneration of corresponding genome-edited plant cells without having said TaWOX fusion introduced and/or wherein the regeneration time is decreased compared to the regeneration time of corresponding genome-edited plant cells without having said TaWOX fusion introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX fusion protein into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX fusion protein to increase efficiency of regeneration and/or to shorten the regeneration time;
wherein the regeneration efficiency is increased compared to the regeneration of corresponding genome-edited plant cells without having said TaWOX fusion introduced and/or wherein the regeneration time is decreased compared to the regeneration time of corresponding genome-edited plant cells without having said TaWOX fusion introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mixture of TaWOX fusion protein and mRNA encoding a TaWOX fusion protein into a plant cell,
b) having the mRNA encoding a TaWOX fusion protein transcribed in the cell,
c) allowing the EDTGM protein to modify the genome of said plant cell, and
d) regenerating said plant cell into a plant, thereby allowing the TaWOX fusion protein (transcribed and introduced) to increase efficiency of regeneration and/or to shorten the regeneration time;
wherein the regeneration efficiency is increased compared to the regeneration of corresponding genome-edited plant cells without having said TaWOX fusion introduced and/or wherein the regeneration time is decreased compared to the regeneration time of corresponding genome-edited plant cells without having said TaWOX fusion introduced.

Preferably, the TaWOX fusion protein has a better solubility than the TaWOX5 protein represented by SEQ ID NO: 14. More preferably, the TaWOX fusion protein is a sequence as represented by SEQ ID NO: 16.

In one aspect of the invention, the TaWOX fusion protein is a fusion between TaWOX as defined above with a screenable or selectable marker (a TaWOX/marker fusion protein).

“Selectable marker”, “screenable marker” or “reporter” includes any protein that confers a phenotype on a cell in which it is introduced expressed to facilitate the identification and/or selection of cells that are modified trough genome editing according to the methods of the present invention. These marker genes enable the identification of a successful transfer of the nucleic acid molecules or proteins via a series of different principles. Suitable markers may be selected from markers that confer antibi-otic or herbicide resistance, that introduce a new metabolic trait or that allow visual selection. Examples of selectable marker genes include genes conferring resistance to antibiotics, to herbicides, or genes that provide a metabolic trait. Screenable marker activity results in the formation of colour, luminescence or fluorescence. The skilled worker is familiar with such markers. Different markers are preferred, depending on the organism and the selection method. It is known that upon bombardment of plant cells with microparticles coated with proteins and/or nucleic acids, only a minority of the cells takes up the coated microparticles. To identify and select these cells, a screenable or selectable marker (such as the ones described above) can be introduced into the host cells together with the EDTGM and TaWOX (as protein of mRNA). Cells which carry the desired genome edit can be identified for example by screening for the presence or activity of the screenable or selectable marker.

The present invention thus also provides a method for screening genome edited plant cells having improved regeneration efficiency and/or speed of regeneration, which method comprises the steps of
a) simultaneously introducing an EDTGM protein, a TaWOX/marker fusion protein into a plant cell,
b) allowing the EDTGM protein to modify the genome of said plant cell,
c) screening and selecting a plant cell in which the screenable marker is present and/or active, and
d) regenerating the plant cell of step (c) into a plant, thereby allowing the TaWOX/marker fusion protein to increase efficiency of regeneration and/or to shorten the regeneration time;
wherein the regeneration efficiency is increased compared to the regeneration of corresponding genome-edited plant cells without having said TaWOX/marker introduced and/or wherein the regeneration time is decreased compared to the regeneration time of corresponding genome-edited plant cells without having said TaWOX/marker introduced.

In a further embodiment, the present invention provides a method for screening genome edited plant cells having improved regeneration efficiency and/or speed of regeneration, which method comprises the steps of
a) simultaneously introducing an EDTGM mRNA and a mRNA encoding a TaWOX/marker fusion protein into a plant cell,
b) having the EDTGM mRNA and the TaWOX/marker fusion protein encoding mRNA transcribed in the cell,
c) allowing the transcribed EDTGM protein to modify the genome of said plant cell, and
d) screening and selecting a plant cell in which the screenable marker is present and/or active, and
e) regenerating the plant cell of step (c) into a plant, thereby allowing the TaWOX/marker fusion protein to increase efficiency of regeneration and/or to shorten the regeneration time;
wherein the regeneration efficiency is increased compared to the regeneration of corresponding genome-edited plant cells without having said TaWOX/marker introduced and/or wherein the regeneration time is decreased compared to the regeneration time of corresponding genome-edited plant cells without having said TaWOX/marker introduced.

In a further embodiment, the present invention provides a method for screening genome edited plant cells having improved regeneration efficiency and/or speed of regeneration, which method comprises the steps of
a) simultaneously introducing an EDTGM protein and a mRNA encoding a TaWOX/marker fusion protein into a plant cell,
b) having the TaWOX/marker fusion protein encoding mRNA transcribed in the cell,
c) allowing the EDTGM protein to modify the genome of said plant cell, and d) screening and selecting a plant cell in which the screenable marker is present and/or active, and
e) regenerating the plant cell of step (c) into a plant, thereby allowing the TaWOX/marker fusion protein to increase efficiency of regeneration and/or to shorten the regeneration time;
wherein the regeneration efficiency is increased compared to the regeneration of corresponding genome-edited plant cells without having said TaWOX/marker introduced and/or wherein the regeneration time is decreased compared to the regeneration time of corresponding genome-edited plant cells without having said TaWOX/marker introduced.

In a further embodiment, the present invention provides a method for screening genome edited plant cells having improved regeneration efficiency and/or speed of regeneration, which method comprises the steps of
a) simultaneously introducing an EDTGM mRNA and a TaWOX/marker fusion protein into a plant cell,
b) having the EDTGM mRNA transcribed in the cell,
c) allowing the transcribed EDTGM protein to modify the genome of said plant cell, and
d) screening and selecting a plant cell in which the screenable marker is present and/or active, and
e) regenerating the plant cell of step (c) into a plant, thereby allowing the TaWOX/marker fusion protein to increase efficiency of regeneration and/or to shorten the regeneration time;
wherein the regeneration efficiency is increased compared to the regeneration of corresponding genome-edited plant cells without having said TaWOX/marker introduced and/or wherein the regeneration time is decreased compared to the regeneration time of corresponding genome-edited plant cells without having said TaWOX/marker introduced.

In a further embodiment, the present invention provides a method for screening genome edited plant cells having improved regeneration efficiency and/or speed of regeneration, which method comprises the steps of
a) simultaneously introducing an EDTGM protein and a mixture of TaWOX/marker fusion protein and mRNA encoding a TaWOX/marker fusion protein into a plant cell,
b) having the mRNA encoding a TaWOX/marker fusion protein transcribed in the cell,
c) allowing the EDTGM protein to modify the genome of said plant cell, and d) screening and selecting a plant cell in which the screenable marker is present and/or active, and
e) regenerating the plant cell of step (c) into a plant, thereby allowing the TaWOX/marker fusion protein to increase efficiency of regeneration and/or to shorten the regeneration time,
wherein the regeneration efficiency is increased compared to the regeneration of corresponding genome-edited plant cells without having said TaWOX/marker introduced and/or wherein the regeneration time is decreased compared to the regeneration time of corresponding genome-edited plant cells without having said TaWOX/marker introduced.

In an alternative embodiment of the methods described herein, the TaWOX and the marker are introduced into a plant cell as separate proteins or mRNAs, next to the EDTGM protein.

Also useful in the methods of the present invention are functional fragments of TaWox proteins. The term “functional fragment” refers to any protein which represents merely a part of the full length protein, but still has substantially the same biological activity of the full length protein. TaWOX proteins are transcription factors and (at least in their native form) typically have DNA binding activity. It is known that the Homeodomain is responsible for DNA binding, and tools and techniques for measuring DNA binding activity are well known in the art and include for example electrophoretic mobility shift assays (EMSA) or DNase footprinting. In addition, functional characterization of the EAR motif and the WUS-box motif have shown that these are involved in repression, and that the EAR motif interacts with TOPLESS (TPL) and TPL-related proteins (Bûrglin & Affolter, Chromosoma 125:497-521, 2016). Therefore, these domains are important for the functionality of the TaWOX protein.

In a further embodiment, the present invention thus provides a method for improving regeneration efficiency and/or speed of regeneration of one or more genome edited plant cell (s) into a plant, which method comprises the steps of
a) simultaneously introducing an EDTGM protein and a functional fragment of a TaWOX protein into a plant cell,
b) allowing the EDTGM protein to modify the genome of said plant cell, and
c) regenerating said plant cell into a plant, thereby allowing the TaWOX functional fragment to increase efficiency of regeneration and/or to shorten the regeneration time;
wherein the regeneration efficiency is increased compared to the regeneration of corresponding genome-edited plant cells without having said TaWOX functional fragment introduced and/or wherein the regeneration time is decreased compared to the regeneration time of corresponding genome-edited plant cells without having said TaWOX functional fragment introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a mRNA encoding a functional fragment of a TaWOX protein into a plant cell,
b) having the EDTGM mRNA and the TaWOX functional fragment encoding mRNA transcribed in the cell,
c) allowing the transcribed EDTGM protein to modify the genome of said plant cell, and
d) regenerating said plant cell into a plant, thereby allowing the transcribed TaWOX functional fragment to increase efficiency of regeneration and/or to shorten the regeneration time;
wherein the regeneration efficiency is increased compared to the regeneration of corresponding genome-edited plant cells without having said TaWOX functional fragment introduced and/or wherein the regeneration time is decreased compared to the regeneration time of corresponding genome-edited plant cells without having said TaWOX functional fragment introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mRNA encoding a functional fragment of a TaWOX protein into a plant cell,
b) having the TaWOX functional fragment encoding mRNA transcribed in the cell,
c) allowing the EDTGM protein to modify the genome of said plant cell, and
d) regenerating said plant cell into a plant, thereby allowing the transcribed TaWOX functional fragment to increase efficiency of regeneration and/or to shorten the regeneration time;
wherein the regeneration efficiency is increased compared to the regeneration of corresponding genome-edited plant cells without having said TaWOX functional fragment introduced and/or wherein the regeneration time is decreased compared to the regeneration time of corresponding genome-edited plant cells without having said TaWOX functional fragment introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a functional fragment of a TaWOX protein into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX functional fragment to increase efficiency of regeneration and/or to shorten the regeneration time;
wherein the regeneration efficiency is increased compared to the regeneration of corresponding genome-edited plant cells without having said TaWOX functional fragment introduced and/or wherein the regeneration time is decreased compared to the regeneration time of corresponding genome-edited plant cells without having said TaWOX functional fragment introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mixture of a functional fragment of a TaWOX protein and mRNA encoding a TaWOX functional fragment into a plant cell,
b) having the mRNA encoding the TaWOX functional fragment transcribed in the cell,
c) allowing the EDTGM protein to modify the genome of said plant cell, and
d) regenerating said plant cell into a plant, thereby allowing the TaWOX functional fragment (transcribed and introduced) to increase efficiency of regeneration and/or to shorten the regeneration time,
wherein the regeneration efficiency is increased compared to the regeneration of corresponding genome-edited plant cells without having said TaWOX functional fragment introduced and/or wherein the regeneration time is decreased compared to the regeneration time of corresponding genome-edited plant cells without having said TaWOX functional fragment introduced.

In a preferred embodiment of the present invention, the TaWOX polypeptide useful in the methods of the present invention is represented by SEQ ID NO: 14, or has at least 97.2%, preferably at least 97.5%, more preferably at least 98%, furthermore preferably at least 98.5%, furthermore preferably at least 99% sequence identity to SEQ ID NO: 14.

The sequence identity is determined using a global alignment algorithm, such as the Needleman Wunsch algorithm in Geneious Prime (Biomatters Ltd.), preferably with default parameters and preferably with sequences of mature proteins (i.e. without taking into account secretion signals or transit peptides). In one embodiment the sequence identity level is determined by comparison of the polypeptide sequences over the entire length of the sequence of SEQ ID NO: 14. In another embodiment the sequence identity level is determined by comparison of the polypeptide sequences over the entire length of the sequence of SEQ ID NO: 29.

Furthermore, the skilled person will readily appreciate that the methods of the present invention are not limited to the use of TaWOX proteins, but that any protein promoting the regeneration of a genome edited plant cell into a plant can be used as well, as long as that particular regeneration promoting protein is co-introduced with the nuclease that will perform the desired genome modification. Examples of such proteins promoting the regeneration of a genome edited plant cell into a plant include WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants, provided that these functional fragments or variants retain the same or similar biological and functional properties as the protein from which they are derived.

Therefore, the present invention also provides a method for increasing the efficiency of regenerating a genome edited plant cell into a plant and/or of decreasing the regeneration time, comprising the steps of:
(a) simultaneously introducing into a plant cell (i) an EDTGM protein and (ii) a protein promoting plant regeneration, wherein said endonuclease and said protein promoting plant regeneration are introduced as protein or as its encoding mRNA;
(b) allowing the endonuclease to modify the genome of said plant cell;
(c) regenerating said plant cell into a plant, thereby allowing the introduced protein promoting plant regeneration to increase efficiency of regeneration and/or to decrease the regeneration time; wherein the regeneration efficiency is increased compared to the regeneration of corresponding genome-edited plant cells without having said protein promoting plant regeneration introduced and wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants and/or wherein the regeneration time is decreased compared to the regeneration time of corresponding genome-edited plant cells without having said protein promoting plant regeneration introduced and wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants.

In a further embodiment, the present invention provides a method for improving regeneration efficiency and/or speed of regeneration of one or more genome edited plant cell (s) into a plant, which method comprises the steps of
a) simultaneously introducing an EDTGM protein and a protein promoting plant regeneration into a plant cell,
b) allowing the EDTGM protein to modify the genome of said plant cell, and
c) regenerating said plant cell into a plant, thereby allowing the protein promoting plant regeneration to increase efficiency of regeneration and/or to shorten the regeneration time,
wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a mRNA encoding a protein promoting plant regeneration into a plant cell,
b) having the EDTGM mRNA and the protein promoting plant regeneration mRNA transcribed in the cell,
c) allowing the transcribed EDTGM protein to modify the genome of said plant cell, and
d) regenerating said plant cell into a plant, thereby allowing the transcribed protein promoting plant regeneration to increase efficiency of regeneration and/or to shorten the regeneration time,
wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mRNA encoding a protein promoting plant regeneration into a plant cell,
b) having the protein promoting plant regeneration mRNA transcribed in the cell,
c) allowing the EDTGM protein to modify the genome of said plant cell, and
d) regenerating said plant cell into a plant, thereby allowing the transcribed protein promoting plant regeneration to increase efficiency of regeneration and/or to shorten the regeneration time,
wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a protein promoting plant regeneration into a plant cell,
b) having the EDTGM mRNA transcribed in the cell,
c) allowing the transcribed EDTGM protein to modify the genome of said plant cell, and
d) regenerating said plant cell into a plant, thereby allowing the protein promoting plant regeneration to increase efficiency of regeneration and/or to shorten the regeneration time;
wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mixture of protein promoting plant regeneration and mRNA encoding protein promoting plant regeneration into a plant cell,
b) having the protein promoting plant regeneration mRNA transcribed in the cell,
c) allowing the EDTGM protein to modify the genome of said plant cell, and
d) regenerating said plant cell into a plant, thereby allowing the protein promoting plant regeneration (transcribed or introduced) to increase efficiency of regeneration and/or to shorten the regeneration time;
wherein said protein promoting plant regeneration is selected from WUSCHEL (WUS), Growth Regulating Factor (GRF), Homeodomain Leucine Zipper (HDZipII), BABY BOOM (BBM), NO TRANSMITTING TRACT (NTT), KNOTTED1, Ovule Development Protein 2 (ODP2), isopentenyltransferase, clavata3 mutant and their functional fragments or variants and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

The present invention also provides a method for increasing the efficiency of regenerating a genome edited plant cell into a plant and/or for decreasing the time required for regeneration into a plant, as described above, wherein next to the TaWOX also another protein promoting plant regeneration (or its encoding mRNA) is introduced into a plant cell.

The term “regeneration” as used herein means generation of a plant by in vitro culturing of plant cells, protoplasts or callus tissue and can be achieved via somatic embryogenesis or organogenesis. Methods for regenerating plants are well known in the art (Haberlandt, G. (1902), Sitzungsber Akad. Wiss. Wien. Math. Nat. 111, 69-91; Skoog and Miller (1957), Symp. Soc. Exp. Biol. 54, 118-130; Steward et al. (1958), Am. J. Bot. 45, 705-708, doi.org/10.2307/2439728). Many of these methods encompass cultivation on media with particular ratios of hormone concentrations, with or without selection with a screenable or selectable marker. Genome-edited cells to be regenerated may be derived from various tissues or organs (such as roots, shoots, leaves, petioles, gametophytes, embryo's (mature or immature), meristems), with or without a step of callus formation.

It is clear for the skilled person that the increased regeneration efficiency or decreased time required for regeneration is compared to the regeneration efficiency or regeneration time of corresponding control plants, that is, corresponding genome-edited plant cells without having said TaWOX protein, functional fragment, protein fusion, orthologue or homologue thereof, or other protein promoting plant regeneration introduced, but that have otherwise received the same treatment.

The present invention provides a method for improving regeneration efficiency and/or regeneration time of a genome edited plant cell, comprising the simultaneously introduction of an EDTGM protein and a TaWOX, in the form of a protein or its encoding mRNA, into a plant cell, further comprising the modification of the genome of said plant cell by the endonuclease, and the regeneration said plant cell into a plant, thereby allowing the TaWOX protein to increase efficiency of regeneration. In a particular embodiment, the increased regeneration efficiency is an increased percentage of modified plant cells that regenerate into mature plants, compared to the percentage of control plants regenerated from genome edited plant cells that had undergone the same procedure except that they were not treated with TaWOX.

The term “mRNA” or “messenger RNA” refers to a single stranded RNA template for protein synthesis, comprising a 5′ cap and a polyadenylated 3′ end and encoding one or more proteins. Exogenously introduced mRNA is recognized in the cell by the protein-synthesizing machinery and is eventually translated to its encoded protein (s).

The term “genome” refers to the whole of the genetic content of a cell, including its mitochondrial or chloroplast DNA and “genome modification” or “genome edit” or “gene edit” as used herein means a change in the sequence of the genome, such as insertions, deletions, substitutions, translocations and the like, deliberately introduced by human intervention through the use of endonucleases or variants thereof, as described hereunder.

The methods used in the present invention comprise a step of simultaneous introduction of a EDTGM protein and a TaWOX (as protein or as mRNA). Endonucleases for introducing a desired genome modification are well known in the art and encompass technologies like zinc finger nucleases (ZFNs), transcription activator-like effector nucleases (TALENs), meganucleases and CRISPR-Cas systems. Out of scope are the classical restriction enzymes (Type II restriction enzymes) having short palindromic sequence recognition sites (usually between 4 and 8 bases).

CRISPR-Cas systems are currently the most widely used technologies for precise genome editing. Cas (CRISPR associated) proteins are non-specific endonucleases which associate with a guide RNA (gRNA) that can be designed to direct the Cas protein to a specific sequence on the genome for performing a DNA cleavage at a specific site. As used herein, an “endonuclease designed for a desired genome modification” or “EDTGM” protein, in case it is a CRISPR-Cas system, refers to the Cas protein complexed with the gRNA designed for targeting the Cas protein to a specific genomic site for creating a desired genome edit.

Among the various known Cas (CRISPR associated) proteins, Cas9 and Cas12a (Cpf1) have the broadest range of applications in genome editing. Whereas Cas9 normally complexes to a transactivating CRISPR RNA (tracrRNA) and a CRISPR RNA (crRNA) which contains the spacer sequence leading the Cas protein to the target site in the genome, nowadays mostly synthetic or single guide RNAs (sgRNA) are used that combine the function of the tracrRNA and the crRNA to create a fully functional CRISPR-Cas system when associated with a Cas9 protein. Cas12a on the other hand only requires a single crRNA. In essence CRISPR-Cas systems can thus be regarded as a two-component system. The term gRNA as used herein not only encompasses combinations of tracrRNA and designed crRNA, but also single gRNA, or any other RNA capable of directing the Cas protein to the desired target site in the genome. Furthermore, it is to be understood that where an “EDTGM”, in case it is a CRISPR-Cas system, is introduced in a cell in the form of mRNA, the mRNA encoding the Cas protein is accompanied by a suitable gRNA such that when the mRNA is translated into a Cas protein, the Cas protein will form a complex with the accompanying gRNA to form a functional CRISPR-Cas system in that cell.

Whereas Cas proteins typically generate dsDNA breaks that can result in specific genome edits, modifications of the Cas proteins have broadened their range of activities, including, but not limited to, nickase activity, base editing, prime editing, RNA editing, transcriptional activation or repression, or epigenome editing. In some cases, the Cas proteins were mutated to alter their activities, in other cases fusions with peptides or polypeptides were created to obtain new functionalities, or combinations thereof. For example, Halperin et al. (Nature 560 (7717): 248-252, 2018) used a Cas9 mutant with nickase activity, fused to a DNA polymerase to perform targeted gene editing. Because all these Cas variants are derived from a wild type Cas endonuclease, the term “EDTGM” as used in the present invention encompasses such Cas variants as well.

TAL (transcription activator-like) effectors (TALEs) are proteins secreted by Xanthomonas bacteria via their type III secretion system when they infect various plant species. They recognize plant DNA sequences through a central repeat domain consisting of a variable number of 34 amino acid repeats. Within this central repeat domain two polymorphic amino acid residues at positions 12 and 13 form the repeat-variable di-residue (RVD) in which the amino acid at position 13 is responsible for the preferential recognition of the repeat module to a single specific nucleotide. There is a one-to-one correspondence between the identity of these two critical amino acids in each repeat and each DNA base in the target sequence. The simple correspondence between amino acids in TALE and DNA bases in their target sites makes them useful for genome engineering applications, as it is possible to program the TALE DNA binding domain to recognize specific DNA sequences. A TAL effector nuclease (TALEN) is a fusion protein composed of a TALE DNA binding domain and a restriction enzyme, such as Fokl endonuclease. Because Fokl functions as a dimer to cleave DNA, 2 modules of a TALE DNA binding domain and a Fokl nuclease domain are required, with the TALE DNA binding domains carefully designed to target non-palindromic DNA sites where applicable and to obtain correct spacing and orientation of both modules on the target site in the genome DNA cleavage. TALE DNA binding domains can also be fused to other nucleases, such as Pvull, or to meganucleases (truncated I-Tevl, I-Anil or I-Onul). Similarly to CRISPR-Cas systems, TALE DNA binding domains can furthermore be used for constructing TALE base editors, TALE transposases, TALE recombinases (reviewed in Becker and Boch, Gene and Genome Editing 2 (2021) 100007). All these variants are useful in the methods of the present invention.

Just like TALENs, Zinc-finger nucleases are a synthetic fusion between a restriction enzyme and a Zinc finger (Znf) DNA binding domain. These domains are relatively small protein motifs that contain multiple finger-like protrusions that make tandem contacts with their target molecule. Some of these domains bind zinc, or other metals, others can bind nucleic acids or proteins. Their binding properties depend on the amino acid sequence of the finger domains and on the linker between fingers, as well as on the higher-order structures and the number of fingers. ZnF motifs occur in several unrelated protein super families, varying in both sequence and structure, and they display considerable versatility in binding modes. Since the molecular structure of zinc fingers domains are well known and their nucleotide base recognition understood, it is therefore possible to generate ‘programmable’ zinc finger domains which can recognize specific DNA sequences. Each individual Zn-finger can recognize a stretch of 3 DNA base pairs, and by combining 2 or 3 Zn-finger domains per module, that each interact with a defined stretch of 3 bp, a specific DNA targeting site of 12 to 18 base pairs can be designed. The specificity of the Zn-finger elements will define the level of off-target cleavage. Zn-finger nuclease typically use the Fokl endonuclease, in which case the Zn-finger nuclease is composed of 2 modules, each being a fusion between a zinc-finger DNA binding domain and the Fokl nuclease domain. Zn finger domains have also been fused to deaminases to generate base editors (Lim et al., Nature Communications, doi.org/10.1038/s41467-022-27962-0). Both TALEN and Zn-finger nucleases have nickase versions as well.

The fourth major group of EDTGM comprise the meganucleases or homing endonucleases. These are nucleases with a long recognition sequence (typically between 12 to 40 base pairs), resulting in a high target sequence specificity. However, to become practically useful their DNA-recognition specificity must be modifiable, which has proven to be difficult. Extensive structure-function relationship studies, computer modelling and mutagenesis allowed to create endonuclease libraries with altered DNA binding specificities. The group of homing endonucleases consists of several families, of which the LAGLIDADG family is most known. Members of this family have 1 or 2 LAGLIDADG motifs (SEQ ID NO: 39) that are implicated in DNA cutting. Well known examples are PI-SceI and I-CreI nucleases. Yet, the cost and timewise burden of generating homing endonuclease variants is too high for becoming a valuable alternative to Zn-finger or TALE based endonucleases that have much more combinatorial possibilities.

In a preferred embodiment however, the EDTGM, used for targeted genome modification, is a CRISPR-Cas system. The term CRISPR-Cas system as used herein refers to the combination of a Cas protein (having nuclease or nickase activity, or being a dead Cas) and its corresponding crRNA and tracrRNA (such as for Cas9), or the crRNA as for Cas12a, or guide RNA (gRNA or sgRNA). Preferably the combination results in a Cas protein, capable of targeting a desired site in the genome and optionally capable of creating a double strand break, or having nickase activity. Preferably the CRISPR-Cas system is a Class 2 CRISPR-Cas system, more preferably a type II or a type V CRISPR-Cas system (Wright et al., Cell. 164 (1-2): 29-44, 2016). In one embodiment the Cas protein is Cas12a (Cpf1) isolated from Acidaminococcus sp. BV3L6 or from Lachnospiraceae_bacterium_MC2017, or a variant thereof. Also encompassed are modified Cas proteins having altered PAM recognition sites (such as the RR, RV and RVR mutants, or the E795L variant of AsCas12 (Gao et al. Nat Biotechnol. 2017; 35(8): 789-792. doi:10.1038/nbt.3900, WO 2018/035388, WO 2020/172502). Moreover, a person skilled in the art is aware of alternative CRISPR-Cas systems (Makarova and Koonin, Methods Mol Biol. 2015; 1311: 47-75. doi:10.1007/978-1-4939-2687-9_4) that can be used in the methods of the present invention.

As pointed out above, the targeted genome modification and subsequent regeneration of the plant cell with the modified genome into a plant can be performed by simultaneously introducing a suitable EDTGM and a TaWOX, either in the form of a protein or in the form of a mRNA encoding such protein.

All the above-mentioned tools for genome editing (be it TALENs, Zn-finger proteins, meganucleases or CRISPR-Cas systems in its variant forms) not necessarily need to be introduced in a cell by expressing an exogenously introduced coding DNA sequence, i.e. by transformation. In a preferred embodiment, they are delivered in the form of a protein or its encoding mRNA. Advantageously they are co-introduced into the cell together with a TaWOX protein, or its encoding mRNA. In case of a CRISPR-Cas complex, this complex may be prepared at forehand before introduction into the cell by incubating the Cas protein with the desired sgRNA, a crRNA or a crRNA/tracrRNA mixture in a suitable ratio, such that an active ribonucleoprotein is formed. The Cas protein can also be incubated with two or more different gRNAs, such that a mixture of ribonucleoproteins is formed, targeting different sites in the genome. Methods for preparing ribonucleoproteins are known in the art. In a preferred embodiment, an active ribonucleoprotein is introduced alongside the TaWOX. As mentioned above, in case a Cas encoding mRNA is used, the required sgRNA, crRNA or crRNA/tracrRNA mixture (or a mRNA mimic thereof) are introduced together with the Cas mRNA. Therefore, in one aspect of the invention, both the TaWOX and the EDTGM are introduced in the form of a protein. In another aspect of the invention, the TaWOX is introduced in the form of a mRNA and the EDTGM in the form of a protein. In still another aspect the TaWOX is introduced in the form of protein and the EDTGM is introduced as a mRNA (where in case the endonuclease is a CRISPR-Cas system, the Cas mRNA is introduced together with its gRNA or its crRNA and tracrRNA). In yet another aspect of the invention, both the TaWOX and the EDTGM are introduced in the form of a mRNA. In a particular embodiment the TaWOX and the EDTGM are not introduced as separate proteins or separate mRNAs, but as a fusion protein, which is then encoded by a single mRNA. It should be noted that also other compounds can be co-introduced next to the TaWOX and the EDTGM, for example a donor or repair template where a CRISPR-Cas system is introduced for promoting homology directed repair.

Methods of introducing the TaWOX and EDTGM, either as protein or as mRNA, can be by chemical means, non-chemical means or by physical means. Chemical means for introducing proteins or nucleic acids in a cell usually rely on uptake by endocytosis or on merger with the cell membrane. Non limiting examples of chemical means for introduction include lipofection, polyethyleneimine (PEI)-mediated introduction, polyethylene glycol (PEG)-mediated introduction, nucleofection, calcium phosphate precipitation, liposomes, immunoliposomes, fusion, polycation or lipid:nucleic acid conjugates, cell-penetrating peptides, and DEAE-dextran mediated transfection.

Non-chemical means encompass methods that create pores in the cell membrane or spots of increased membrane permeability. Well known examples are electroporation, sonoporation (by cavitation of gas bubbles), and use of laser light. Non limiting examples of physical means for introduction include microinjection, nanoparticle-mediated delivery, particle gun technology (biolistics) and impalefection (trough use of needle-like nanostructures coated with the compound(s) of interest.

In a preferred embodiment of the invention, the TaWOX and EDTGM are introduced by physical means, preferably by biolistics. Biolistics or particle bombardment is a tool for delivering compound(s) into a cell by coating the compound(s) on metal microparticles (usually tungsten or gold particles) and shooting these coated particles with a gene gun into the cell. This technique has been proven to be very useful for transforming plants that are otherwise hard to transform or regenerate, as well as for transformation of cell organelles such as protoplasts (for a review, see Ozyigit and Kurtoglu, Mol Biol Rep. 47(12):9831-9847, 2020). Another useful method is aerosol beam microinjection (U.S. Pat. No. 5,240,842).

In one embodiment, the EDTGM protein or mRNA and the TaWOX protein or mRNA are coated on the same (batch of) microparticle(s); as a result the EDTGM protein or mRNA and the TaWOX protein or mRNA are co-introduced into the same cell. In another embodiment, the EDTGM protein or mRNA is coated on one batch of microparticles and the TaWOX protein or mRNA on a second batch of microparticles. Next, both batches of microparticles are combined before bombarding the cell. This allows to generate batches of microparticles with varying ratios of EDTGM protein or mRNA and TaWOX protein or mRNA.

As a result, after bombardment not all cells comprising an EDTGM protein or mRNA will also comprise the TaWOX protein or mRNA, but these cells may benefit from the presence and biological activity of the TaWOX protein or mRNA in neighbouring cells.

In a further embodiment, the present invention provides a method for improving regeneration efficiency and/or speed of regeneration of one or more genome edited plant cell (s) into a plant, which method comprises the steps of
a) simultaneously introducing an EDTGM protein and a TaWOX protein by biolistics into a plant cell,
b) allowing the EDTGM protein to modify the genome of said plant cell, and
c) regenerating said plant cell into a plant, thereby allowing the TaWOX protein to increase efficiency of regeneration and/or to shorten the regeneration time;
wherein the EDTGM protein and the TaWOX protein are coated on a single batch of microparticles or wherein the EDTGM protein and the TaWOX protein are coated on separate batches of microparticles that are subsequently combined prior to the biolistics step and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX mRNA by biolistics into a plant cell,
b) having the EDTGM mRNA and the TaWOX mRNA transcribed in the cell,
c) allowing the transcribed EDTGM protein to modify the genome of said plant cell, and
d) regenerating said plant cell into a plant, thereby allowing the transcribed TaWOX protein to increase efficiency of regeneration and/or to shorten the regeneration time;
wherein the EDTGM protein and the TaWOX protein are coated on a single batch of microparticles or wherein the EDTGM protein and the TaWOX protein are coated on separate batches of microparticles that are subsequently combined prior to the biolistics step and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a TaWOX mRNA by biolistics into a plant cell,
b) having the TaWOX mRNA transcribed in the cell,
c) allowing the EDTGM protein to modify the genome of said plant cell, and
d) regenerating said plant cell into a plant, thereby allowing the transcribed TaWOX protein to increase efficiency of regeneration and/or to shorten the regeneration time;
wherein the EDTGM protein and the TaWOX protein are coated on a single batch of microparticles or wherein the EDTGM protein and the TaWOX protein are coated on separate batches of microparticles that are subsequently combined prior to the biolistics step; wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM mRNA and a TaWOX protein by biolistics into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX protein to increase efficiency of regeneration and/or to shorten the regeneration time;
wherein the EDTGM protein and the TaWOX protein are coated on a single batch of microparticles or wherein the EDTGM protein and the TaWOX protein are coated on separate batches of microparticles that are subsequently combined prior to the biolistics step and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for improving regeneration efficiency and/or speed of regeneration of one or more genome edited plant cell (s) into a plant, which method comprises the steps of
a) simultaneously introducing an EDTGM protein and a mixture of TaWOX protein and TaWOX mRNA by biolistics into a plant cell,
b) having the TaWOX mRNA transcribed in the cell,
c) allowing the EDTGM protein to modify the genome of said plant cell, and
d) regenerating said plant cell into a plant, thereby allowing the TaWOX protein (transcribed or introduced) to increase efficiency of regeneration and/or to shorten the regeneration time,
wherein the EDTGM protein and the TaWOX protein/mRNA mixture are coated on a single batch of microparticles or wherein the EDTGM protein and the TaWOX protein/mRNA mixture are coated on separate batches of microparticles that are subsequently combined prior to the biolistics step and wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

The present invention also provides a microparticle coated with a EDTGM protein and a TaWOX protein. The present invention furthermore provides a microparticle coated with a mRNA encoding an EDTGM protein and a mRNA encoding a TaWOX protein.

The present also provides a plant cell comprising a microparticle coated with an EDTGM protein and a TaWOX protein, or a microparticle coated with a mRNA encoding an EDTGM and a mRNA encoding a TaWOX protein. The present invention also provides a plant cell comprising a microparticle coated with an EDTGM protein or its encoding mRNA, and a microparticle coated with a TaWOX protein and/or its encoding mRNA.

The present invention also provides a microparticle coated with a EDTGM, a TaWOX and a screenable or selectable marker, either as protein or as mRNA or in combinations thereof.

The present invention furthermore provides a gene editing system, comprising one of:
a microparticle coated with a EDTGM protein and a TaWOX protein.
a microparticle coated with a mRNA encoding an EDTGM protein and a mRNA encoding a TaWOX protein.
a microparticle coated with an EDTGM protein and a TaWOX protein, or a microparticle coated with a mRNA encoding an EDTGM and a mRNA encoding a TaWOX protein.
a microparticle coated with an EDTGM protein or its encoding mRNA, and a microparticle coated with a TaWOX protein and/or its encoding mRNA.
a microparticle coated with a EDTGM, a TaWOX and a screenable or selectable marker, either as protein or as mRNA or in combinations thereof.

The present also provides a plant cell comprising a microparticle coated with an EDTGM, a TaWOX and a screenable or selectable marker as proteins, as mRNA or in combinations thereof. In one aspect, the TaWOX and the screenable or selectable marker are present as separate proteins or mRNAs, in another aspect the TaWOX and the screenable or selectable marker are present as a fusion protein or encoded on a single mRNA.

Following the simultaneous introduction of the EDTGM protein and the TaWOX (either as a protein or as a mRNA), there is a step of genome modification by the endonuclease. The endonucleases designed for a desired genome modification described above, and in particular the CRISPR-Cas systems are versatile tools for generating a wide variety of genome modifications:

Creation of a double strand break (ds break), regardless of the endonuclease used will trigger a repair mechanism in the cell. The DNA double-strand breaks caused by the endonucleases designed for a desired genome modification can be re-paired by 2 mechanisms. The Non-Homologous End Joining (NHEJ) mechanism directly anneals the loose ends and is error-prone, resulting in insertions, deletions, substitutions or recombinations at the cleavage site after repair. When occurring in a gene sequence, NHEJ repair could result in a gene knock out or in a mutated sequence coding for a protein having an altered function, or in promoter modification/inactivation. Homology Directed Repair is much more precise because it is based on a homologous DNA sequence in the nucleus, or based on the sequence of an introduced exogenous repair template. By designing the repair template it is possible to introduce specific mutations (deletions, insertions, substitutions) at or around the cleavage site in the genome, provided that the repair template is at its 3′ and 5′ end homologous to the sequences flanking the double-strand break in the genome. The insertions can range from a single nucleotide to a sequence spanning multiple nucleotides. The donor DNA can be a single strand oligonucleotide for smaller insertions, but for larger insertions (i.e. more than 200 nucleotides), a template comprised in a double strand plasmid is better suited. Practical applications are for example gene replacement or gene knock-in. CRISPR-Cas systems, and in particular Cas9 or Cas12a (Cpf1) are nowadays the most popular tools for creating particular genome edits. Whereas Cas9 has two DNA cutting domains, each cleaving one of the strands, Cas12a has a single DNA cleaving domain that cuts both DNA strands. In addition mutant Cas9 proteins were designed in which one of the DNA cutting domains were inactivated (for example, the D10A mutation inactivates the RuvC domain, the H840A mutation inactivates the HNH domain in Cas9), resulting in the formation of a nickase that creates staggered cuts. Cas nickases have the advantage of improving repair levels beyond 10 bp away from the cut site, thereby reducing the frequency of off-target edits.

An important enhancement of CRISPR-Cas functionality was the development of base editors that create specific point mutations with a reduced amount of unwanted side products. Cytosine base editors introduce C to T changes, whereas adenosine base editors introduce A to G changes in the genome. To this end, a deaminase is fused to a Cas nuclease or a Cas nickase or an inactivated Cas protein, whereby the Cas protein is directed to the a specific site on the genome and positions itself so that the target base can be edited by the deaminase. Since their first creation, the base editors were further optimised so that the editing efficiency increased, the off-target effects were reduced and the targeting scope widened.

Prime editors represent a further improvement of the CRISPR-Cas technology, and allow to introduce targeted indels, and all types of base-to-base conversions. This technique requires a fusion of a Cas9 nickase with a reverse transcriptase to create a prime editor, and a specially designed prime editing guide RNA (pegRNA). The pegRNA combines the gRNA and the reverse transcriptase template with the intended edits flanked by sequences complementary to the genomic DNA strand that will be nicked. In a first step, the Cas9 nickase is guided to the target site and cleaves the target strand, thereafter the reverse transcriptase domain uses the pegRNA to reverse transcribe of the desired edit onto the nicked target DNA strand, thereby creating a heteroduplex which upon copying will introduce the edit in unedited strand.

Also RNA can be edited with CRISPR-Cas systems, in particular with Cas13a. This Cas protein shares no homology with other Cas proteins and has a binding preference for RNA rather than for DNA, hence it allows for mRNA or other RNA editing without altering the genome. It finds application in diagnostics but also as an editing tool in eukaryotic cells, engineered Cas13 proteins were developed for RNA base editing.

Inactivated Cas proteins (dead Cas or dCas) can be fused to transcriptional activators or repressors by fusing the dCas protein with a transcriptional activator or repressor and targeting the fusion protein with a suitable gRNA to a site upstream of, or on the promoter of a gene. Likewise, dCAS proteins can be fused to epigenetic modifiers like demethylases or acyltransferases for modifying the epigenome.

Any of the above described genome editing tools can be used in such embodiment of the present invention.

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

Plant cells upon which the methods of the present invention can be applied include but are not limited to cells from suspension cultures, callus tissue, embryos, meristematic regions, gametophytes, sporophytes, pollen and microspores. The plant cells can be derived from seeds, plant tissues or organs, including but not limited to shoots, stems, leaves, roots (including tubers), flowers. Plant cells can be differentiated or undifferentiated (e.g. callus, suspension culture cells, protoplasts, leaf cells, root cells, phloem cells, pollen).

Genome edited plant cells can be regenerated via all methods with which the skilled worker is familiar. Regeneration is the process wherein a plant cell divides on a particular growth medium in which the amount and ratio of phytohormones is carefully controlled. The dividing cells ultimately form tissue, organs or complete plants. Pollen and microspores can be used for generating haploid plants. Suitable methods can be found in for example Evans et al., “Protoplasts Isolation and Culture, Handbook of Plant Cell Culture”, p. 124-176, MacMillilan Publishing Company, New York, 1983, in “Transgenic Plants, Vol. 1, Engineering and Utilization”, eds. S. D. Kung and R. Wu, Academic Press (1993) p.128-143, in Potrykus (Annu. Rev. Plant Physiol. Plant Molec. Biol. 42 (1991) 205-225)), or in “Binding, Regeneration of Plants, Plant Protoplasts”, p. 21-73, CRC Press, Boca Raton, 1985.

The increased efficiency of regeneration of the genome edited plant cells can manifest itself as for example a shorter time to obtain complete plants, or as higher percentage of fully regenerated plants, compared to the regeneration of control plant cells. Control plant cells as used herein are plant cells of the same origin and that have undergone the same treatment, except that no TaWOX was introduced. In a preferred embodiment, the increased efficiency of regeneration is manifested as a higher percentage of genome edited cells that develop into complete plants and/or as a shorter time required for genome edited cells to develop into complete plants. Therefore the methods of the present invention are well suited for genome editing of plants of which the cells are recalcitrant to regeneration.

The present invention also provides a method for producing a genome edited plant comprising the steps of
(a) simultaneously introducing into a plant cell (i) an EDTGM and (ii) a TaWOX, wherein said EDTGM and said TaWOX are introduced as protein or as its encoding mRNA;
(b) allowing the endonuclease to modify the genome of said plant cell;
(c) regenerating said plant cell into a plant, thereby allowing the introduced TaWOX to increase efficiency of regeneration;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method producing a genome edited plant, which method comprises the steps of
a) simultaneously introducing an EDTGM protein and a TaWOX protein into a plant cell,
b) allowing the EDTGM protein to modify the genome of said plant cell, and
c) regenerating said plant cell into a plant, thereby allowing the TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for producing a genome edited plant, which method comprises the steps of
a) simultaneously introducing an EDTGM mRNA and a TaWOX mRNA into a plant cell,
b) having the EDTGM mRNA and the TaWOX mRNA transcribed in the cell,
c) allowing the transcribed EDTGM protein to modify the genome of said plant cell, and
d) regenerating said plant cell into a plant, thereby allowing the transcribed TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for producing a genome edited plant, which method comprises the steps of
a) simultaneously introducing an EDTGM protein and a TaWOX mRNA into a plant cell,
b) having the TaWOX mRNA transcribed in the cell,
c) allowing the EDTGM protein to modify the genome of said plant cell, and
d) regenerating said plant cell into a plant, thereby allowing the transcribed TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for producing a genome edited plant, which method comprises the steps of
a) simultaneously introducing an EDTGM mRNA and a TaWOX protein into a plant cell,
b) having the EDTGM mRNA transcribed in the cell,
c) allowing the transcribed EDTGM protein to modify the genome of said plant cell, and
d) regenerating said plant cell into a plant, thereby allowing the TaWOX protein to increase efficiency of regeneration and/or to shorten the regeneration time;
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

In a further embodiment, the present invention provides a method for producing a genome edited plant, which method comprises the steps of
a) simultaneously introducing an EDTGM protein and a mixture of TaWOX protein and TaWOX mRNA into a plant cell,
b) having the TaWOX mRNA transcribed in the cell,
c) allowing the EDTGM protein to modify the genome of said plant cell, and
d) regenerating said plant cell into a plant, thereby allowing the TaWOX protein (transcribed or introduced) to increase efficiency of regeneration and/or to shorten the regeneration time,
wherein the regeneration efficiency is increased compared to the regeneration of corresponding genome-edited plant cells without having said TaWOX introduced and/or wherein the regeneration time is decreased compared to the regeneration time of corresponding genome-edited plant cells without having said TaWOX introduced.

The methods of the invention are advantageously applicable to cells of any plant, in particular to any plant as defined herein. In a preferred embodiment, the cells are derived from a plant belonging to the Poaceae, more preferably the plant is a cereal, more preferably the plant is wheat. In a most preferred embodiment the plant cells are from an immature wheat embryo. The term “plant” as used herein encompasses whole plants, ancestors and progeny of the plants and plant parts, including seeds, shoots, stems, leaves, roots (including tubers), flowers, propagules, and tissues and organs. The term “plant” also encompasses plant cells, suspension cultures, callus tissue, embryos, meristematic regions, gametophytes, sporophytes, pollen and microspores. “Propagule” is any kind of organ, tissue, or cell of a plant capable of developing into a complete plant. A propagule can be based on vegetative reproduction (also known as vegetative propagation, vegetative multiplication, or vegetative cloning) or sexual reproduction. A propagule can therefore be seeds or parts of the non-reproductive organs, like stem or leave. In particular, with respect to Poaceae, suitable propagules can also be sections of the stem, i.e., stem cuttings. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to an embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, potato and tobacco. According to another embodiment of the present invention, the plant is a monocotyledonous plant. Examples of monocotyledonous plants include sugarcane. According to another embodiment of the present invention, the plant is a cereal. Examples of cereals include rice, maize, wheat, barley, millet, rye, triticale, sorghum, emmer, spelt, einkorn, teff, milo and oats. In a particular embodiment the plants of the invention, or used in the methods of the invention, are selected from the group consisting of maize, wheat, rice, soybean, cotton, oilseed rape including canola, sugarcane, sugar beet and alfalfa. The term “plant” as used herein encompasses whole plants, ancestors and progeny of the plants. Examples of plants of which the cells can be used in the methods of the present invention include Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The present invention also provides genome edited plants produced by the methods of the invention. Such plants can have improved agronomic traits. A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield. Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. A further important trait is that of improved abiotic stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible. Another trait of importance is resistance to biotic stress, typically caused by pathogens, such as bacteria, viruses, fungi, plants, nematodes and insects, or other animals, which may result in negative effects on plant growth.

Genome edited plants produced by the methods of the invention may also have improved nutritional traits such as higher content in protein, minerals, vitamins, micronutrients, essential amino acids levels or other health-promoting compounds.

The present invention extends further to encompass the progeny of the cell, tissue, organ or whole plant that has been produced by any of the aforementioned methods, the only requirement being that the progeny exhibit the same characteristics as the parent plant (i.e. at least the same genome modification and/or the same improved agronomic traits), provided however that the plant is produced by the methods of the invention and not by essentially biological processes, and provided that its progeny is different from, and can be discriminated from naturally occurring plants.

The invention also extends to harvestable parts of a genome edited plant produced by any of the aforementioned methods, such as, but not limited to seeds, leaves, fruits, flowers, stems, roots, rhizomes, tubers and bulbs, which harvestable parts comprise the desired edit or modification in the genome.

The invention furthermore relates to products derived or produced, preferably directly derived or directly produced, from a harvestable part of such a genome edited plant, such as dry pellets, pressed stems, meal or powders, oil, fat and fatty acids, carbohydrates, sap, juice or proteins. Preferred carbohydrates are starch, cellulose or sugars, preferably sucrose. Also preferred products are residual dry fibers, e.g., of the stem (like bagasse from sugar cane after cane juice removal), molasses, or filtercake for example from sugar cane. In one embodiment the product still comprises the genome or part thereof that comprises the desired edit or modification, which is useful for example as an indicator of the particular quality of the product. In another embodiment, the product derived or produced from a genome edited plant produced according to the invention is different from the product derived or produced from a plant not comprising the genome edit.

The invention also includes methods for manufacturing a product comprising a) growing the plants of the invention and b) producing said product from or by the genome edited plants of the invention or parts thereof, such as stem, root, leaves and/or seeds. In a further embodiment the methods comprise the steps of a) growing the plants of the invention, b) removing the harvestable parts as described herein from the plants and c) producing said product from, or with the harvestable parts of plants according to the invention.

In a further embodiment the products produced by the manufacturing methods of the invention are plant products such as, but not limited to, a foodstuff, feedstuff, a food supplement, feed supplement, fiber, cosmetic or pharmaceutical. In another embodiment the methods for production are used to make agricultural products such as, but not limited to, plant extracts, proteins, amino acids, carbohydrates, fats, oils, polymers, vitamins, and the like.

In yet another embodiment the nucleic acid comprising the desired edit or modification produced using the methods of the invention is comprised in an agricultural product. In a particular embodiment the nucleic acid comprising the desired edit or modification of the invention may be used as product marker, for example where an agricultural product was produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or anti-body based methods for protein detection.

The present invention also encompasses use of the nucleic acid comprising the desired edit or modification, it may find use in breeding programs in which a DNA marker is identified which may be genetically linked to the nucleic acid or locus on the genome comprising the desired edit or modification. The nucleic acid comprising the desired edit or modification itself may be used to define a molecular marker. This DNA marker may then be used in breeding programs to select plants having one or more enhanced yield-related traits as defined herein in the methods of the invention. Nucleic acids acid comprising the desired edit or modification may also be used as probes for genetically and physically mapping the genes that they are a part of, and as markers for traits linked to those genes. Such information may be useful in plant breeding in order to develop lines with desired phenotypes.

The present invention also provides a platform for producing genome edited plants, comprising a module for selecting the target gene for editing and designing a suitable gRNA that allows the desired modification of the target gene, a second module for performing the gene edit comprising preparation of the cells to be edited, introduction of the EDTGM together with a TaWOX, in the form of protein or mRNA according to the methods of the present invention, a third module for regenerating the edited cells into plants and a screening system for selecting the plants having the desired genome edit. The genome edited plants produced with this platform can subsequently be used in breeding programs.

### Non-Limiting List of Embodiments

I. A method for increasing the efficiency of regenerating a genome edited plant cell into a plant, comprising the steps of:
(a) simultaneously introducing into a plant cell (i) an EDTGM and (ii) a TaWOX, wherein said EDTGM and said TaWOX are introduced as protein or as its encoding mRNA;
(b) allowing the EDTGM to modify the genome of said plant cell;
(c) regenerating said plant cell into a plant, thereby allowing the introduced TaWOX to increase efficiency of regeneration;
wherein the regeneration efficiency is increased compared to the regeneration of corresponding genome-edited plant cells without having said TaWOX introduced.
   II. A method for increasing the speed of regenerating a genome edited plant cell into a plant, comprising the steps of:
(a) simultaneously introducing into a plant cell (i) an EDTGM and (ii) a TaWOX, wherein said EDTGM and said TaWOX are introduced as protein or as its encoding mRNA;
(b) allowing the EDTGM to modify the genome of said plant cell;
(c) regenerating said plant cell into a plant, thereby allowing the introduced TaWOX to increase the speed of regeneration;
wherein the regeneration efficiency is increased compared to the regeneration of corresponding genome-edited plant cells without having said TaWOX introduced.
   III. Method of embodiment I or II, wherein said TaWOX is a transcription factor comprising a Wuschel-like InterPro family IPR044555 domain.
   IV. Method of embodiment III, wherein the TaWOX further comprises a WUS-box motif and an ERF-associated amphiphilic repression motif.
   V. Method of embodiment III, wherein the TaWOX has at least 70%, 75%, 80%, 85%, 90%, 95%, 98%, 99% or more amino acid sequence identity to the homeobox domain of SEQ ID NO: 14, represented by SEQ ID NO: 29.
   VI. Method of embodiment I or II, wherein said TaWOX is a polypeptide comprising a sequence having in increasing order of preference at least 70%, 75%, 80%, 85%, 90%, 95%, 97% 97.2%, 97.5% 98%, 98.5%, 99%, 99.5%, 99.7%, 99.8%, 99.9% or more amino acid sequence identity to the TaWOX polypeptide represented by SEQ ID NO: 14.
   VII. Method of embodiment I or II, wherein said TaWOX is a polypeptide comprising the sequence represented by SEQ ID NO: 14.
   VIII. Method of embodiment I or II, wherein said TaWOX is a polypeptide with the sequence represented by SEQ ID NO: 14.
   IX. Method of embodiment I or II, wherein said TaWOX mRNA encodes a protein according to any of embodiments II to VIII.
   X. Method of embodiment III or IV, wherein said TaWOX is a protein substantially similar to the endogenous orthologue of SEQ ID NO: 14 naturally present in said genome edited plant cell.
   XI. Method of embodiment I or II, wherein said EDTGM is selected from: a CRISPR-Cas system, a Zn-finger endonuclease, a TALEN endonuclease, a meganuclease.
   XII. Method of embodiment I or II, wherein said EDTGM is a Class 2 CRISPR-Cas type endonuclease, preferably a type V or a type II CRISPR-Cas type endonuclease, more preferably a Cas9 or a Cas12 endonuclease, most preferably a Cas12a endonuclease derived from Acidaminococcus sp. BV3L6 or from Lachnospiraceae_bacterium_MC2017.
   XIII. Method of embodiment I or II, wherein said EDTGM mRNA encodes an EDTGM as defined herein, including, but not limited to, CRISPR-Cas variants such as modified Cas nuclease or nickases or inactivated Cas, or Cas fusion proteins.
   XIV. Method of embodiment I or II, wherein said simultaneous introduction into a cell is obtained by one of particle bombardment, electroporation, cationic lipid vesicles, nanoparticles, aerosol beam microinjection or sonoporation.
   XV. Method of embodiment I or II, wherein said increased regeneration efficiency is a higher percentage of cells regenerating into a plant, or a reduction of time needed for regeneration.
   XVI. Method of embodiment I or II, wherein said plant cell is derived from a plant recalcitrant to regeneration.
   XVII. Method for producing a genome edited plant comprising the steps of
(a) simultaneously introducing into a plant cell (i) an EDTGM and (ii) a TaWOX, wherein said EDTGM and said TaWOX are introduced as protein or as its encoding mRNA;
(b) allowing the EDTGM to modify the genome of said plant cell;
(c) regenerating said plant cell into a plant, thereby allowing the introduced TaWOX to increase efficiency of regeneration;
wherein the regeneration efficiency is increased compared to the regeneration of corresponding genome-edited plant cells without having said TaWOX introduced.
   XVIII. A plant produced by the methods of embodiment I, II or XVII, or a part of such plant.
   XIX. Progeny of the plant according to embodiment XVIII.
   XX. The plant part of embodiment XVIII, wherein said plant part is an harvestable part, an organ, or tissue.
   XXI. The plant of embodiment XVIII, wherein said plant is a monocotyledonous or a dicotyledonous plant, preferably fodder or forage legumes, ornamental plants, food crops, trees or shrubs.
   XXII. The plant according to embodiment XVIII, wherein said plant is a crop plant, preferably a cereal or a pulse or an oilseed plant, more preferably one of soybean, wheat, canola or cotton.
   XXIII. A method for manufacturing a product comprising the steps of growing the plants according to embodiment XVIII and producing said product from or by said plants; or parts thereof, including seeds.
   XXIV. Products obtained by the method of embodiment XXIII, which products are altered compared to the products of non-genome edited plants.
   XXV. A method for simultaneously delivering an EDTGM and a TaWOX into a plant cell, which method results in a desired targeted modification in the genome of the plant cell.
   XXVI. A particle designed for biolistic delivery into a cell, coated with a mixture of an EDTGM and a TaWOX, in the form of mRNA or protein.
   XXVII. Use of the particle of embodiment XXVI in a method for increasing the efficiency of genome editing of a plant cell.
   XXVIII. A method for increasing the efficiency of genome editing of a plant cell, and/or of decreasing the time required for genome editing of a plant cell comprising
a) simultaneously introducing into a plant cell (i) an EDTGM and (ii) a TaWOX, wherein said endonuclease and said TaWOX are introduced as protein or as its encoding mRNA;
b) allowing the EDTGM to modify the genome of said plant cell;
c) regenerating said plant cell into a plant, thereby allowing the introduced TaWOX to increase efficiency and/or speed of regeneration;
wherein the regeneration efficiency and/or speed is increased compared to the regeneration of corresponding genome-edited plant cells without having said TaWOX introduced.
   XXIX. Method of embodiment XXVIII, wherein said regenerated plant has improved agronomic and/or nutritional properties.
   XXX. A method for increasing the efficiency and/or speed of plant breeding comprising
a) simultaneously introducing into a plant cell (i) an EDTGM and (ii) a TaWOX, wherein said EDTGM and said TaWOX are introduced as protein or as its encoding mRNA;
b) allowing the EDTGM to modify the genome of said plant cell;
c) regenerating said plant cell into a plant, thereby allowing the introduced TaWOX to increase efficiency and/or speed of regeneration;
d) using said regenerated plant in a breeding process;
wherein the regeneration efficiency and/or speed is increased compared to the regeneration of corresponding genome-edited plant cells without having said TaWOX introduced.
   XXXI. Method for simultaneously delivering an EDTGM mRNA or the protein encoded thereby, and a TaWOX mRNA or the protein encoded thereby into a plant cell, which method results in a desired targeted modification in the genome of the plant cell.
   XXXII. Method according to any of embodiments I to VIII, wherein said TaWOX protein is fused to a peptide.
   XXXIII. Method according to any of embodiments I to VIII, wherein said TaWOX protein is fused to another protein, preferably to GFP.
   XXXIV. Method of embodiment I or II, wherein said TaWOX mRNA encodes a protein according to any of embodiments III to VIII, XXXII or XXXIII.
   XXXV. Method of any of embodiments I to VIII, wherein said TaWOX protein is part of a fusion protein, and wherein said fusion protein retains the same functionality as that of the protein represented by SEQ ID NO: 16.
   XXXVI. Method of embodiment XXXV, wherein said fusion protein has at least 90%, 95%, 96%, 97%, 97.5% 98%, 98.5%, 99%, 99.5%, 99.7%, 99.8%, 99.9% or more amino acid sequence identity to the TaWOX fusion polypeptide represented by SEQ ID NO: 16.
   XXXVII. Method of embodiment XXXV, wherein said fusion protein has at least 90%, 95%, 96%, 97%, 97.5% 98%, 98.5%, 99%, 99.5%, 99.7%, 99.8%, 99.9% or more amino acid sequence identity to the sequence spanning G211 to H471 in SEQ ID NO: 16.
   XXXVIII. Method of embodiment XXXV, wherein said fusion protein has at least 90%, 95%, 96%, 97%, 97.5% 98%, 98.5%, 99%, 99.5%, 99.7%, 99.8%, 99.9% or more amino acid sequence identity to the sequence spanning M1 to V210 in SEQ ID NO: 16.
   XXXIX. A method for increasing the efficiency of regenerating a genome edited plant cell into a plant, comprising the steps of:
(a) simultaneously introducing into a plant cell (i) an EDTGM and (ii) a functional fragment of a TaWOX, wherein said EDTGM and said functional fragment are introduced as protein or as its encoding mRNA;
(b) allowing the EDTGM to modify the genome of said plant cell;
(c) regenerating said plant cell into a plant, thereby allowing the introduced functional fragment to increase efficiency of regeneration;
wherein the functional fragment of a TaWOX has substantially the same biological activity of the full length TaWOX protein and wherein the regeneration efficiency is increased compared to the regeneration of corresponding genome-edited plant cells without having said TaWOX introduced.
   XL. A method for increasing the regeneration speed of a genome edited plant cell into a plant, comprising the steps of:
(d) simultaneously introducing into a plant cell (i) an EDTGM and (ii) a functional fragment of a TaWOX, wherein said EDTGM and said functional fragment are introduced as protein or as its encoding mRNA;
(e) allowing the EDTGM to modify the genome of said plant cell;
(f) regenerating said plant cell into a plant, thereby allowing the introduced functional fragment to increase the speed of regeneration;
wherein the functional fragment of a TaWOX has substantially the same biological activity of the full length TaWOX protein and wherein the regeneration speed is increased compared to the regeneration of corresponding genome-edited plant cells without having said TaWOX introduced.

### DESCRIPTION OF THE FIGURES

FIG. 1: Vector construct Pubi-wox5-3.1.2

FIG. 2: Vector construct Pubi-wox5-3.1.3-egfp

FIG. 3: Vector construct Pubi-egfp-wox5-3.1.1

FIG. 4: Vector construct P35S2-egfp-bar-1 Pe

FIG. 5: Cellular localization of GFP in the scutellum of bombarded wheat immature embryos (24 h after bombardment of DNA). A P35S2-egfp-bar-1 Pe, B Pubi-wox5-3.1.3-egfp and C Pubi-egfp-wox5-3.1.1.

FIG. 6: Morphogenic response from the scutellum of bombarded wheat immature embryos (4 days after bombardment of DNA). A P35S2-egfp-bar-1 Pe (negative control vector), B Pubi-wox5-3.1.2 (positive control vector), C Pubi-wox5-3.1.3-egfp and D Pubi-egfp-wox5-3.1.1.

FIG. 7: Histochemical visualization of GUS activity in the scutellum of bombarded wheat embryos (1 h after bombardment of GUS protein). A non-bombarded embryos (negative control 1), B embryos bombarded with GUS protein without gold (negative control 2), C embryos bombarded with GUS protein and gold.

FIG. 8: Amino acid sequence of TaWOX-eGFP-3C-6His protein (M.Wt.=53.26 kDa). Positions 1-210 (bold, underlined) TaWOX, positions 211-217=linker (italic), 218-455=eGFP (bold underlined), 456-463=3C protease site (italic), 464 471=6 His-tag and linker (bold).

FIG. 9: Morphogenic response from 6 individual wheat immature embryos bombarded with TaWOX-eGFP-3C-6His protein (3 weeks after delivery of the protein).

FIG. 10: Morphogenic response from 6 individual wheat immature embryos bombarded with GUS protein (3 weeks after delivery of the protein).

FIG. 11: Experiment TMTA0652: Morphogenic response of immature embryos bombarded with TaWOX-eGFP-3C-6His protein and controls without bombardment or bombarded only with gold. Average percentage response for embryos classified as i) without morphogenic structures, ii) producing 1-4 morphogenic structures, iii) producing 5 or more morphogenic structures. A Embryos bombarded with a rupture disk pressure of 450 psi, B Embryos bombarded with a rupture disk pressure of 650 psi.

FIG. 12: Experiment TMTA0660: Morphogenic response of immature embryos bombarded with TaWOX-eGFP-3C-6His protein and a control bombarded with gold and GUS protein. Average percentage response for embryos classified as i) without morphogenic structures, ii) producing 1-4 morphogenic structures, iii) producing 5 or more morphogenic structures. A Embryos bombarded with a rupture disk pressure of 450 psi, B Embryos bombarded with a rupture disk pressure of 650 psi.

FIG. 13: Experiment TMTA0674: Morphogenic response of immature embryos bombarded with TaWOX-eGFP-3C-6His protein and controls without bombardment or bombarded with gold and GUS protein. Average percentage response for embryos classified as i) without morphogenic structures, ii) producing 1-4 morphogenic structures, iii) producing 5 or more morphogenic structures. Embryos bombarded with a rupture disk pressure of of 650 psi.

FIG. 14: Experiment TMTA0721: Morphogenic response from immature embryos bombarded with TaWOX-eGFP-3C-6His protein and controls without bombardment or bombarded with gold and GUS protein (rupture disk pressure 450 psi). A Average percentage response of embryos classified as i) without morphogenic structures, ii) producing 1-4 morphogenic structures, iii) producing 5 or more morphogenic structures. B Average percentage response of embryos classified as i) without shoots, ii) producing 1-4 morphogenic shoots, iii) producing 5 or more shoots.

FIG. 15: Vector construct P-egfp-mRNA

FIG. 16: Fluorescent visualisation of GFP (2 h post bombardment) in non-bombarded control immature embryos (A) and immature embryos bombarded with GFP mRNA (B)

FIG. 17: Vector construct P-wox5-mRNA

FIG. 18: Multiple sequence alignment depicting the position of the Cas12a gRNA in exon 4 of the three wheat mlo homoeologues (MLO_5A: SEQ ID NO: 35, MLO_4B: SEQ ID NO: 36 and MLO_4D: SEQ ID NO: 37). The PAM site is shown boxed, the gRNA recognition site is underlined. Single nucleotide polymorphisms are shown in bold.

FIG. 19: Schematic drawing (SEQ ID NO: 38) of the ddPCR design indicating the location, orientation and sequences of the different probes and primers. The position of the predicted cut site by Cas12a and the R158Q edit are also indicated. The edit probe only binds to alleles modified by HDR, whereas the drop-off probe loses its binding site when insertions or deletions are created by NHEJ.

FIG. 20: Validation of the ddPCR assay by NGS analysis

FIG. 21: ddPCR assay sensitivity established by serial dilution of HDR and NHEJ synthetic templates in a constant background (200 ng) of WT genomic DNA. The limit of detection was ˜0.1% for NHEJ and ˜0.04% for HDR, as determined by comparison with WT genomic DNA controls. Data are means ±standard deviation of two merged wells per dilution and three merged wells for the WT control.

FIG. 22: Plate of RNP bombarded immature wheat embryos indicating the positions of Zone 1 (centre target area), Zone 2 (inner circle target area) and Zone 3 (outer circle target area).

FIG. 23: Process for leaf sampling of pools from PlantCon™ containers: Shoots regenerated from immature embryo derived callus (A), open-container showing shoots with long leaves (B), container with shoots pre-trimmed prior to sampling (C), container with shoots from which uniform leaf samples are sampled (D), dish with sampled pooled leaf explants (E) and container ready for shoot regrowth and 6 ml sample tube (F).

FIG. 24: Overview of the complete 3-step selection system to identify RNP-induced indels in wheat plants.

FIG. 25: A detailed scheme of the selection/analysis process for identifying Cas12a RNP-induced indels in wheat plants, with timelines and processes from embryo RNP bombardment to transfer of structured embryogenic callus tissue to PlantCon™ containers. The time point for sampling 1 is indicated in bold.

FIG. 26: A detailed scheme of the selection/analysis process for identifying Cas12a RNP-induced indels in wheat plants, with timelines and processes from transfer of structured embryogenic callus tissue to PlantCon™ containers to transfer of mutant plants to the greenhouse. The time points for sampling 2 and sampling 3 are indicated in bold.

### EXAMPLES SECTION

### Example 1 Confirmation of the Efficacy of TaWOX-GFP and GFP-TaWOX Fusion Constructs for Enhanced Regeneration

Preliminary protein expression studies (ProteoGenix, Schiltigheim, France) showed that TaWOX protein purification under native conditions the addition of an eGFP tag was required. Purification of TaWOX under denaturing conditions requires an additional refolding, negatively affecting the function of the protein and the yields during purification.

To test if eGFP tagged TaWOX retains its activity as a fusion protein, constructs with both C- and N-terminal eGFP were designed and tested along with suitable controls in wheat immature embryos.

### Vector Construction

### Pubi-wox5-3.1.2

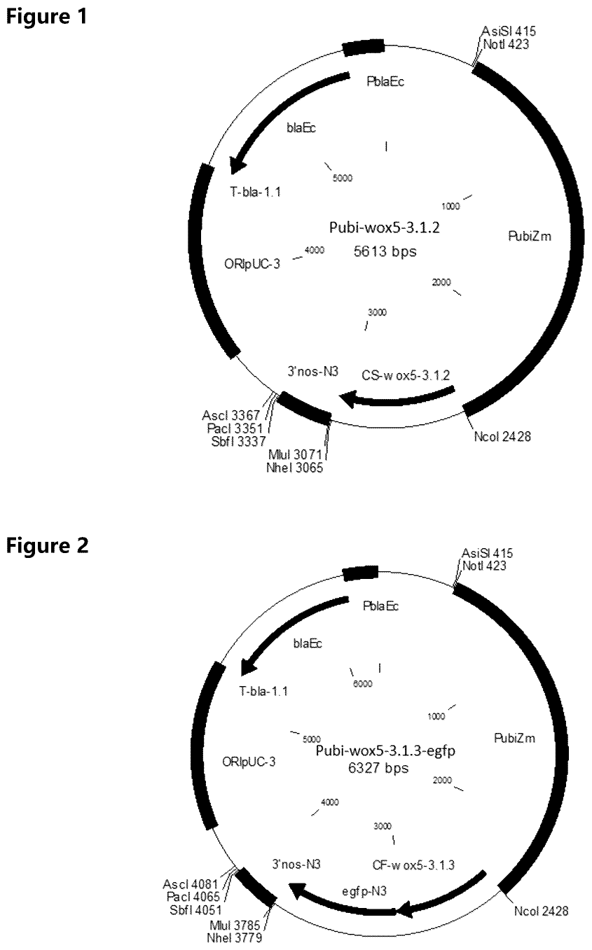

Vector containing the coding sequence of the WUSCHEL-related homeobox 5 gene from Triticum aestivum, cv. Chinese Spring (subgenome D) under control of the constitutive maize ubiquitin promoter (PubiZm). The TaWOX coding sequence (SEQ ID NO:13, Accession number: Dbase TRIAE_CS_IWGSC_CDS_1_1: TraesCS3D02G361100.1) was optimized for wheat using proprietary ‘GeneOptimizer’ sequence software (parameter settings=high expression wheat genes codon usage and 56% GC). For Pubi-wox5-3.1.2 (FIG. 1) the optimized sequence CS-wox5-3.1.2, was made synthetically as a gBlock (IDT-DNA) and cloned using standard molecular cloning methods into a pUC-derived cloning vector together with the maize Ubiquitin 1 gene promoter (Christensen et al., 1992) and the 3′ UTR of the nopaline synthase gene from the T-DNA of pTiT37 (Depicker et al., 1982) containing the polyadenylation signal.

Pubi-wox5-3.1.3-egfp

Vector containing the coding sequence of the enhanced green fluorescent protein gene of Aequorea victoria fused with the WUSCHEL-related homeobox 5 gene from Triticum aestivum, cv. Chinese Spring (subgenome D) under control of the constitutive maize ubiquitin promoter. For Pubi-wox5-3.1.3-egfp (FIG. 2) a PCR derived fragment of the egfp gene (Cormack et al., 1996), missing its start codon, was inserted at the 3′ end of the TaWOX gene in Pubi-wox5-3.1.2, creating a seamless TaWOX>egfp fusion coding sequence.

Pubi-egfp-wox5-3.1.1

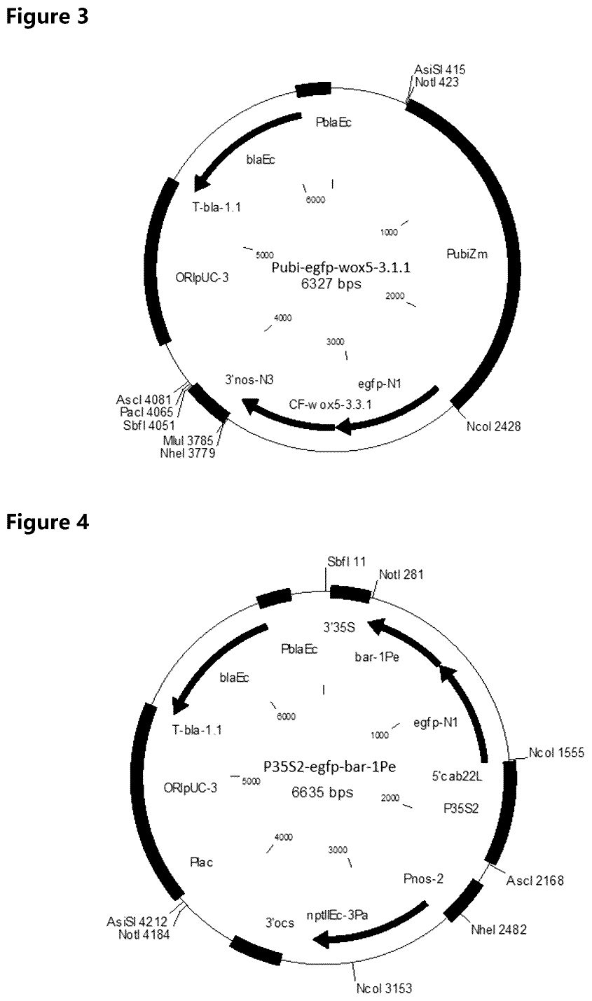

Vector containing the coding sequence of the enhanced green fluorescent protein gene of Aequorea victoria fused with the WUSCHEL-related homeobox 5 gene from Triticum aestivum, cv. Chinese Spring (subgenome D) under control of the constitutive maize ubiquitin promoter. For Pubi-egfp-wox5-3.1.1 (FIG. 3) a PCR derived fragment of the egfp gene (Cormack et al., 1996), missing its stop codon, was inserted at the 5′ end of the TaWOX gene to remove the start codon in Pubi-wox5-3.1.2, creating a seamless egfp>TaWOX fusion coding sequence.

P35S2-egfp-bar-1 Pe

Control vector containing the coding sequence of the enhanced green fluorescent protein gene of Aequorea victoria fused with the phosphinothricin acetyltransferase gene of Streptomyces hygroscopicus under control of the constitutive 35S cauliflower mosaic virus promoter. For P35S2-egfp-bar-1 Pe (FIG. 4) the egfp gene (Cormack et al., 1996), bar gene including start codon (Thompson et al., 1987), promoter region of the 35S transcript gene of cauliflower mosaic virus (Odell J T. et al., 1985), 5′ UTR of the chlorophyl a/b binding protein gene of Petunia x hybrida (Harpster et al., 1988) and the 3′ UTR of the octopine synthase gene of Agrobacterium tumefaciens (Sanfaç on et al., 1991) containing the polyadenylation signal were cloned together into a pUC-derived cloning vector using standard molecular cloning methods.

Vector constructs were verified by restriction enzyme analysis and Sanger sequencing. Large scale, endotoxin-free plasmid preparation was performed using the ZymoPURE II Plasmid Maxiprep Kit (Zymo Research Corp., cat no D4203) according to manufacturer's instructions. Plasmid DNA preparations were adjusted to final concentrations 1 μg/μl in TE buffer (pH 8.0) and stored in a freezer at −20° C.

### Wheat Donor Plant Growth for Immature Embryo Isolation

Donor plants of the spring wheat cv. Fielder (cv. HR1) were grown under controlled-environment conditions to ensure optimal immature embryo quality. Typical growth conditions were day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 9 cm Petri dishes containing 35-40 ml of non-selective callus induction medium WLS. Embryos were arranged in a central circle of 1.5-2 cm (approximately 50 embryos/dish with the scutellum side facing upwards).

### Biolistic Delivery of DNA

DNA delivery into immature embryos was conducted using the Biolistic® PDS-1000/He Particle Delivery System (Bio-Rad) essentially as described by Sparks and Jones (2014). Plasmid DNA was coated onto 0.6 μm gold particles (BioRad), aliquots spread over the central region of each macrocarrier and airdried prior to delivery. For each shot approximately 150 μg of gold particles carrying 300 ng of plasmid DNA were delivered with a pressure of 1350 psi with a target distance of approximately 6 cm.

### Cellular Localization of GFP in Bombarded Wheat Immature Embryos

Following bombardment, immature embryos were incubated on the same plates for 24 h in the dark (250C+/−1° C., 55% relative humidity in an MLR-352H-PE Panasonic incubator). Functionality of eGFP in Pubi-wox5-3.1.3-egfp and Pubi-egfp-wox5-3.1.1 was then visualized using a stereo microscope (SZX16, Olympus) equipped with a fluorescence illumination system (X-Site® Series 120 Q, Excelitas) and GFP/YFP filter sets (sZX2-FGFP/SZX2-FYFPHQ, Olympus). Images were captured using a digital camera (U31SPM Series C-mount USB3.0 CMOS, Touptek) controlled with image processing software (Toupview).

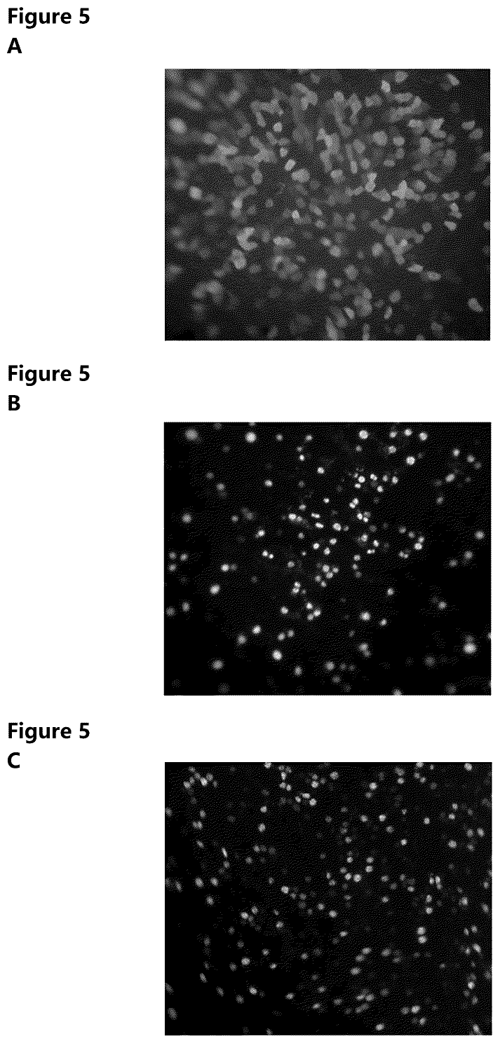

Bombarded immature embryos, using Pubi-wox5-3.1.3-egfp or Pubi-egfp-wox5-3.1.1 and the control GFP construct P35S2-egfp-bar-1Pe showed multiple GFP positive cells over the complete target area. However, in contrast to the control vector P35S2-egfp-bar-1 Pe, which showed cytoplasmic expression (FIG. 5A), both Pubi-wox5-3.1.3-egfp and Pubi-egfp-wox5-3.1.1 GFP was localized to the nuclei (FIGS. 5B and C).

Evaluation of TaWOX-GFP and GFP-TaWOX Fusions for Improved Regeneration Response from Bombarded Wheat Immature Embryos

Twenty-four hours after bombardment immature embryos were transferred to non-selective callus induction medium 240 (9 cm dishes containing 35-40 ml of medium with up to 15 embryos/dish). The developmental response of the embryo scutellum was assessed after a further 3 days of culture using a stereo microscope (SZX16, Olympus) and images captured using a digital camera (U31SPM Series C-mount USB3.0 CMOS, Touptek) controlled with image processing software (Toupview).

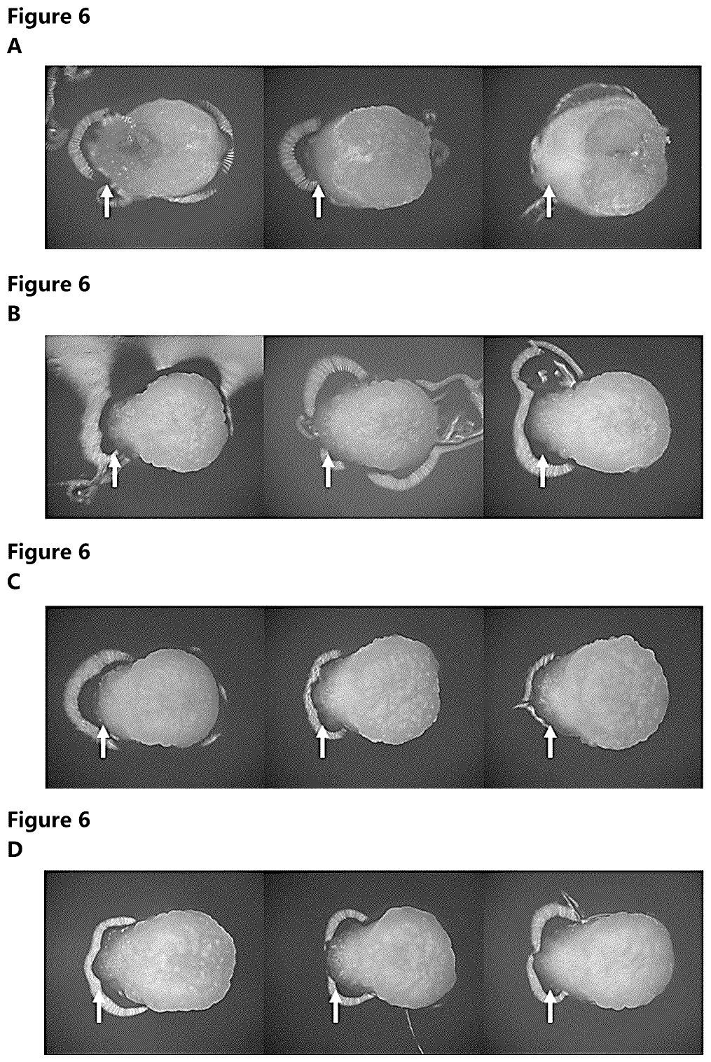

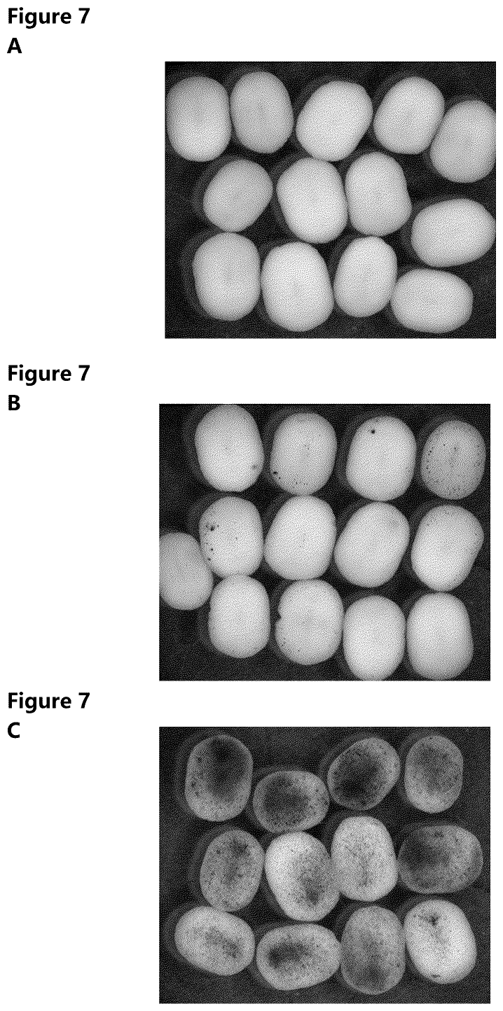

The scutellum of embryos bombarded with P35S2-egfp-bar-1Pe (negative control vector) developed soft, watery callus with a limited number of morphogenic structures (FIG. 6A), whereas embryos bombarded with Pubi-wox5-3.1.2 (positive control vector) developed a compact, morphogenic structured scutellum, even at the coleoptilar-tip of the scutellum, which typically shows a limited response (FIG. 6B).

The scutellum of embryos bombarded with Pubi-wox5-3.1.3-egfp or Pubi-egfp-wox5-3.1.1 also developed structures comparable to the positive control vector Pubi-wox5-3.1.2 (FIGS. 6C and D), confirming activity of TaWOX in the fusion proteins, however the observed response was more efficient using Pubi-wox5-3.1.3-egfp.

### Example 2 Optimization of Conditions for Biolistic Delivery of Protein

### Wheat Donor Plant Growth for Immature Embryo Isolation

Donor plants of cv. Fielder spring wheat were grown under controlled-environment conditions to ensure optimal immature embryo quality. Typical growth conditions were day/night temperatures of 200 C+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

### Biolistic Delivery of GUS Protein

Purified GUS protein (β-Glucuronidase from E. coli, Sigma-Aldrich) was used as a reporter protein to establish conditions for biolistic protein delivery into scutellum cells of immature wheat embryos.

For 4 shots, 16 μg of GUS protein, 6 μl protease inhibitor (cOmplete™ ULTRA Tablets, Mini, EASYpack Protease Inhibitor Cocktail, Roche prepared as a 10× stock solution) were mixed with 600 μg of 0.6 μm gold particles (BioRad) and the volume adjusted finally to 60 μl with sterile water. Aliquots of 15 μl of the mix were spread over the central region of each macrocarrier, airdried in a laminar flow and delivered using the Bio-Rad PDS-1000/He Particle Delivery System with a pressure of 450/650 psi with a target distance of approximately 3 cm.

Control bombardments were also made with GUS protein, delivered as previously described, but without gold particles.

Following bombardment, immature embryos were incubated on the same plates in the dark (25° C.+/−1° C., 55% relative humidity in an MLR-352H-PE Panasonic incubator).

### Evaluation of GUS Protein Activity in Bombarded Embryos

Approximately 1 hour after bombardment the embryos were collected in a 2.0 ml Eppendorf tube, washed several times with mild vortexing in liquid WLS medium (Ishida et al., 2015) and histochemically stained to detect GUS activity (Jefferson et al., 1987). Embryos were incubated in aliquots of X-Gluc staining solution for 16 h at 37° C. in the dark.

GUS activity in embryo cells was evaluated using a stereo microscope (SZX16, Olympus) and images captured using a digital camera (U3ISPM Series C-mount USB3.0 CMOS, Touptek) controlled with image processing software (Toupview). Representative images from i) non-bombarded embryos, ii) embryos bombarded with GUS protein without gold and iii) bombarded with GUS protein and gold are shown (FIG. 7). No evidence for GUS activity was detected in the non-bombarded embryo samples (FIG. 7A). In the embryos that were bombarded with protein without gold, GUS activity was detected only in a limited number of cells (FIG. 7B). However, in the embryos bombarded with protein and gold a very large number of cells showed GUS activity (FIG. 7C). Collectively these observations provide strong evidence that conditions for efficient protein delivery into immature embryos have been established.

### Example 3 TaWOX-eGFP-3C-6his Protein Production and Purification

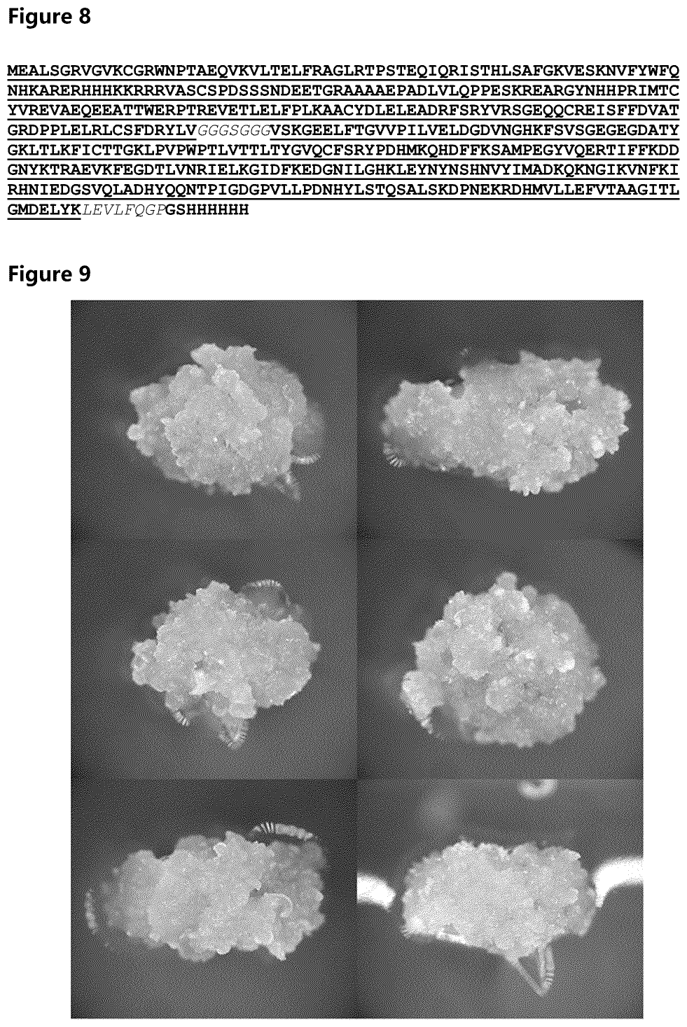

The TaWOX-eGFP-3C-6His coding sequence was synthesized, subcloned into an expression vector and the protein produced and purified from E. coli using the expression platform of ProteoGenix (Schiltigheim, France). The amino acid sequence of TaWOX-eGFP-3C-6His was complemented with a 6 His-tag for purification and a 3C protease site (FIG. 8), although this cleavage site was not used. The TaWOX-eGFP-3C-6His protein was purified in native conditions and thereafter dialyzed and concentrated by ultrafiltration. The TaWOX-eGFP-3C-6His was provided as a 2 μg/μl stock in TBS pH 8 (glycerol 50% v/v).

### Example 4 Biolistic Delivery of TaWOX-eGFP-3C-6his Protein for Enhanced Regeneration

### Wheat Donor Plant Growth

Donor plants of a highly recalcitrant spring wheat (cv. HR1) were grown under controlled-environment conditions to ensure optimal immature embryo quality. Typical growth conditions were day/night temperatures of 200 C+/−10C and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

### Biolistic Delivery of TaWOX-eGFP-3C-6his Protein

Purified TaWOX-eGFP-3C-6His protein was biolistically delivered into immature embryos using the Biolistic®PDS-1000/He Particle Delivery System (Bio-Rad).

For 10 shots, 40 μg of TaWOX-eGFP-3C-6His protein, 15 μl protease inhibitor (cOmplete™ ULTRA Tablets, Mini, EASYpack Protease Inhibitor Cocktail, Roche prepared as a 10× stock solution) were mixed with 1.65 mg of 0.6 μm gold particles (BioRad) and the volume adjusted finally to 150 μl with sterile water. Aliquots of 15 μl of the mix were spread over the central region of each macrocarrier, airdried for 60 min in a laminar flow bench and delivered with a pressure of 450/650 psi with a target distance of approximately 3 cm.

Controls included: i) non-bombarded embryos, ii) embryos bombarded with only gold particles and iii) embryos bombarded with gold particles and purified GUS protein.

Following bombardment, the immature embryos were incubated on the same plates for 24 h in the dark (28° C.+/−1° C., 55% relative humidity in an MLR-352H-PE Panasonic incubator).

### Assessment of TaWOX-eGFP-3C-6his Protein Activity for Enhanced Regeneration

Four independent experiments were performed delivering TaWOX-eGFP-3C-6His protein to immature embryos of highly recalcitrant spring wheat (cv. HR1). Twenty-four hours after bombardment the embryos were transferred to fresh non-selective callus induction medium 240 (9 cm dishes containing 35-40 ml of medium with up to 15 embryos/dish) and incubated in the dark (25° C.+/−1° C.).

Fourteen days later the immature embryos were longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

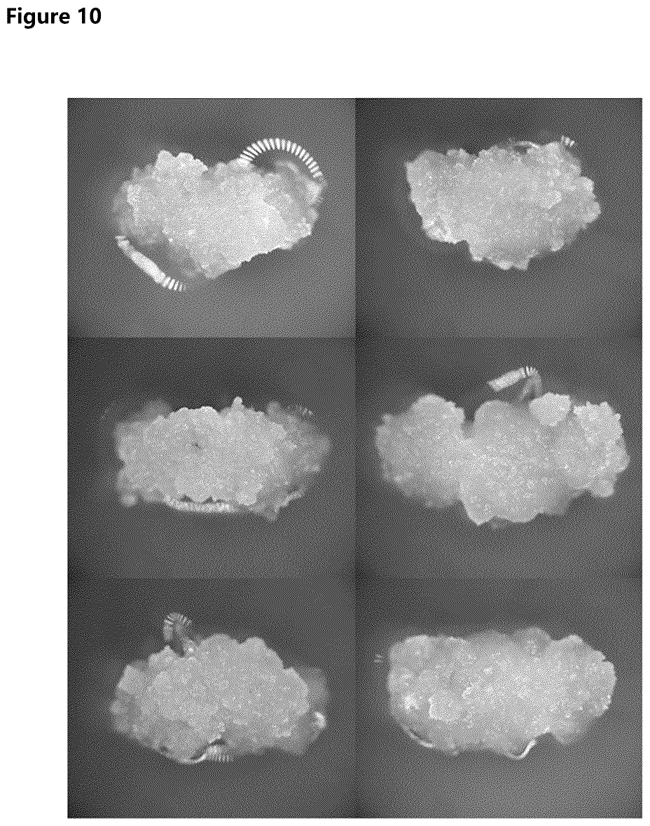

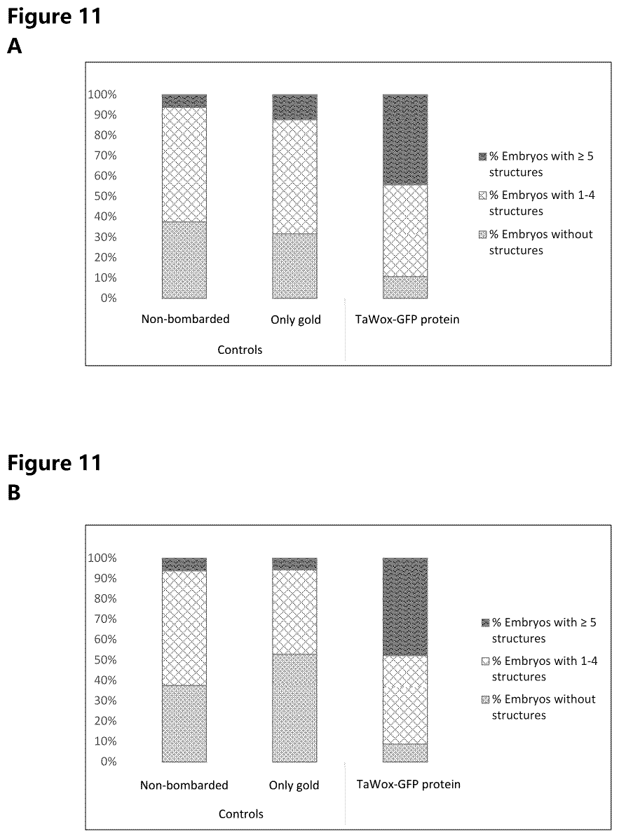

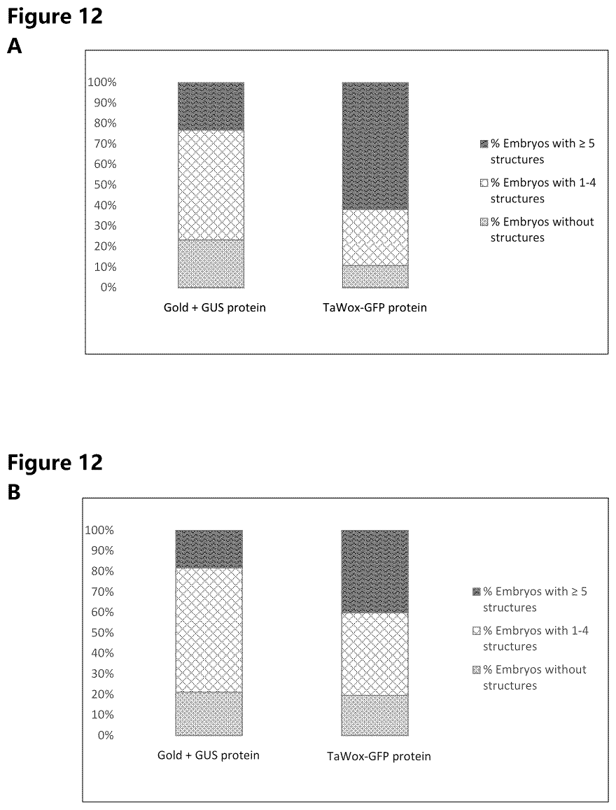

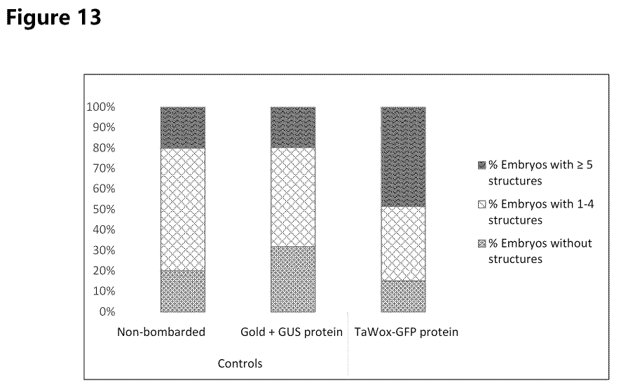

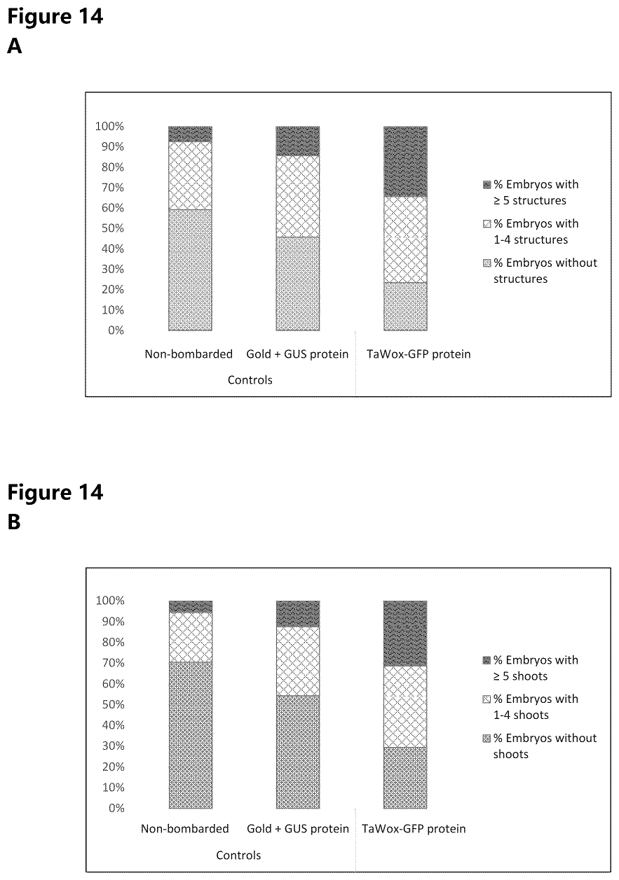

After a further 3 weeks culture, the bombarded embryos from each independent experiment (Experiment 1 TMTA0652, Experiment 2 TMTA0660, Experiment 3 TMTA0674 and Experiment 4 TMTA0721) were evaluated for morphogenic response. The scutellum of embryos bombarded with TaWOX-eGFP-3C-6His protein (FIG. 9) generally showed a higher morphogenic response compared with non-bombarded controls and controls bombarded with just gold or gold and GUS protein (FIG. 10). For each embryo the number of embryogenic structures was determined using a stereo microscope (SZX16, Olympus). To simplify the evaluation, individual embryo response was classified into one of 3 categories: i) embryos without any embryogenic structures, ii) embryos with 1-4 embryogenic structures and iii) embryos with 5 embryogenic structures. For each delivery type, counts were made for 3 replicate shots and average values calculated. The overall results for Experiment 1 TMTA0652 (FIGS. 11A and B), Experiment 2 TMTA0660 (FIGS. 12A and B), Experiment 3 TMTA0674 (FIG. 13) and Experiment 4 TMTA0721 (FIG. 14A) showed the the percentage of embryos developing 5 embryogenic structures was consistently higher than in any of the 3 different controls (non-bombarded controls and controls bombarded with just gold or gold and GUS protein).

To investigate shoot production, pieces of structured embryogenic callus tissue from Experiment 4 TMTA0721 were transferred to a non-selective regeneration medium, LSZ (Ishida et al., 2015), designated medium 420, and incubated in the light for 4-6 weeks (23° C.+/−1° C., 16 h photoperiod). In agreement with the observations made at the callus level, consistently more shoots/embryo were recovered from bombardments with TaWOX-eGFP-3C-6His protein compared to controls (FIG. 14B).

### Example 5 Optimization of Conditions for Biolistic Delivery of mRNA

GFP mRNA Preparation

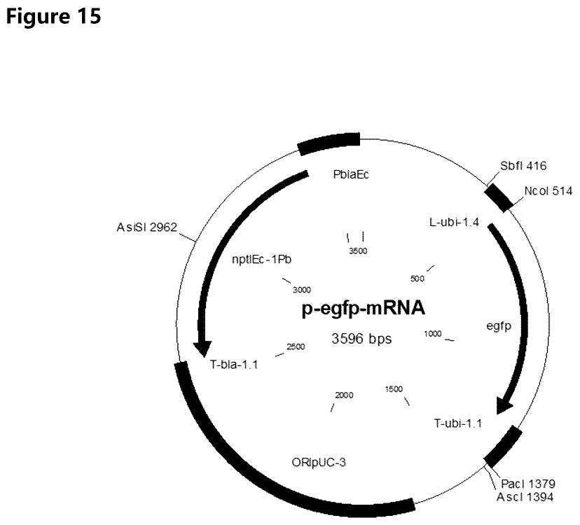

The egfp gene coding sequence (Cormack et al., 1996), flanked by 5′ and 3′ UTR sequences originating from the maize Ubiquitine 1 gene (Christensen et al., 1992) were synthetically made as a gBlock ds DNA fragment (IDT-DNA) and cloned into a pUC57-Kan cloning vector using standard molecular cloning techniques to generate vector P-eGFP-mRNA (FIG. 15). The resulting vector was then used with primers (Seq ID NO: 30/Seq ID NO: 31), to produce a PCR product serving as a template for RNA in vitro transcription. A T7 promoter was included in the 5′ end of the forward PCR primer (Seq ID NO: 30). eGFP mRNA was in vitro transcribed using a HiScribe T7 ARCA mRNA Kit (with tailing) cat. No NEB #E2060S (New England Biolabs) according to manufacturer's instructions. A DNAse step was included for the removal of the DNA template. The resulting ARCA capped (5′) and poly(A) tailed egfp mRNA was purified using a Monarch RNA Cleanup Kit Cat. no NEB #T2050S/L (New England Biolabs) and eluted into 50 μl of nuclease-free water (end concentration approximately 1 μg/μl). The mRNA integrity was confirmed before and after tailing using an Agilent 2100 Bioanalyzer with an RNA 6000 Nano assay chip according to manufacturer's instructions.

### Wheat Donor Plant Growth for Immature Embryo Isolation

Donor plants of a highly recalcitrant spring wheat (cv. HR1) were grown under controlled-environment conditions to ensure optimal immature embryo quality. Typical growth conditions were day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds were harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds were washed several times with demineralized water.

Embryos were aseptically excised from the immature seeds using a binocular microscope (Model MZ6, Leica) taking care to slice-off the embryo axis through the green seed coat during preparation. Subsequent steps for embryo preparation were performed using a modified procedure essentially as described by Ishida et al. (2015). Immature embryo explants were transferred to 3.5 cm Petri dishes (Falcon 351008) containing 4.5 ml of non-selective callus induction medium WLS, herewith designated callus induction medium 240. Embryos were arranged in a central circle of 1.5 cm (25-50 embryos/dish with the scutellum side facing upwards).

Biolistic Delivery of GFP mRNA

For 10 shots, 10 μg of GFP mRNA, 20 μl of mRNA Boost reagent (TransIT-mRNA transfection kit, Mirus) and 20 μl of TransIT-mRNA reagent (TransIT-mRNA transfection kit, Mirus) were mixed with 1.65 mg of 0.6 μm gold particles (BioRad) and the volume adjusted finally to 150 μl with sterile water. The mixture was incubated at room temperature for 2-3 min and then centrifuged in an Eppendorf MiniSpin centrifuge (12,000 rpm, 10 sec). After removal of the supernatant the pellet was resuspended in 150 μl ethanol by brief sonication (37 kHz, 50% power in sweep mode, Elmasonic P Sonicator). Aliquots of 15 μl of the mix were spread over the central region of each macrocarrier, airdried in a laminar flow and delivered using the Bio-Rad PDS-1000/He Particle Delivery System with a pressure of 450 psi with a target distance of approximately 3 cm.

Following bombardment, immature embryos were incubated in the dark (25° C.+/−1° C., 55% relative humidity in an MLR-352H-PE Panasonic incubator) and GFP expression visualized using a stereo microscope (SZX16, Olympus) equipped with a fluorescence illumination system (X-Site® Series 120 Q, Excelitas) and GFP/YFP filter sets (sZX2-FGFP/SZX2-FYFPHQ, Olympus). Images were captured at defined intervals using a digital camera (U3ISPM Series C-mount USB3.0 CMOS, Touptek) controlled with image processing software (Toupview).

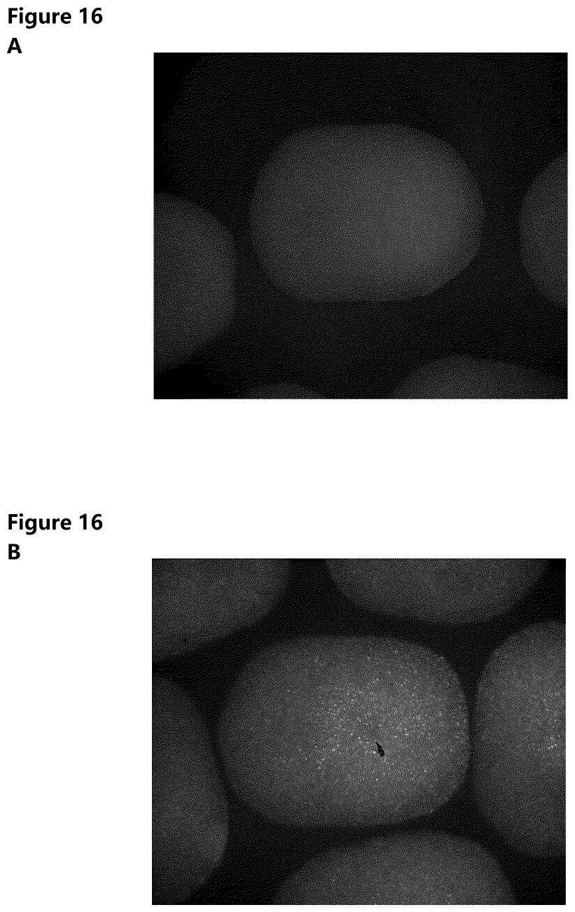

In immature embryos bombarded with GFP mRNA, GFP accumulated to detectable levels approximately 2 hours after bombardment (FIG. 16B), indicating the functional delivery and expression of mRNA in bombarded wheat scutellum cells.

In contrast, control immature embryos (non-bombarded) showed no detectable GFP expression (FIG. 16A).

### Example 6 Biolistic Delivery of Tawox mRNA for Enhanced Regeneration from Wheat Immature Embryos

TaWox mRNA Preparation

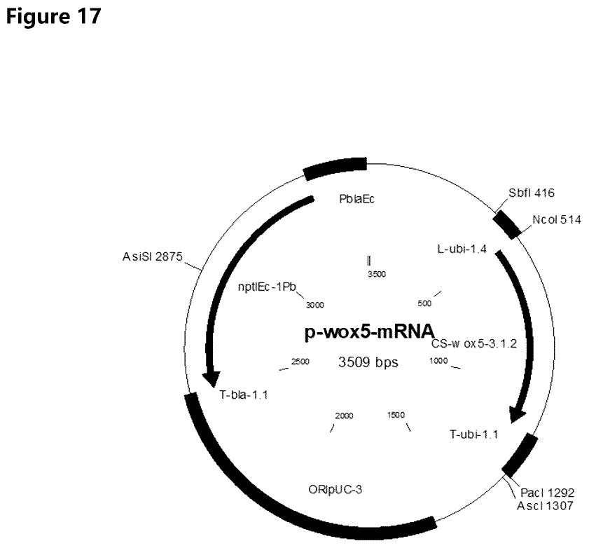

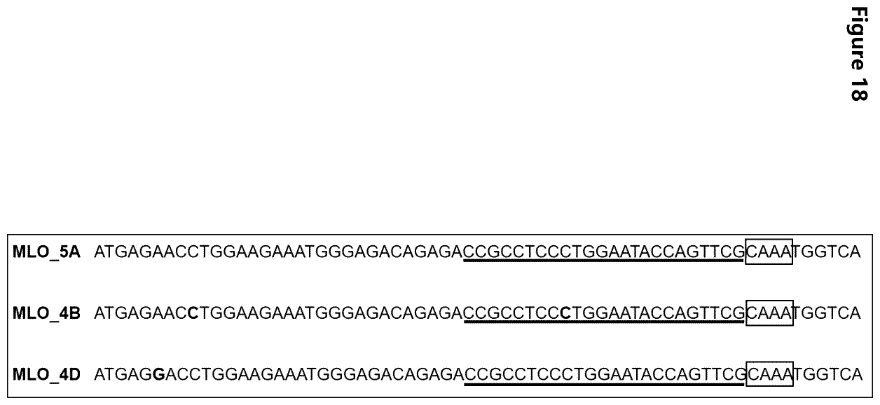

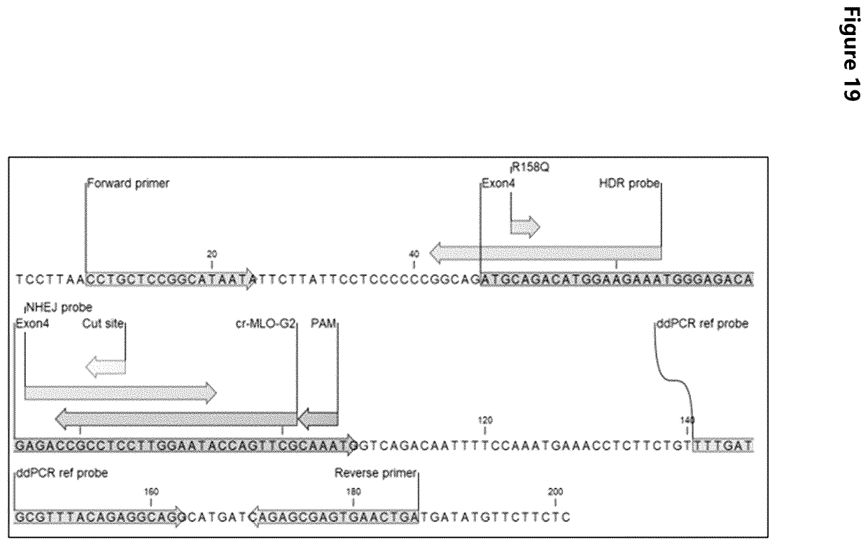

The TaWox gene coding sequence (from vector Pubi-wox5-3.1.2), flanked by 5′ and 3′ UTR sequences originating from the maize Ubiquitine 1 gene (Christensen et al., 1992) is synthetically made as a gBlock ds DNA fragment (IDT-DNA) and cloned into a pUC57-Kan cloning vector using standard molecular cloning techniques to generate vector P-Wox5-mRNA (FIG. 17). The resulting vector was then used with primers (Seq ID NO: 30/Seq ID NO: 31), to produce a PCR product serving as a template for RNA in vitro transcription. A T7 promoter was included in the 5′ end of the forward PCR primer (Seq ID NO: 30). TaWox mRNA is in vitro transcribed using a HiScribe T7 ARCA mRNA Kit (with tailing) cat. No NEB #E2060S (New England Biolabs) according to manufacturer's instructions. A DNAse step is included for the removal of the DNA template. The resulting ARCA capped (5′) and poly(A) tailed egfp mRNA is purified using a Monarch RNA Cleanup Kit Cat. no NEB #T2050S/L (New England Biolabs) and eluted into 50 μl of nuclease-free water (end concentration approximately 1 μg/μl). The mRNA integrity is confirmed before and after tailing using an Agilent 2100 Bioanalyzer with an RNA 6000 Nano assay chip according to manufacturer's instructions.

### Wheat Donor Plant Growth for Immature Embryo Isolation

Donor plants of a highly recalcitrant spring wheat (cv. HR1) are grown under controlled-environment conditions to ensure optimal immature embryo quality. Typical growth conditions are day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds are harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds are washed several times with demineralized water.

Embryos are aseptically excised from the immature seeds using a binocular microscope (Model MZ6, Leica) taking care to slice-off the embryo axis through the green seed coat during preparation. Subsequent steps for embryo preparation are performed using a modified procedure essentially as described by Ishida et al. (2015). Immature embryo explants are transferred to 3.5 cm Petri dishes (Falcon 351008) containing 4.5 ml of non-selective callus induction medium WLS, herewith designated callus induction medium 240. Embryos are arranged in a central circle of 1.5 cm (25-50 embryos/dish with the scutellum side facing upwards).

Biolistic Delivery of TaWox mRNA

For 10 shots, 10 μg of TaWox mRNA, 20 μl of mRNA Boost reagent (TransIT-mRNA transfection kit, Mirus) and 20 μl of TransIT-mRNA reagent (TransIT-mRNA transfection kit, Mirus) are mixed with 1.65 mg of 0.6 μm gold particles (BioRad) and the volume adjusted to 150 μl with sterile water. The mixture is incubated at room temperature for 2-3 min and centrifuged in an Eppendorf MiniSpin centrifuge (12,000 rpm, 10 sec). After removal of the supernatant the pellet is resuspended in 150 μl ethanol by brief sonication (37 kHz, 50% power in sweep mode, Elmasonic P Sonicator). Aliquots of 15 μl of the mix are spread over the central region of each macrocarrier, airdried in a laminar flow and delivered using the Bio-Rad PDS-1000/He Particle Delivery System with a pressure of 450 psi with a target distance of approximately 3 cm.

Control bombardments are performed as described above but without addition of the TaWox mRNA.

Following bombardment, immature embryos are incubated in the dark (25° C.+/−1° C., 55% relative humidity in an MLR-352H-PE Panasonic incubator). Twenty-four hours after bombardment the embryos are transferred to fresh non-selective callus induction medium 240 (9 cm dishes containing 35-40 ml of medium with up to 15 embryos/dish) and incubated in the dark (250C+/−1° C.).

Fourteen days later the immature embryos are longitudinal bisected into 2 pieces under a binocular microscope and transferred to fresh non-selective callus induction medium 240 (9 cm dishes containing 35-40 ml of medium, 12 embryos/dish) and cultured in the dark (250C+/−1° C.).

After a further 3 weeks culture, the bombarded embryos are evaluated for morphogenic response. The scutellum of embryos bombarded with TaWox mRNA showed a higher morphogenic response compared to controls bombarded with gold and no mRNA, indicating the functional delivery and expression of TaWox mRNA in bombarded wheat scutellum cells.

To investigate total shoot production, pieces of structured embryogenic callus tissue are transferred to a non-selective regeneration medium, LSZ (Ishida et al., 2015), designated medium 420, and incubated in the light for 4-6 weeks (23° C.+/−1° C., 16 h photoperiod). In agreement with the observations made at the callus level, consistently more shoots/embryo are recovered from bombardments with TaWox mRNA compared to controls.

### Example 7 Co-Delivery of TaWOX-eGFP-3C-6his Protein and CRISPR Cas12a RNP for Enhanced Recovery of Mlo Mutants in Wheat

qRNA Design for Cas12a Targeting of Mlo

Building upon previous work (Gil-Humanes et al. 2017) we designed a synthetic crRNA composed of a 21 bp direct repeat sequence and a 24 bp protospacer targeting the wheat mildew resistance locus O (mlo). The mlo gene encodes a seven-transmembrane domain protein involved in resistance to the fungal pathogen Blumeria graminis. The recognition site of the crRNA is located on the antisense strand within exon 4 of mlo [5′-[(TTTG)CGAACTGGTATTCCAAGGAGGCGG-3′], with PAM site between brackets (SEQ ID NO: 34). The designed guide is specific for the 5A and 4D alleles of mlo and shows one mismatch with the 4B allele at position 16 from the PAM sequence (FIG. 18).

Design of a Droplet Digital PCR (ddPCR) Assay to Detect Cas12a RNP-Induced Mloindel Mutations

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

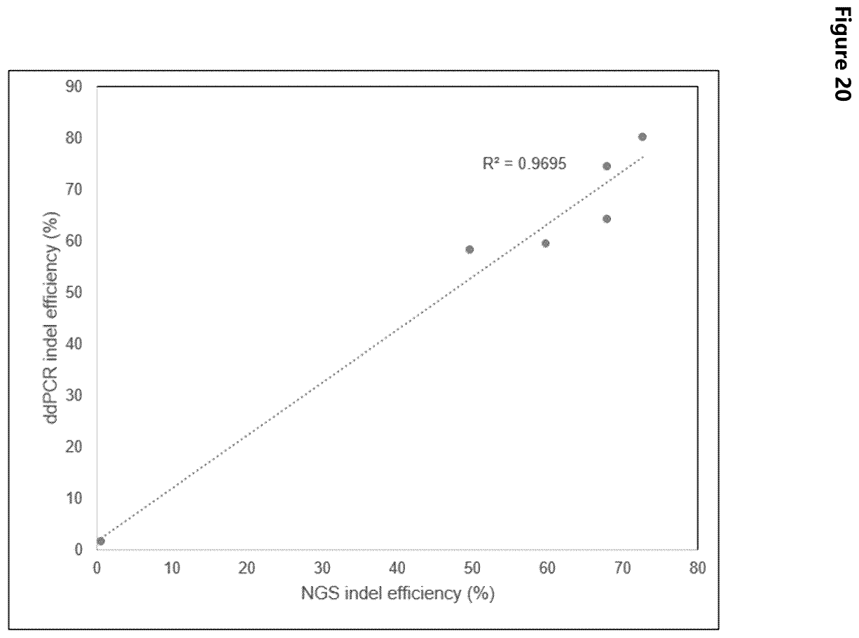

The ddPCR assay used to identify Cas12a-RNP induced mlo indels was the same as that previously designed for the detection of Cas9-RNP induced mlo indels (as described below). The location of the predicted Cas12a cut site in the mlo gene and the position of the PCR primers and probes used for the ddPCR analysis are shown in FIG. 19.

For Cas9 nuclease a ddPCR assay was designed capable of simultaneously measuring NHEJ and HDR at endogenous loci. To this end, we designed three kinds of probes, all located within one amplicon. The first, a reference probe, is labeled with FAM and located away from the mutagenesis site. This probe counts all genomic copies of the target. The second, a so-called drop-off probe, is labeled with HEX and is located where the Cas9 nuclease cuts the mlo target. If Cas9 induces NHEJ, the drop-off probe loses its binding site, resulting in loss of HEX and leaving only the FAM signal of the reference probe. The third probe, also FAM-labeled, binds to the desired DNA edit, causing a gain of additional FAM signal when precise edits are introduced. With this assay, indel mutations, WT alleles and precise edits can be detected as distinct, clearly separated droplets with high sensitivity and low background signal.

ddPCR assays were designed against the mlo 5A allele using Primer3Plus software with modified settings compatible with the master mix: that is, 50 mM mono-valent cations, 3.0 mM divalent cations, and 0 mM dNTPs with thermodynamic and salt correction parameters according to SantaLucia (1998). The predicted nuclease cut site (3 bp from the PAM) was positioned mid-amplicon, with 70-100 bp flanking sequence either side up to the primer binding sites. To avoid loss of binding sites, primers and reference probe were designed away from the cut site. In addition, a dark, 3′-phosphorylated non-extendible oligonucleotide was designed to prevent the edit probe from binding to the WT sequence.

PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases.

Considerations for probe design were as follows: probes can bind to either strand of the target, probe GC content of 35-65%, no G at the 5′ end to prevent quenching of the 5′ fluorophore, melting temperature of the drop-off probe ranges from 61° C. to 64° C. with an ideal temperature of 62° C., length of the drop-off probe is less than 20 bases, melting temperatures of the reference and edit probe range from 63° C. to 67° C. with an ideal temperature of 65° C., length of the reference and edit probe of 20-24 bases. Preferably, probes should have a Tm 4-8° C. higher than the primers. Primer and probe designs were also screened for complementarity and secondary structure with the maximum ΔG value of any self-dimers, hairpins, and heterodimers set to −9.0 kcal/mole. All primers and probes were designed against the 5A allele of the wheat mlo gene.

The optimal annealing temperature was empirically determined using a temperature gradient PCR.

Synthetic dsDNA fragments (gBlocks, Integrated DNA Technologies) were used as positive controls for assay validation, HDR-positive controls contain the R158Q substitution at the desired edit site, whereas NHEJ-specific controls have a 1-bp insert at the predicted nuclease cut site. Lyophilized gBlocks were resuspended in 300 μl of TE and stored at min 20° C. Three additional dilutions in TE resulted in a master stock of approximately 600 copies/μl that was confirmed by ddPCR quantification. High-copy gBlock stocks were kept in a post-PCR environment to avoid contamination.

20× ddPCR mixes were composed of 18 μM forward (Seq ID NO: 1) and 18 μM reverse (Seq ID NO: 2) primers, 5 μM reference probe (Seq ID NO: 3), 5 μM edit probe (Seq ID NO: 4), 5 μM drop-off probe (Seq ID NO: 5), and 10 μM dark probe (Seq ID NO: 6). The following reagents were mixed in a 96-well plate to make a 25-μl reaction: 11 μl of ddPCR Supermix for Probes (no dUTP), 1.1 μl of 10× assay mix (BioRad Laboratories), 10U of HindIII-HF, 100-250ng of genomic DNA in water, and water up to 22 μl.

Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories).

Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min.

After PCR, the droplets were analyzed using a QX100 Droplet Reader (Bio-Rad Laboratories) in ‘absolute quantification’ mode. To enable proper gating for precise edits and indel events, experiments were performed using both negative and positive controls (non-modified genomic DNA and gBlocks containing the R158Q mutation, respectively). In two-dimensional plots, droplets without templates were gated as negative population. Droplets containing only NHEJ (FAM+, HEX−), only HDR alleles (FAM++, HEX−) or only WT alleles (FAM+, HEX+) were manually gated as separate populations. Allelic frequencies were quantified using the QuantaSoft v.1.2.10.0 software (BioRad Laboratories).

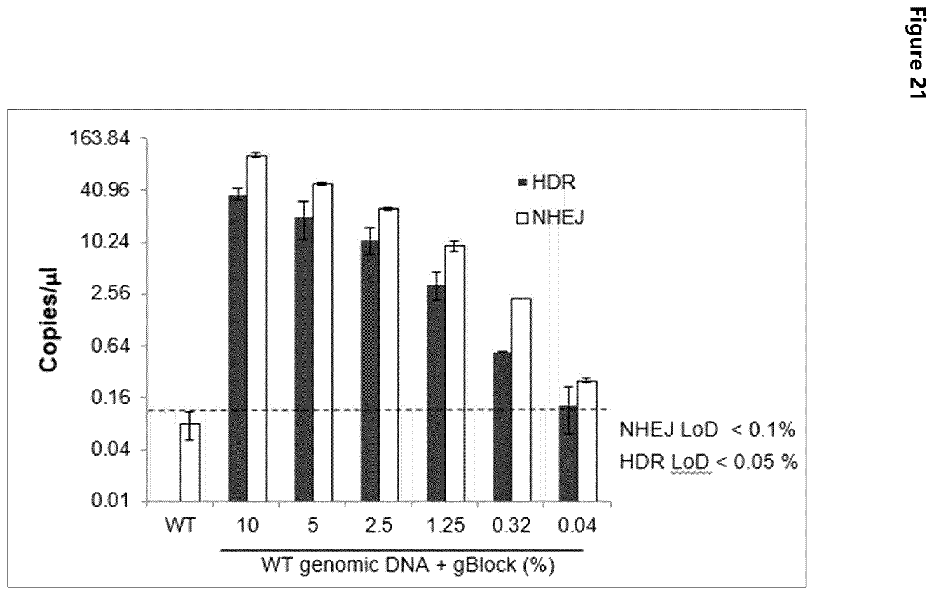

The designed ddPCR assay was verified by next-generation sequencing (NGS) of the target region using a pair of primers specific for the A sub-genome allele of the wheat mlo gene (Seq ID NO: 7/Seq ID NO: 8). The amplicons were purified and subjected to deep-sequencing (2×250 bp paired ends) (GENEWIZ, Germany, GmbH) using an Illumina MiSeq System. A very good correlation (R2=0.96) was observed between the indel allele frequencies detected by ddPCR and NGS across different samples, demonstrating the sensitivity and reliability of the ddPCR assay (FIG. 20).

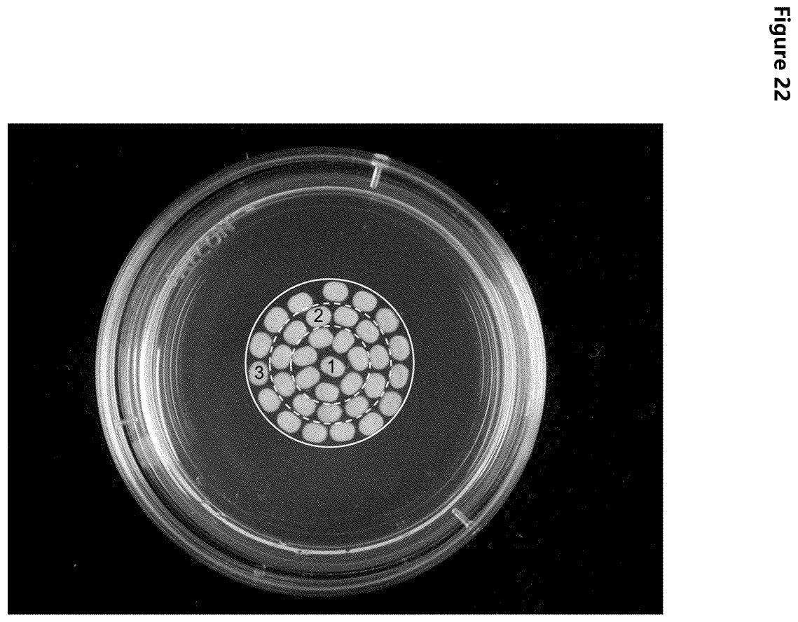

To calculate the ddPCR assay's limit of detection, we spiked wild-type genomic wheat DNA with different amounts of the HDR- and NHEJ-specific gBlocks (Seq ID NO: 9/Seq ID NO: 10) and found that the assay was reproducible and linear over a wide range of input DNA (FIG. 21). The limit of detection was approximately 0.1% for NHEJ. and well below 0.04 for % HDR alleles. This indicates that at least one indel or precise edit event from 1,000 copies of the genome can be captured by the assay.

### Wheat Donor Plant Growth for Immature Embryo Isolation

Donor plants of a highly recalcitrant spring wheat (cv. HR1) are grown under controlled-environment conditions to ensure optimal immature embryo quality. Typical growth conditions are day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds are harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds are washed several times with demineralized water.

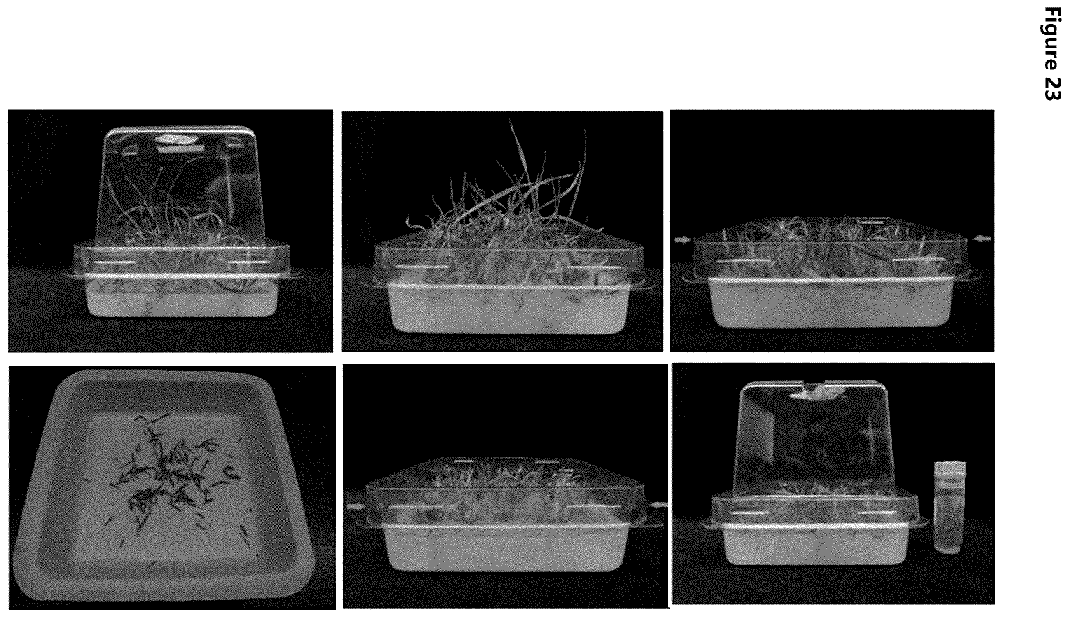

Embryos are aseptically excised from the immature seeds using a binocular microscope (Model MZ6, Leica) taking care to slice-off the embryo axis through the green seed coat during preparation. Subsequent steps for embryo preparation are performed using a modified procedure essentially as described by Ishida et al. (2015). Immature embryo explants are transferred to 3.5 cm Petri dishes (Falcon 351008) containing 4.5 ml of non-selective callus induction medium WLS, herewith designated callus induction medium 240. Embryos are arranged in a central circle of 1.5 cm (25-50 embryos/dish with the scutellum side facing upwards, FIG. 22).

### Preparation of Cas12a RNP Complexes

Purified Cas12a nuclease and mlo-specific crRNA are ordered from IDT (Integrated DNA Technologies) for RNP assembly.
crRNA: custom and user-defined crRNA that binds to 24 bases on the DNA strand that is opposite to the TTTV, PAM sequence.
Cas12a nuclease: (L.b. Cas12a Ultra Nuclease): Recombinant Lachnospiraceae bacterium Cas12a nuclease, purified from E. coli strain expressing Cas12a. Contains C-terminal nuclear localization signal (NLS) and C-terminal 6-His tag.

For RNP complex assembly, mlo-specific crRNA is mixed with Cas12a nuclease in NEBuffer™ 2.1 (New England BioLabs) in an approximate equimolar ratio. The mixture is incubated at 37° C. for 20-30 min and then transferred to ice.

### TaWOX-eGFP-3C-6his Protein

The TaWOX-eGFP-3C-6His coding sequence was synthesized, subcloned into an expression vector and the protein produced and purified from E. coli using the expression platform of ProteoGenix (Schiltigheim, France). The amino acid sequence of TaWOX-eGFP-3C-6His was complemented with a 6 His-tag for purification and a 3C protease site (FIG. 8), although this cleavage site was not used. The TaWOX-eGFP-3C-6His protein was purified in native conditions and thereafter dialyzed and concentrated by ultrafiltration. The TaWOX-eGFP-3C-6His was provided as a 2 μg/μl stock in TBS pH 8 (glycerol 50% v/v).

### Biolistic Co-Delivery of TaWOX-eGFP-3C-6his Protein and Cas12a RNP Complexes

Co-delivery of TaWOX-eGFP-3C-6His protein and Cas12a RNP into immature embryos is performed using the Biolistic® PDS-1000/He Particle Delivery System (Bio-Rad).

For 10 shots, 30 μg of Cas12a protein complexed with crRNA are mixed with 1.65 mg of 0.6 μm gold particles (BioRad) and 24 μl of TransIT-CRISPR® transfection reagent (Sigma-Aldrich) using a modified procedure of Liang et al. (2018). The volume is adjusted to 150 μl with sterile water, incubated for 15 min on ice and centrifuged for 30 sec (10,000 rpm). After removal of the supernatant the pellet is resuspended in a small volume of sterile water, sonicated briefly for few seconds (37 kHz, 50% power in sweep mode, Elmasonic P Sonicator), 40 μg of TaWOX-eGFP-3C-6His protein added and the final volume adjusted to 150 μl. Aliquots of 15 μl are spread over the central region of each macrocarrier, airdried for 60 min in a laminar flow bench and delivered with a pressure of 450 psi with a target distance of approximately 3 cm.

Control bombardments delivering only Cas12a RNP to immature embryos are performed as described above except the TaWOX-eGFP-3C-6His protein is omitted.

Following bombardment, the immature embryos are incubated on the same plates for 48 h in the dark (280C+/−1° C., 55% relative humidity in an MLR-352H-PE Panasonic incubator).

### Culture of Bombarded Wheat Immature Embryos and Plant Regeneration

After 48 h the embryos are transferred to fresh non-selective callus induction medium 240 (9 cm dishes containing 35-40 ml of medium with up to 15 embryos/dish) and incubated in the dark (25° C.+/−1° C.).

Seven days after bombardment immature embryos are longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

After 2 weeks, the bisected immature embryos are once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo are transferred intact to fresh non-selective callus induction medium 240 (9 cm dishes containing 35-40 ml of medium, 2 embryos/dish) and cultured in the dark under the same conditions.

Two weeks later, small pieces of structured embryogenic callus tissue are transferred to a non-selective regeneration medium, LSZ (Ishida et al., 2015), herewith designated regeneration medium 420. Regeneration medium 420 is prepared in PlantCon™™ containers (MP Biomedicals, Catalogue Nr. 26-722-06), 100 ml medium/container. The embryogenic calli arising from one or more embryo(s) are transferred to a PlantCon™ container (max. 16 calli/container). PlantCon™ containers are incubated in the light for approximately 6 weeks (23° C.+/−1° C., 16 h photoperiod).

Shoots from PlantCon™ containers are transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod).

### Tissue Samplinq and DNA Isolation

Sampling 1—Three days after bombardment, 5 immature embryos are randomly selected from each bombarded plate. The embryos are bulked as one sample, collected in 2 ml tubes (Eppendorf® Safe-Lock) and stored at −80°. Samples are ground in a Retsch Mixer Mill MM300/400 for 60 sec. Genomic DNA extraction is performed using the Qiagen Dneasy Plant Mini Kit (Catalogue Nr. 69106) according to the manufacturer's instructions. Final DNA concentrations are measured, and plates stored at 4° C. until use for downstream analysis.

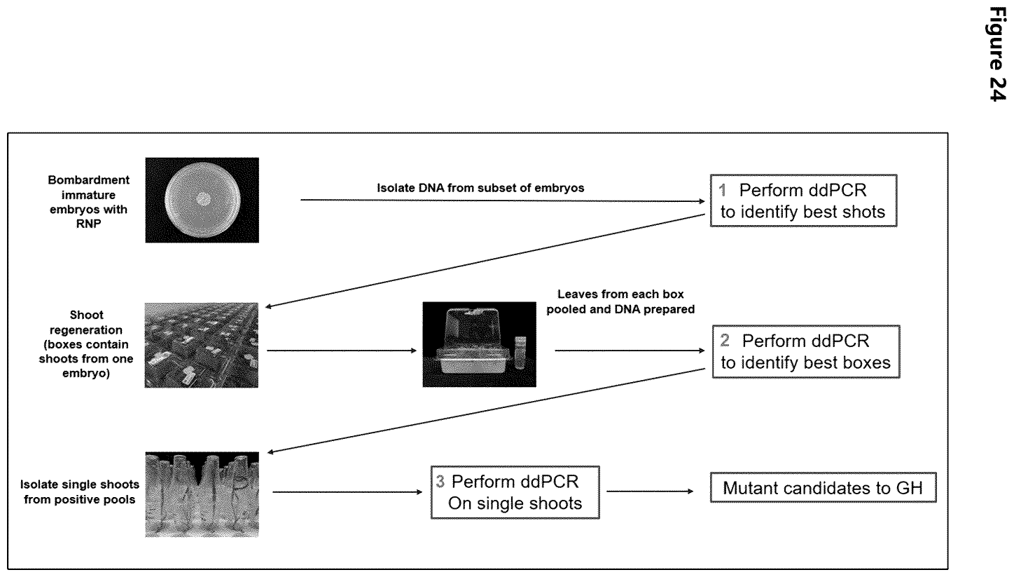

Sampling 2—Following 4 weeks of culture of structured embryogenic callus on non-selective regeneration medium 420 in PlantCon™ containers, leaf pieces are harvested from regenerating shoots. Care is taken to ensure that similar sized leaf pieces are taken from all regenerating shoots, this is best achieved by first cutting off all longer leaf tips with sterile scissors and then cutting leaf pieces of approximately 5 mm in length from the remaining tissues (FIG. 23). Leaf pieces originating from each PlantCon™ container are pooled and transferred to 6 ml screw-capped tubes (Micronic MP 32301) and stored at −80°. Samples are freeze dried overnight and genomic DNA extracted using a procedure based on the LGC GENOMICS Sbeadex™ Maxi Plant Kit with KingFisher automation. Final DNA concentrations are measured, and plates stored at 4° C. until use for downstream analysis.

Sampling 3—Following 1-2 weeks of culture of individual shoots in De Wit tubes containing 10 ml of WRM medium, leaf pieces are harvested from shoots. Samples are collected in 1.4 ml push-cap tubes in a 96-sample carrier rack (Micronic MP 226RP) and stored at −80°. Genomic DNA is extracted using a procedure based on the LGC GENOMICS Sbeadex™ Maxi Plant Kit with KingFisher automation. Final DNA concentrations are measured, and plates stored at 4° C. until use for downstream analysis.

### Molecular Selection to Identify Cas12a RNP Indels in Mlo

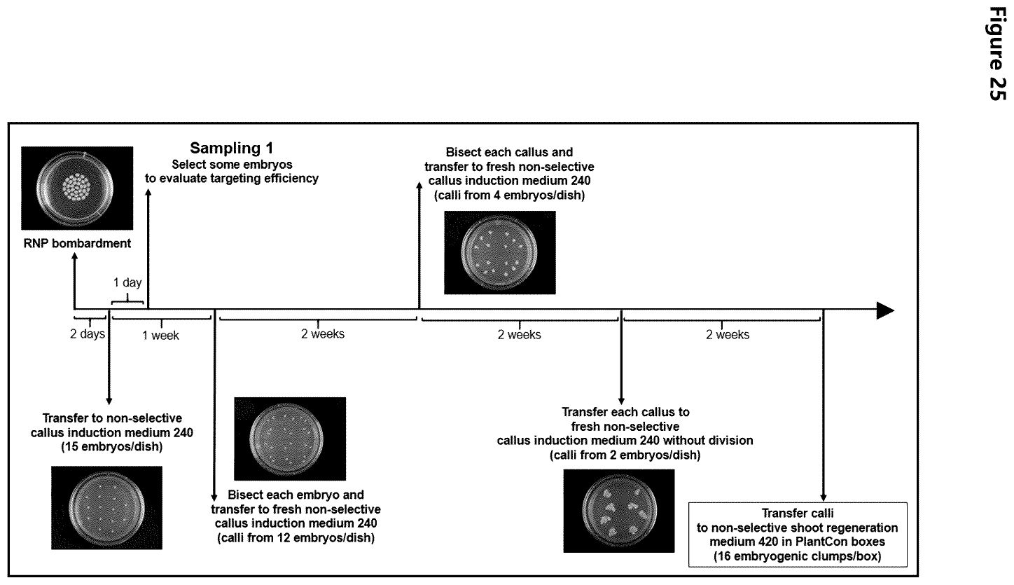

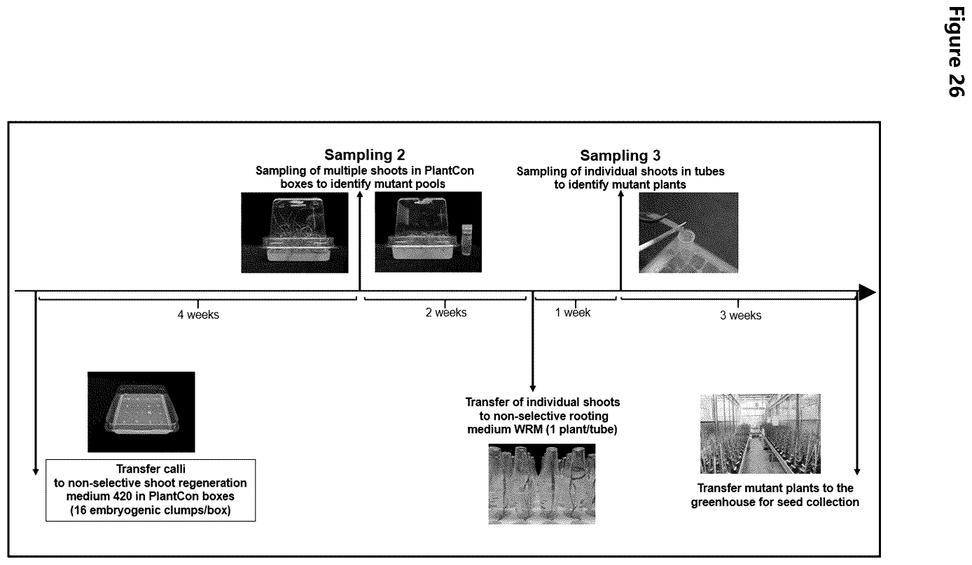

An overview of the 3-step selection system to identify RNP-induced indels in mlo in wheat plants is shown in FIG. 24. The process involves sequential enrichment for tissues carrying RNP-induced mlo INDELS through repeated sampling and molecular screening to specifically detect targeted mutations. This is performed in 3 steps, in immature embryo explants, in regenerated populations of shoots and finally at the single plant level. A detailed overview of the selection/analysis process for Cas12a RNP is shown in FIG. 25 and FIG. 26.

Three days after biolistic co-delivery of Cas12a RNP and TaWOX-eGFP-3C-6His protein, 5 immature embryos from each shot are selected and sampled for DNA isolation and ddPCR analysis (ddPCR droplet generator Biorad+ PCR machine Sensoquest+ ddPCR reader Biorad) to determine mlo NHEJ drop-off percentages. 20× ddPCR mixes are composed of 18 μM forward (Seq ID NO: 1) and 18 μM reverse (Seq ID NO: 2) primers, 5 μM reference probe (Seq ID NO: 3) and 5 μM drop-off probe (Seq ID NO: 5). The following reagents are mixed in a 96-well plate to make a 25-μl reaction: 11 μl of ddPCR Supermix for Probes (no dUTP), 1.1 μl of 10× assay mix (BioRad Laboratories), 100-250ng of genomic DNA in water, and water up to 22 μl.

The results of the ddPCR analysis are visualized as two-dimensional plots generated by Quantasoft Software to provide an estimate of drop-off percentages (i.e. the percentage of amplicons carrying indels in mlo relative to the percentage of amplicons representing WT). Immature embryos are selected from bombardments showing the highest drop-off percentages for further culture as these ones are most likely to later generate shoots with NHEJ indels in mlo alleles.

Selected immature embryos are passed-through 3 cycles of culture on non-selective callus induction medium 240 to obtain structured embryogenic callus from which shoots can be regenerated on non-selective regeneration medium 420 in PlantCon™ containers. Leaf pieces originating from multiple shoots in each PlantCon™ container are pooled and transferred to 6 ml screw-capped tubes (Micronic MP 32301) for ddPCR analysis to again determine mlo drop-off percentages. Here, differences in regeneration potential, between embryos bombarded with Cas12a RNP and Cas12a RNP+ TaWOX-eGFP-3C-6His protein are observed at the plant level and also in ddPCR drop-off analysis. The total number of regenerating shoots in containers derived from embryos bombarded with Cas12a RNP+ TaWOX-eGFP-3C-6His protein is visually higher than from embryos bombarded with only Cas12a RNP. At the ddPCR analysis level, differences in drop-off percentages are also observed. Higher mlo drop-off values are seen for pools of shoots derived from embryos bombarded with Cas12a RNP+ TaWOX-eGFP-3C-6His protein, as more of the RNP-targeted cells have the potential to regenerate as they have also received TaWOX-eGFP-3C-6His protein. In summary, TaWOX-eGFP-3C-6His protein is beneficial for improving the total yield of regenerated plants from a certain population of embryos and consequently also for increasing the number of Cas12a mlo targeted alleles.

Shoots from pools showing highest ddPCR drop-off percentages are selected for further culture as these pools are the ones most likely to generate plants with NHEJ indels in mlo alleles. Shoots from selected PlantCon™ containers are transferred to individual De Wit tubes containing 10 ml of non-selective rooting medium WRM for further development. Approximately 2 weeks after transfer, leaf samples from individual plants are taken for ddPCR analysis to determine mlo drop-off percentages. Shoots are assigned to specific classes based on the drop-off percentages obtained with the ddPCR assay designed for the A sub-genome (i.e. values close to 0%=WT, values close to 50%=monoallelic mutation in mlo and values close to 100%=biallelic mutation in mlo).

Individual plants are selected for NGS to confirm the ddPCR results of the mlo A subgenomic copy. 500 ng of purified PCR products (primer combination Seq ID NO: 11/Seq ID NO: 12) are sent for NGS (GENEWIZ® Germany GmbH). The Amplicon-EZ Illumina-based service provides full sequence coverage of PCR products up to 500 bp in length (up to 50.000 reads/sample are delivered). The raw Illumina data is analyzed and visualized using the software application CLC Genomics Workbench 12.0.3 (Qiagen) and proprietary CRISPRMapper plugin.

### Example 8 Co-Delivery of TaWox mRNA and CRISPR Cas12a RNP for Enhanced Recovery of Mlo Mutants in Wheat

qRNA Design for Cas12a Targeting of Mlo

Building upon previous work (Gil-Humanes et al. 2017) we designed a synthetic crRNA composed of a 21 bp direct repeat sequence and a 24 bp protospacer targeting the wheat mildew resistance locus O (mlo). The mlo gene encodes a seven-transmembrane domain protein involved in resistance to the fungal pathogen Blumeria graminis. The recognition site of the crRNA is located on the antisense strand within exon 4 of mlo [5′-[(TTTG)CGAACTGGTATTCCAAGGAGGCGG-3′], with PAM site between brackets. The designed guide is specific for the 5A and 4D alleles of mlo and shows one mismatch with the 4B allele at position 16 from the PAM sequence (FIG. 18).

Design of a Droplet Digital PCR (ddPCR) Assay to Detect Cas12a RNP-Induced Mlo Indel Mutations

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

The ddPCR assay used to identify Cas12a-RNP induced mlo indels was the same as that previously designed for the detection of Cas9-RNP induced mlo indels (as described below). The location of the predicted Cas12a cut site in the mlo gene and the position of the PCR primers and probes used for the ddPCR analysis are shown in FIG. 19.

For Cas9 nuclease a ddPCR assay was designed capable of simultaneously measuring NHEJ and HDR at endogenous loci. To this end, we designed three kinds of probes, all located within one amplicon. The first, a reference probe, is labeled with FAM and located away from the mutagenesis site. This probe counts all genomic copies of the target. The second, a so-called drop-off probe, is labeled with HEX and is located where the Cas9 nuclease cuts the mlo target. If Cas9 induces NHEJ, the drop-off probe loses its binding site, resulting in loss of HEX and leaving only the FAM signal of the reference probe. The third probe, also FAM-labeled, binds to the desired DNA edit, causing a gain of additional FAM signal when precise edits are introduced. With this assay, indel mutations, WT alleles and precise edits can be detected as distinct, clearly separated droplets with high sensitivity and low background signal.

ddPCR assays were designed against the mlo 5A allele using Primer3Plus software with modified settings compatible with the master mix: that is, 50 mM mono-valent cations, 3.0 mM divalent cations, and 0 mM dNTPs with thermodynamic and salt correction parameters according to SantaLucia (1998). The predicted nuclease cut site (3 bp from the PAM) was positioned mid-amplicon, with 70-100 bp flanking sequence either side up to the primer binding sites. To avoid loss of binding sites, primers and reference probe were designed away from the cut site. In addition, a dark, 3′-phosphorylated non-extendible oligonucleotide was designed to prevent the edit probe from binding to the WT sequence.

PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases.

Considerations for probe design were as follows: probes can bind to either strand of the target, probe GC content of 35-65%, no G at the 5′ end to prevent quenching of the 5′ fluorophore, melting temperature of the drop-off probe ranges from 61° C. to 64° C. with an ideal temperature of 62° C., length of the drop-off probe is less than 20 bases, melting temperatures of the reference and edit probe range from 63° C. to 67° C. with an ideal temperature of 65° C., length of the reference and edit probe of 20-24 bases. Preferably, probes should have a Tm 4-8° C. higher than the primers. Primer and probe designs were also screened for complementarity and secondary structure with the maximum ΔG value of any self-dimers, hairpins, and heterodimers set to −9.0 kcal/mole. All primers and probes were designed against the 5A allele of the wheat mlo gene.

The optimal annealing temperature was empirically determined using a temperature gradient PCR.

Synthetic dsDNA fragments (gBlocks, Integrated DNA Technologies) were used as positive controls for assay validation, HDR-positive controls contain the R158Q substitution at the desired edit site, whereas NHEJ-specific controls have a 1-bp insert at the predicted nuclease cut site. Lyophilized gBlocks were resuspended in 300 μl of TE and stored at min 20° C. Three additional dilutions in TE resulted in a master stock of approximately 600 copies/μl that was confirmed by ddPCR quantification. High-copy gBlock stocks were kept in a post-PCR environment to avoid contamination.

20× ddPCR mixes were composed of 18 μM forward (Seq ID NO: 1) and 18 μM reverse (Seq ID NO: 2) primers, 5 μM reference probe (Seq ID NO: 3), 5 μM edit probe (Seq ID NO: 4), 5 μM drop-off probe (Seq ID NO: 5), and 10 μM dark probe (Seq ID NO: 6). The following reagents were mixed in a 96-well plate to make a 25-μl reaction: 11 μl of ddPCR Supermix for Probes (no dUTP), 1.1 μl of 10× assay mix (BioRad Laboratories), 10U of HindIII-HF, 100-250ng of genomic DNA in water, and water up to 22 μl.

Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories).

Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min.

After PCR, the droplets were analyzed using a QX100 Droplet Reader (Bio-Rad Laboratories) in ‘absolute quantification’ mode. To enable proper gating for precise edits and indel events, experiments were performed using both negative and positive controls (non-modified genomic DNA and gBlocks containing the R158Q mutation, respectively). In two-dimensional plots, droplets without templates were gated as negative population. Droplets containing only NHEJ (FAM+, HEX−), only HDR alleles (FAM++, HEX−) or only WT alleles (FAM+, HEX+) were manually gated as separate populations. Allelic frequencies were quantified using the QuantaSoft v.1.2.10.0 software (BioRad Laboratories).

The designed ddPCR assay was verified by next-generation sequencing (NGS) of the target region using a pair of primers specific for the A sub-genome allele of the wheat mlo gene (Seq ID NO: 7/Seq ID NO: 8). The amplicons were purified and subjected to deep-sequencing (2×250 bp paired ends) (GENEWIZ, Germany, GmbH) using an Illumina MiSeq System. A very good correlation (R2=0.96) was observed between the indel allele frequencies detected by ddPCR and NGS across different samples, demonstrating the sensitivity and reliability of the ddPCR assay (FIG. 20).

To calculate the ddPCR assay's limit of detection, we spiked wild-type genomic wheat DNA with different amounts of the HDR- and NHEJ-specific gBlocks (Seq ID NO: 9/Seq ID NO: 10) and found that the assay was reproducible and linear over a wide range of input DNA (FIG. 21). The limit of detection was approximately 0.1% for NHEJ. and well below 0.04 for % HDR alleles. This indicates that at least one indel or precise edit event from 1,000 copies of the genome can be captured by the assay.

### Wheat Donor Plant Growth for Immature Embryo Isolation

Donor plants of a highly recalcitrant spring wheat (cv. HR1) are grown under controlled-environment conditions to ensure optimal immature embryo quality. Typical growth conditions are day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds are harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds are washed several times with demineralized water.

Embryos are aseptically excised from the immature seeds using a binocular microscope (Model MZ6, Leica) taking care to slice-off the embryo axis through the green seed coat during preparation. Subsequent steps for embryo preparation are performed using a modified procedure essentially as described by Ishida et al. (2015). Immature embryo explants are transferred to 3.5 cm Petri dishes (Falcon 351008) containing 4.5 ml of non-selective callus induction medium WLS, herewith designated callus induction medium 240. Embryos are arranged in a central circle of 1.5 cm (25-50 embryos/dish with the scutellum side facing upwards).

### Preparation of Cas12a RNP Complexes

Purified Cas12a nuclease and mlo-specific crRNA are ordered from IDT (Integrated DNA Technologies) for RNP assembly.
crRNA: custom and user-defined crRNA that binds to 24 bases on the DNA strand that is opposite to the TTTV, PAM sequence.
Cas12a nuclease: (L.b. Cas12a Ultra Nuclease): Recombinant Lachnospiraceae bacterium Cas12a nuclease, purified from E. coli strain expressing Cas12a. Contains C-terminal nuclear localization signal (NLS) and C-terminal 6-His tag.

For RNP complex assembly, mlo-specific crRNA is mixed with Cas12a nuclease in NEBuffer™ 2.1 (New England BioLabs) in an approximate equimolar ratio. The mixture is incubated at 37° C. for 20-30 min and then transferred to ice.

### TaWox-mRNA

The TaWox gene coding sequence (from vector Pubi-wox5-3.1.2), flanked by 5′ and 3′ UTR sequences originating from the maize Ubiquitine 1 gene (Christensen et al., 1992) is synthetically made as a gBlock ds DNA fragment (IDT-DNA) and cloned into a pUC57-Kan cloning vector using standard molecular cloning techniques to generate vector P-Wox5-mRNA (FIG. 17). The resulting vector was then used with primers (Seq ID NO: 30/Seq ID NO: 31), to produce a PCR product serving as a template for RNA in vitro transcription. A T7 promoter was included in the 5′ end of the forward PCR primer (Seq ID NO: 30). TaWox mRNA is in vitro transcribed using a HiScribe T7 ARCA mRNA Kit (with tailing) cat. No NEB #E2060S (New England Biolabs) according to manufacturer's instructions. A DNAse step is included for the removal of the DNA template. The resulting ARCA capped (5′) and poly(A) tailed egfp mRNA is purified using a Monarch RNA Cleanup Kit Cat. no NEB #T2050S/L (New England Biolabs) and eluted into 50 μl of nuclease-free water (end concentration approximately 1 μg/μl). The mRNA integrity is confirmed before and after tailing using an Agilent 2100 Bioanalyzer with an RNA 6000 Nano assay chip according to manufacturer's instructions.

Biolistic Co-Delivery of TaWox mRNA and Cas12a RNP Complexes

Co-delivery of TaWox mRNA and Cas12a RNP into immature embryos is performed using the Biolistic® PDS-1000/He Particle Delivery System (Bio-Rad).

For 10 shots, 30 μg of Cas12a protein complexed with crRNA, and 10 μg TaWox mRNA are mixed with 1.65 mg of 0.6 μm gold particles (BioRad) and 24 μl of TransIT-CRISPR® transfection reagent (Sigma-Aldrich) using a modified procedure of Liang et al. (2018). The volume is adjusted to 150 μl with sterile water, incubated for 5 min on ice and centrifuged for 10 sec (12,000 rpm). After removal of the supernatant the pellet is resuspended in 150 μl of sterile water, sonicated briefly (37 kHz, 50% power in sweep mode, Elmasonic P Sonicator. Aliquots of 15 μl are spread over the central region of each macrocarrier, airdried for 60 min in a laminar flow bench and delivered with a pressure of 450 psi with a target distance of approximately 3 cm.

Control bombardments delivering only Cas12a RNP to immature embryos are performed as described above except the TaWox mRNA is omitted.

Following bombardment, the immature embryos are incubated on the same plates for 48 h in the dark (28° C.+/−1° C., 55% relative humidity in an MLR-352H-PE Panasonic incubator).

### Culture of Bombarded Wheat Immature Embryos and Plant Regeneration

After 48 h the embryos are transferred to fresh non-selective callus induction medium 240 (9 cm dishes containing 35-40 ml of medium with up to 15 embryos/dish) and incubated in the dark (25° C.+/−1° C.).

Seven days after bombardment immature embryos are longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

After 2 weeks, the bisected immature embryos are once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo are transferred intact to fresh non-selective callus induction medium 240 (9 cm dishes containing 35-40 ml of medium, 2 embryos/dish) and cultured in the dark under the same conditions.

Two weeks later, small pieces of structured embryogenic callus tissue are transferred to a non-selective regeneration medium, LSZ (Ishida et al., 2015), herewith designated regeneration medium 420. Regeneration medium 420 is prepared in PlantCon™™ containers (MP Biomedicals, Catalogue Nr. 26-722-06), 100 ml medium/container. The embryogenic calli arising from one or more embryo(s) are transferred to a PlantCon™ container (max. 16 calli/container). PlantCon™ containers are incubated in the light for approximately 6 weeks (23° C.+/−1° C., 16 h photoperiod).

Shoots from PlantCon™ containers are transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod).

### Tissue Samplinq and DNA Isolation

Sampling 1—Three days after bombardment, 5 immature embryos are randomly selected from each bombarded plate. The embryos are bulked as one sample, collected in 2 ml tubes (Eppendorf® Safe-Lock) and stored at −80°. Samples are ground in a Retsch Mixer Mill MM300/400 for 60 sec. Genomic DNA extraction is performed using the Qiagen Dneasy Plant Mini Kit (Catalogue Nr. 69106) according to the manufacturer's instructions. Final DNA concentrations are measured, and plates stored at 4° C. until use for downstream analysis.

Sampling 2—Following 4 weeks of culture of structured embryogenic callus on non-selective regeneration medium 420 in PlantCon™ containers, leaf pieces are harvested from regenerating shoots. Care is taken to ensure that similar sized leaf pieces are taken from all regenerating shoots, this is best achieved by first cutting off all longer leaf tips with sterile scissors and then cutting leaf pieces of approximately 5 mm in length from the remaining tissues (as in FIG. 23). Leaf pieces originating from each PlantCon™ container are pooled and transferred to 6 ml screw-capped tubes (Micronic MP 32301) and stored at −80°. Samples are freeze dried overnight and genomic DNA extracted using a procedure based on the LGC GENOMICS Sbeadex™ Maxi Plant Kit with KingFisher automation. Final DNA concentrations are measured, and plates stored at 4° C. until use for downstream analysis.

Sampling 3—Following 1-2 weeks of culture of individual shoots in De Wit tubes containing 10 ml of WRM medium, leaf pieces are harvested from shoots. Samples are collected in 1.4 ml push-cap tubes in a 96-sample carrier rack (Micronic MP 226RP) and stored at −80°. Genomic DNA is extracted using a procedure based on the LGC GENOMICS Sbeadex™ Maxi Plant Kit with KingFisher automation. Final DNA concentrations are measured, and plates stored at 4° C. until use for downstream analysis.

### Molecular Selection to Identify Cas12a RNP Indels in Mlo

An overview of the 3-step selection system to identify RNP-induced indels in mlo in wheat plants is shown in FIG. 24. The process involves sequential enrichment for tissues carrying RNP-induced mlo INDELS through repeated sampling and molecular screening to specifically detect targeted mutations. This is performed in 3 steps, in immature embryo explants, in regenerated populations of shoots and finally at the single plant level. A detailed overview of the selection/analysis process for Cas12a RNP is shown in FIG. 25 and FIG. 26.

Three days after biolistic co-delivery of Cas12a RNP and TaWox mRNA, 5 immature embryos from each shot are selected and sampled for DNA isolation and ddPCR analysis (ddPCR droplet generator Biorad+PCR machine Sensoquest+ ddPCR reader Biorad) to determine mlo NHEJ drop-off percentages. 20× ddPCR mixes are composed of 18 μM forward (Seq ID NO: 1) and 18 μM reverse (Seq ID NO: 2) primers, 5 μM reference probe (Seq ID NO: 3) and 5 μM drop-off probe (Seq ID NO: 5). The following reagents are mixed in a 96-well plate to make a 25-μl reaction: 11 μl of ddPCR Supermix for Probes (no dUTP), 1.1 μl of 10× assay mix (BioRad Laboratories), 100-250ng of genomic DNA in water, and water up to 22 μl.

The results of the ddPCR analysis are visualized as two-dimensional plots generated by Quantasoft Software to provide an estimate of drop-off percentages (i.e. the percentage of amplicons carrying indels in mlo relative to the percentage of amplicons representing WT). Immature embryos are selected from bombardments showing the highest drop-off percentages for further culture as these ones are most likely to later generate shoots with NHEJ indels in mlo alleles.

Selected immature embryos are passed-through 3 cycles of culture on non-selective callus induction medium 240 to obtain structured embryogenic callus from which shoots can be regenerated on non-selective regeneration medium 420 in PlantCon™ containers. Leaf pieces originating from multiple shoots in each PlantCon™ container are pooled and transferred to 6 ml screw-capped tubes (Micronic MP 32301) for ddPCR analysis to again determine mlo drop-off percentages. Here, differences in regeneration potential, between embryos bombarded with Cas12a RNP and Cas12a RNP+ TaWox mRNA are observed at the plant level and also in ddPCR drop-off analysis. The total number of regenerating shoots in containers derived from embryos bombarded with Cas12a RNP+ TaWox mRNA is visually higher than from embryos bombarded with only Cas12a RNP. At the ddPCR analysis level, differences in drop-off percentages are also observed. Higher mlo drop-off values are seen for pools of shoots derived from embryos bombarded with Cas12a RNP+ TaWox mRNA, as more of the RNP-targeted cells have the potential to regenerate as they have also received TaWox mRNA. In summary, TaWox mRNA is beneficial for improving the total yield of regenerated plants from a certain population of embryos and consequently also for increasing the number of Cas12a mlo targeted alleles.

Shoots from pools showing highest ddPCR drop-off percentages are selected for further culture as these pools are the ones most likely to generate plants with NHEJ indels in mlo alleles. Shoots from selected PlantCon™ containers are transferred to individual De Wit tubes containing 10 ml of non-selective rooting medium WRM for further development. Approximately 2 weeks after transfer, leaf samples from individual plants are taken for ddPCR analysis to determine mlo drop-off percentages. Shoots are assigned to specific classes based on the drop-off percentages obtained with the ddPCR assay designed for the A sub-genome (i.e. values close to 0%=WT, values close to 50%=monoallelic mutation in mlo and values close to 100%=biallelic mutation in mlo).

Individual plants are selected for NGS to confirm the ddPCR results of the mlo A subgenomic copy. 500 ng of purified PCR products (primer combination Seq ID NO: 11/Seq ID NO: 12) are sent for NGS (GENEWIZ® Germany GmbH). The Amplicon-EZ Illumina-based service provides full sequence coverage of PCR products up to 500 bp in length (up to 50.000 reads/sample are delivered). The raw Illumina data is analyzed and visualized using the software application CLC Genomics Workbench 12.0.3 (Qiagen) and proprietary CRISPRMapper plugin.

### Example 9: Co-Delivery of TaWOX-eGFP-3C-6his Protein, TaWox mRNA and CRISPR Cas12a RNP for Enhanced Recovery of Mlo Mutants in Wheat

qRNA Design for Cas12a Targeting of Mlo

Building upon previous work (Gil-Humanes et al. 2017) we designed a synthetic crRNA composed of a 21 bp direct repeat sequence and a 24 bp protospacer targeting the wheat mildew resistance locus O (mlo). The mlo gene encodes a seven-transmembrane domain protein involved in resistance to the fungal pathogen Blumeria graminis. The recognition site of the crRNA is located on the antisense strand within exon 4 of mlo [5′-[(TTTG)CGAACTGGTATTCCAAGGAGGCGG-3′], with PAM site between brackets (SEQ ID NO: 34). The designed guide is specific for the 5A and 4D alleles of mlo and shows one mismatch with the 4B allele at position 16 from the PAM sequence (FIG. 18).

Design of a Droplet Digital PCR (ddPCR) Assay to Detect Cas12a RNP-Induced Mlo Indel Mutations

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

The ddPCR assay used to identify Cas12a-RNP induced mlo indels was the same as that previously designed for the detection of Cas9-RNP induced mlo indels (as described below). The location of the predicted Cas12a cut site in the mlo gene and the position of the PCR primers and probes used for the ddPCR analysis are shown in FIG. 19.

For Cas9 nuclease a ddPCR assay was designed capable of simultaneously measuring NHEJ and HDR at endogenous loci. To this end, we designed three kinds of probes, all located within one amplicon. The first, a reference probe, is labeled with FAM and located away from the mutagenesis site. This probe counts all genomic copies of the target. The second, a so-called drop-off probe, is labeled with HEX and is located where the Cas9 nuclease cuts the mlo target. If Cas9 induces NHEJ, the drop-off probe loses its binding site, resulting in loss of HEX and leaving only the FAM signal of the reference probe. The third probe, also FAM-labeled, binds to the desired DNA edit, causing a gain of additional FAM signal when precise edits are introduced. With this assay, indel mutations, WT alleles and precise edits can be detected as distinct, clearly separated droplets with high sensitivity and low background signal.

ddPCR assays were designed against the mlo 5A allele using Primer3Plus software with modified settings compatible with the master mix: that is, 50 mM mono-valent cations, 3.0 mM divalent cations, and 0 mM dNTPs with thermodynamic and salt correction parameters according to SantaLucia (1998). The predicted nuclease cut site (3 bp from the PAM) was positioned mid-amplicon, with 70-100 bp flanking sequence either side up to the primer binding sites. To avoid loss of binding sites, primers and reference probe were designed away from the cut site. In addition, a dark, 3′-phosphorylated non-extendible oligonucleotide was designed to prevent the edit probe from binding to the WT sequence.

PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases.

Considerations for probe design were as follows: probes can bind to either strand of the target, probe GC content of 35-65%, no G at the 5′ end to prevent quenching of the 5′ fluorophore, melting temperature of the drop-off probe ranges from 61° C. to 64° C. with an ideal temperature of 62° C., length of the drop-off probe is less than 20 bases, melting temperatures of the reference and edit probe range from 63° C. to 67° C. with an ideal temperature of 65° C., length of the reference and edit probe of 20-24 bases. Preferably, probes should have a Tm 4-8° C. higher than the primers. Primer and probe designs were also screened for complementarity and secondary structure with the maximum ΔG value of any self-dimers, hairpins, and heterodimers set to −9.0 kcal/mole. All primers and probes were designed against the 5A allele of the wheat mlo gene.

The optimal annealing temperature was empirically determined using a temperature gradient PCR.

Synthetic dsDNA fragments (gBlocks, Integrated DNA Technologies) were used as positive controls for assay validation, HDR-positive controls contain the R158Q substitution at the desired edit site, whereas NHEJ-specific controls have a 1-bp insert at the predicted nuclease cut site. Lyophilized gBlocks were resuspended in 300 μl of TE and stored at min 20° C. Three additional dilutions in TE resulted in a master stock of approximately 600 copies/μl that was confirmed by ddPCR quantification. High-copy gBlock stocks were kept in a post-PCR environment to avoid contamination. [0295]20× ddPCR mixes were composed of 18 μM forward (Seq ID NO: 1) and 18 μM reverse (Seq ID NO: 2) primers, 5 μM reference probe (Seq ID NO: 3), 5 μM edit probe (Seq ID NO: 4), 5 μM drop-off probe (Seq ID NO: 5), and 10 μM dark probe (Seq ID NO: 6). The following reagents were mixed in a 96-well plate to make a 25-μl reaction: 11 μl of ddPCR Supermix for Probes (no dUTP), 1.1 μl of 10× assay mix (BioRad Laboratories), 10U of HindIII-HF, 100-250ng of genomic DNA in water, and water up to 22 μl.

Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories).

Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min.

After PCR, the droplets were analyzed using a QX100 Droplet Reader (Bio-Rad Laboratories) in ‘absolute quantification’ mode. To enable proper gating for precise edits and indel events, experiments were performed using both negative and positive controls (non-modified genomic DNA and gBlocks containing the R158Q mutation, respectively). In two-dimensional plots, droplets without templates were gated as negative population. Droplets containing only NHEJ (FAM+, HEX−), only HDR alleles (FAM++, HEX−) or only WT alleles (FAM+, HEX+) were manually gated as separate populations. Allelic frequencies were quantified using the QuantaSoft v.1.2.10.0 software (BioRad Laboratories).

The designed ddPCR assay was verified by next-generation sequencing (NGS) of the target region using a pair of primers specific for the A sub-genome allele of the wheat mlo gene (Seq ID NO: 7/Seq ID NO: 8). The amplicons were purified and subjected to deep-sequencing (2×250 bp paired ends) (GENEWIZ, Germany, GmbH) using an Illumina MiSeq System. A very good correlation (R2=0.96) was observed between the indel allele frequencies detected by ddPCR and NGS across different samples, demonstrating the sensitivity and reliability of the ddPCR assay (FIG. 20).

To calculate the ddPCR assay's limit of detection, we spiked wild-type genomic wheat DNA with different amounts of the HDR- and NHEJ-specific gBlocks (Seq ID NO: 9/Seq ID NO: 10) and found that the assay was reproducible and linear over a wide range of input DNA (FIG. 21). The limit of detection was approximately 0.1% for NHEJ. and well below 0.04 for % HDR alleles. This indicates that at least one indel or precise edit event from 1,000 copies of the genome can be captured by the assay.

### Wheat Donor Plant Growth for Immature Embryo Isolation

Donor plants of a highly recalcitrant spring wheat (cv. HR1) are grown under controlled-environment conditions to ensure optimal immature embryo quality. Typical growth conditions are day/night temperatures of 20° C.+/−1° C. and 18° C.+/−1° C., 65% relative humidity with a 16 h photoperiod (400 μmol/m²/s illumination at table level) provided by a mixture of 600 W high pressure sodium lamps and 400 W metal halide lamps.

### Preparation of Wheat Immature Embryos for Bombardment

Immature seeds are harvested from donor plants containing embryos around 2 mm in length, peeled and sterilized in 70% v/v ethanol for 1 min and followed by 10 min in 10% v/v sodium hypochlorite (ACROS bleach containing 10-15% active chlorine). Finally, the immature seeds are washed several times with demineralized water.

Embryos are aseptically excised from the immature seeds using a binocular microscope (Model MZ6, Leica) taking care to slice-off the embryo axis through the green seed coat during preparation. Subsequent steps for embryo preparation are performed using a modified procedure essentially as described by Ishida et al. (2015). Immature embryo explants are transferred to 3.5 cm Petri dishes (Falcon 351008) containing 4.5 ml of non-selective callus induction medium WLS, herewith designated callus induction medium 240. Embryos are arranged in a central circle of 1.5 cm (25-50 embryos/dish with the scutellum side facing upwards, FIG. 22).

### Preparation of Cas12a RNP Complexes

Purified Cas12a nuclease and mlo-specific crRNA are ordered from IDT (Integrated DNA Technologies) for RNP assembly.
crRNA: custom and user-defined crRNA that binds to 24 bases on the DNA strand that is opposite to the TTTV, PAM sequence.
Cas12a nuclease: (L.b. Cas12a Ultra Nuclease): Recombinant Lachnospiraceae bacterium Cas12a nuclease, purified from E. coli strain expressing Cas12a. Contains C-terminal nuclear localization signal (NLS) and C-terminal 6-His tag.

For RNP complex assembly, mlo-specific crRNA is mixed with Cas12a nuclease in NEBuffer™ 2.1 (New England BioLabs) in an approximate equimolar ratio. The mixture is incubated at 37° C. for 20-30 min and then transferred to ice.

### TaWOX-eGFP-3C-6his Protein

The TaWOX-eGFP-3C-6His coding sequence was synthesized, subcloned into an expression vector and the protein produced and purified from E. coli using the expression platform of ProteoGenix (Schiltigheim, France). The amino acid sequence of TaWOX-eGFP-3C-6His was complemented with a 6 His-tag for purification and a 3C protease site (FIG. 8), although this cleavage site was not used. The TaWOX-eGFP-3C-6His protein was purified in native conditions and thereafter dialyzed and concentrated by ultrafiltration. The TaWOX-eGFP-3C-6His was provided as a 2 μg/μl stock in TBS pH 8 (glycerol 50% v/v).

### TaWox-mRNA

The TaWox gene coding sequence (from vector Pubi-wox5-3.1.2), flanked by 5′ and 3′ UTR sequences originating from the maize Ubiquitine 1 gene (Christensen et al., 1992) is synthetically made as a gBlock ds DNA fragment (IDT-DNA) and cloned into a pUC57-Kan cloning vector using standard molecular cloning techniques to generate vector P-Wox5-mRNA (FIG. 17). The resulting vector was then used with primers (Seq ID NO: 30/Seq ID NO: 31), to produce a PCR product serving as a template for RNA in vitro transcription. A T7 promoter was included in the 5′ end of the forward PCR primer (Seq ID NO: 30). TaWox mRNA is in vitro transcribed using a HiScribe T7 ARCA mRNA Kit (with tailing) cat. No NEB #E2060S (New England Biolabs) according to manufacturer's instructions. A DNAse step is included for the removal of the DNA template. The resulting ARCA capped (5′) and poly(A) tailed egfp mRNA is purified using a Monarch RNA Cleanup Kit Cat. no NEB #T2050S/L (New England Biolabs) and eluted into 50 μl of nuclease-free water (end concentration approximately 1 μg/μl). The mRNA integrity is confirmed before and after tailing using an Agilent 2100 Bioanalyzer with an RNA 6000 Nano assay chip according to manufacturer's instructions.

Biolistic Co-Delivery of TaWOX-eGFP-3C-6his Protein, TaWox mRNA and Cas12a RNP Complexes

Co-delivery of TaWOX-eGFP-3C-6His protein, TaWox mRNA and Cas12a RNP into immature embryos is performed using the Biolistic® PDS-1000/He Particle Delivery System (Bio-Rad).

For 10 shots, 30 μg of Cas12a protein complexed with crRNA, and 10 μg TaWox mRNA are mixed with 1.65 mg of 0.6 μm gold particles (BioRad) and 24 μl of TransIT-CRISPR® transfection reagent (Sigma-Aldrich) using a modified procedure of Liang et al. (2018). The volume is adjusted to 150 μl with sterile water, incubated for 5 min on ice and centrifuged for 10 sec (12,000 rpm). After removal of the supernatant the pellet is resuspended in a small volume of sterile water, sonicated briefly for few seconds (37 kHz, 50% power in sweep mode, Elmasonic P Sonicator), 40 μg of TaWOX-eGFP-3C-6His protein added and the final volume adjusted to 150 μl. Aliquots of 15 μl are spread over the central region of each macrocarrier, airdried for 60 min in a laminar flow bench and delivered with a pressure of 450 psi with a target distance of approximately 3 cm.

Control bombardments delivering only Cas12a RNP to immature embryos are performed as described above except the TaWOX-eGFP-3C-6His protein and TaWox mRNA are omitted.

Following bombardment, the immature embryos are incubated on the same plates for 48 h in the dark (280C+/−1° C., 55% relative humidity in an MLR-352H-PE Panasonic incubator).

### Culture of Bombarded Wheat Immature Embryos and Plant Regeneration

After 48 h the embryos are transferred to fresh non-selective callus induction medium 240 (9 cm dishes containing 35-40 ml of medium with up to 15 embryos/dish) and incubated in the dark (25° C.+/−1° C.).

Seven days after bombardment immature embryos are longitudinal bisected into 2 pieces under a binocular microscope and transferred to fresh non-selective callus induction medium 240 (9 cm dishes containing 35-40 ml of medium, 12 embryos/dish) and cultured in the dark (25° C.+/−1° C.).

After 2 weeks, the bisected immature embryos are once more bisected under a binocular microscope and transferred to fresh non-selective callus induction medium 240 (9 cm dishes containing 35-40 ml of medium, 4-6 embryos/dish) and cultured in the dark under the same conditions. After a further 2 weeks, the calli from each embryo are transferred intact to fresh non-selective callus induction medium 240 (9 cm dishes containing 35-40 ml of medium, 2 embryos/dish) and cultured in the dark under the same conditions.

Two weeks later, small pieces of structured embryogenic callus tissue are transferred to a non-selective regeneration medium, LSZ (Ishida et al., 2015), herewith designated regeneration medium 420. Regeneration medium 420 is prepared in PlantCon™™ containers (MP Biomedicals, Catalogue Nr. 26-722-06), 100 ml medium/container. The embryogenic calli arising from one or more embryo(s) are transferred to a PlantCon™ container (max. 16 calli/container). PlantCon™ containers are incubated in the light for approximately 6 weeks (23° C.+/−1° C., 16 h photoperiod).

Shoots from PlantCon™ containers are transferred individually to De Wit tubes (Duchefa Biochemie) containing 10 ml of non-selective rooting medium WRM, essentially a modification of medium R (Sparks and Jones, 2009), solidified with 0.15% w/v Gelrite (Duchefa Biochemie) and cultured in the light (23° C.+/−1° C., 16 h photoperiod).

### Tissue Samplinq and DNA Isolation

Sampling 1 —Three days after bombardment, 5 immature embryos are randomly selected from each bombarded plate. The embryos are bulked as one sample, collected in 2 ml tubes (Eppendorf® Safe-Lock) and stored at −80°. Samples are ground in a Retsch Mixer Mill MM300/400 for 60 sec. Genomic DNA extraction is performed using the Qiagen Dneasy Plant Mini Kit (Catalogue Nr. 69106) according to the manufacturer's instructions. Final DNA concentrations are measured, and plates stored at 4° C. until use for downstream analysis.

Sampling 2—Following 4 weeks of culture of structured embryogenic callus on non-selective regeneration medium 420 in PlantCon™ containers, leaf pieces are harvested from regenerating shoots. Care is taken to ensure that similar sized leaf pieces are taken from all regenerating shoots, this is best achieved by first cutting off all longer leaf tips with sterile scissors and then cutting leaf pieces of approximately 5 mm in length from the remaining tissues (FIG. 23). Leaf pieces originating from each PlantCon™ container are pooled and transferred to 6 ml screw-capped tubes (Micronic MP 32301) and stored at −80°. Samples are freeze dried overnight and genomic DNA extracted using a procedure based on the LGC GENOMICS Sbeadex™ Maxi Plant Kit with KingFisher automation. Final DNA concentrations are measured, and plates stored at 4° C. until use for downstream analysis.

Sampling 3—Following 1-2 weeks of culture of individual shoots in De Wit tubes containing 10 ml of WRM medium, leaf pieces are harvested from shoots. Samples are collected in 1.4 ml push-cap tubes in a 96-sample carrier rack (Micronic MP 226RP) and stored at −80°. Genomic DNA is extracted using a procedure based on the LGC GENOMICS Sbeadex™ Maxi Plant Kit with KingFisher automation. Final DNA concentrations are measured, and plates stored at 4° C. until use for downstream analysis.

### Molecular Selection to Identify Cas12a RNP Indels in mlo

An overview of the 3-step selection system to identify RNP-induced indels in mlo in wheat plants is shown in FIG. 24. The process involves sequential enrichment for tissues carrying RNP-induced mlo INDELS through repeated sampling and molecular screening to specifically detect targeted mutations. This is performed in 3 steps, in immature embryo explants, in regenerated populations of shoots and finally at the single plant level. A detailed overview of the selection/analysis process for Cas12a RNP is shown in FIG. 25 and FIG. 26.

Three days after biolistic co-delivery of Cas12a RNP+ TaWOX-eGFP-3C-6His protein+ TaWox mRNA, 5 immature embryos from each shot are selected and sampled for DNA isolation and ddPCR analysis (ddPCR droplet generator Biorad+PCR machine Sensoquest+ ddPCR reader Biorad) to determine mlo NHEJ drop-off percentages. 20× ddPCR mixes are composed of 18 μM forward (Seq ID NO: 1) and 18 μM reverse (Seq ID NO: 2) primers, 5 μM reference probe (Seq ID NO: 3) and 5 μM drop-off probe (Seq ID NO: 5). The following reagents are mixed in a 96-well plate to make a 25-μl reaction: 11 μl of ddPCR Supermix for Probes (no dUTP), 1.1 μl of 10× assay mix (BioRad Laboratories), 100-250ng of genomic DNA in water, and water up to 22 μl.

The results of the ddPCR analysis are visualized as two-dimensional plots generated by Quantasoft Software to provide an estimate of drop-off percentages (i.e. the percentage of amplicons carrying indels in mlo relative to the percentage of amplicons representing WT). Immature embryos are selected from bombardments showing the highest drop-off percentages for further culture as these ones are most likely to later generate shoots with NHEJ indels in mlo alleles.

Selected immature embryos are passed-through 3 cycles of culture on non-selective callus induction medium 240 to obtain structured embryogenic callus from which shoots can be regenerated on non-selective regeneration medium 420 in PlantCon™ containers. Leaf pieces originating from multiple shoots in each PlantCon™ container are pooled and transferred to 6 ml screw-capped tubes (Micronic MP 32301) for ddPCR analysis to again determine mlo drop-off percentages. Here, differences in regeneration potential, between embryos bombarded with Cas12a RNP and Cas12a RNP+ TaWOX-eGFP-3C-6His protein+TaWox mRNA are observed at the plant level and also in ddPCR drop-off analysis. The total number of regenerating shoots in containers derived from embryos bombarded with Cas12a RNP+TaWOX-eGFP-3C-6His e3protein+TaWox mRNA is visually higher than from embryos bombarded with only Cas12a RNP. At the ddPCR analysis level, differences in drop-off percentages are also observed. Higher mlo drop-off values are seen for pools of shoots derived from embryos bombarded with Cas12a RNP+ TaWOX-eGFP-3C-6His protein+TaWox mRNA, as more of the RNP-targeted cells have the potential to regenerate as they have also received TaWOX-eGFP-3C-6His protein and TaWox mRNA. In summary, the combination of TaWOX-eGFP-3C-6His protein and TaWox mRNA is more beneficial than delivering either TaWOX-eGFP-3C-6His protein or TaWox mRNA alone, for improving the total yield of regenerated plants from a certain population of embryos and consequently also for increasing the number of Cas12a mlo targeted alleles.

Shoots from pools showing highest ddPCR drop-off percentages are selected for further culture as these pools are the ones most likely to generate plants with NHEJ indels in mlo alleles. Shoots from selected PlantCon™ containers are transferred to individual De Wit tubes containing 10 ml of non-selective rooting medium WRM for further development. Approximately 2 weeks after transfer, leaf samples from individual plants are taken for ddPCR analysis to determine mlo drop-off percentages. Shoots are assigned to specific classes based on the drop-off percentages obtained with the ddPCR assay designed for the A sub-genome (i.e. values close to 0%=WT, values close to 50%=monoallelic mutation in mlo and values close to 100%=biallelic mutation in mlo).

Individual plants are selected for NGS to confirm the ddPCR results of the mlo A subgenomic copy. 500 ng of purified PCR products (primer combination Seq ID NO: 11/Seq ID NO: 12) are sent for NGS (GENEWIZ® Germany GmbH). The Amplicon-EZ Illumina-based service provides full sequence coverage of PCR products up to 500 bp in length (up to 50.000 reads/sample are delivered). The raw Illumina data is analyzed and visualized using the software application CLC Genomics Workbench 12.0.3 (Qiagen) and proprietary CRISPRMapper plugin.

### Example 10: Identification of Sequences Related to SEQ ID NO: 13 and SEQ ID NO: 14

Sequences (full length cDNA, ESTs or genomic) related to SEQ ID NO: 13 and SEQ ID NO: 14 can be identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 13 can be used for the TBLASTN algorithm, with default settings and the filter to ignore low complexity sequences set off. The output of the analysis is then viewed by pairwise comparison, and ranked according to the probability score (E-value), where the score reflect the probability that a particular alignment occurs by chance (the lower the E-value, the more significant the hit). In addition to E-values, comparisons can also be scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Sequences have been tentatively assembled and publicly disclosed by research institutions, such as The Institute for Genomic Research (TIGR; beginning with TA). For instance, the Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, e.g. for certain prokaryotic organisms, such as by the Joint Genome Institute. Furthermore, access to proprietary databases, has allowed the identification of novel nucleic acid and polypeptide sequences.

Table A provides a list of sequences of TaWOX homologues that are useful in the methods of the present invention.

TABLE A

| | Nucleic acid | Protein |
|---|---|---|
| Plant Source | SEQ ID NO: | SEQ ID NO: |
| Triticum aestivum | SEQ ID NO: 13 | SEQ ID NO: 14 |
| Triticum aestivum | SEQ ID NO: 17 | SEQ ID NO: 18 |
| Triticum aestivum | SEQ ID NO: 19 | SEQ ID NO: 20 |
| Oryza sativa | SEQ ID NO: 21 | SEQ ID NO: 22 |
| Zea mays | SEQ ID NO: 23 | SEQ ID NO: 24 |
| Zea mays | SEQ ID NO: 25 | SEQ ID NO: 26 |
| Arabidopsis thaliana | SEQ ID NO: 27 | SEQ ID NO: 28 |

### REFERENCES

Christensen A H, Sharrock R A, Quail P H. Maize polyubiquitin genes: structure, thermal perturbation of expression and transcript splicing, and promoter activity following transfer to protoplasts by electroporation. Plant Mol Biol 1992, February 18(4):675-689.
Cormack, B P, Valdivia, R H, Falkow, S. A versatile vector system for generating recombinant eGFP-tagged proteins in yeast. Gene 1996, 173:33-38.
Depicker A, Stachel S, Dhaese P, Zambryski P, Goodman H M. Nopaline synthase: transcript mapping and DNA sequence. J Mol Appl Genet 1982, 1(6):561-73.
Gil-Humanes J, Wang Y, Liang Z, Shan Q, Ozuna C V, Sánchez-León S, Baltes N J, Starker C, Barro F, Gao C, Voytas D F. High-efficiency gene targeting in hexaploid wheat using DNA replicons and CRISPR/Cas9. Plant J 2017, March; 89(6):1251-1262.
Harpster M H, Townsend J A, Jones J D, Bedbrook J, Dunsmuir P. Relative strengths of the 35S cauliflower mosaic virus, 1′,2′, and nopaline synthase promoters in transformed tobacco sugar beet and oilseed rape callus tissue. Mol Gen Genet 1988, April; 212(1):182-190.
Ishida Y, Tsunashima M, Hiei Y, Komari T. Wheat (Triticum aestivum L.) transformation using immature embryos. Methods Mol Biol 2015; 1223:189-98.
Jefferson R A, Kavanagh T A, Bevan M W. GUS fusions: β-glucuronidase as a sensitive and versatile gene fusion marker in higher plants. EMBO J 1987, 6(13):3901-3907.
Liang Z, Chen K, Zhang Y, Liu J, Yin K, Qiu J L, Gao C. Genome editing of bread wheat using biolistic delivery of CRISPR/Cas9 in vitro transcripts or ribonucleoproteins. Nat Protoc 2018, March; 13(3):413-430.
Odell J T, Nagy F, Chua N H. Identification of DNA sequences required for activity of the cauliflower mosaic virus 35S promoter. Nature 1985, 6;313(6005):810-812.
Sanfaç on, H, Brodmann, Hohn, P T. A dissection of the cauliflower mosaic virus polyadenylation signal. Genes Dev 1991, January; 5(1):141-149.
SantaLucia, J. A unified view of polymer, dumbbell, and oligonucleotide DNA nearest-neighbor thermodynamics. Proc. Natl Acad. Sci. USA 1998, 95 1460-1465.
Sparks C A, Jones H D. Biolistics transformation of wheat. In: Jones H D, Shewry P R, editors. Transgenic wheat, barley and oats: production and characterization protocols.
Totowa: Humana Press; 2009. p. 71-92.
Sparks C A and Jones H D. Genetic transformation of wheat via particle bombardment.
Cereal Genomics: Methods and Protocols, Methods in Molecular Biology; 2014, vol. 1099, p. 201-218.
Thompson C J, Movva N R, Tizzard R, Crameri, R, Davies J E, Lauwereys M, Botterman J. Characterization of the herbicide-resistance gene bar from Streptomyces hygroscopicus. EMBO J 1987, September; 6(9):2519-2523.
Zhao S, Jiang Q-T, Ma J, Zhang X-W, Zhao Q-Z, Wang X-Y, Wang C-S, Cao X, Lu Z-X, Zheng Y-L, Wei Y-M. Characterization and expression analysis of WOX5 genes from wheat and its relatives. Gene 2014, March; 537(1):63-69.

1. A method for increasing efficiency of regenerating a genome edited plant cell into a plant, comprising the steps of:
(a) simultaneously introducing into a plant cell (i) an endonuclease designed for a desired genome modification (EDTGM) and (ii) a TaWOX, wherein said endonuclease and said TaWOX are introduced as protein or as its encoding mRNA;
(b) allowing the EDTGM to modify the genome of said plant cell; and
(c) regenerating said plant cell into a plant, thereby allowing the introduced TaWOX to increase efficiency of regeneration;
wherein the regeneration efficiency is increased compared to the regeneration of corresponding genome-edited plant cells without having said TaWOX introduced.

2. The method of claim 1, wherein said TaWOX is a transcription factor comprising a Wuschel-like InterPro family IPR044555 domain.

3. The method of claim 2, wherein the TaWOX further comprises a WUS-box motif and an ERF-associated amphiphilic repression motif.

4. The method of claim 1, wherein said TaWOX is a polypeptide comprising a sequence having at least 70%, 75%, 80%, 85%, 90%, 95%, 97% 97.2%, 97.5% 98%, 98.5%, 99%, 99.5%, 99.7%, 99.8%, 99.9% or more amino acid sequence identity to the TaWOX polypeptide represented by SEQ ID NO: 14.

5. The method of claim 1, wherein said TaWOX is a polypeptide comprising the sequence represented by SEQ ID NO: 14.

6. The method of claim 1, wherein said TaWOX is a polypeptide with the sequence represented by SEQ ID NO: 14.

7. The method according to claim 1, wherein said TaWOX protein is fused to a reporter protein.

8. The method of claim 1, wherein said TaWOX mRNA encodes a transcription factor comprising a Wuschel-like InterPro family IPR044555 domain.

9. The method of claim 2, wherein said TaWOX is a protein substantially similar to the endogenous orthologue of SEQ ID NO: 14, naturally present in said genome edited plant cell.

10. The method of claim 1, wherein said EDTGM is selected from: a CRISPR-Cas system, a Zn-finger endonuclease, a TALEN endonuclease, or a meganuclease.

11. The method of claim 1, wherein said endonuclease is a Class 2 CRISPR-Cas type endonuclease.

12. The method of claim 1, wherein said simultaneous introduction into a cell is obtained by one of particle bombardment, electroporation, cationic lipid vesicles, nanoparticles, aerosol beam microinjection or sonoporation.

13. The method of claim 1, wherein said increased regeneration efficiency is a higher percentage of cells regenerating into a plant compared to genome-edited plant cells without having said TaWOX introduced.

14. The method of claim 1, wherein said plant cell is derived from a plant recalcitrant to regeneration.

15. A method for producing a genome edited plant comprising the steps of
(a) simultaneously introducing into a plant cell (i) an EDTGM and (ii) a TaWOX, wherein said EDTGM and said TaWOX are introduced as protein or as its encoding mRNA;
(b) allowing the EDTGM to modify the genome of said plant cell; and
(c) regenerating said plant cell into a plant, thereby allowing the introduced TaWOX to increase efficiency of regeneration;
wherein the regeneration efficiency is increased compared to the regeneration of corresponding genome edited plant cells without having said TaWOX introduced.

16. A method for increasing efficiency of genome editing of a plant cell comprising
a) simultaneously introducing into a plant cell (i) an EDTGM and (ii) a TaWOX, wherein said endonuclease and said TaWOX are introduced as protein or as its encoding mRNA;
b) allowing the EDTGM to modify the genome of said plant cell; and
c) regenerating said plant cell into a plant, thereby allowing the introduced TaWOX to increase efficiency of regeneration;
wherein the regeneration efficiency is increased compared to the regeneration of corresponding genome-edited plant cells without having said TaWOX introduced.

17. The method of claim 16, wherein said regenerated plant has improved agronomic and/or nutritional properties.

18. The method of claim 7, wherein said TaWOX protein is fused to a screenable marker protein.

19. The method of claim 18, wherein said screenable marker is a fluorescent marker, preferably a GFP

20. The method of claim 18, wherein the fusion protein is represented by SEQ ID NO: 16.

21. A particle designed for biolistic delivery into a cell, coated with a mixture of (i) an EDTGM and a TaWOX fused to a reporter, in the form of mRNA or protein; or (ii) an EDTGM, a TaWOX and a reporter in the form of mRNA or protein.

22. (canceled)

23. A plant cell comprising a particle according to claim 21.

24. A method for increasing efficiency of regeneration of a genome edited plant cell into a plant or for increasing the efficiency of genome editing of a plant cell comprising using the particle of claim 21.

25. (canceled)


SEQUENCE LISTING (40 sequences)

SEQ ID NO: 1 - DNA, 17 bp, synthetic construct
  source 1..17: mol_type = other DNA
cctgctccggcataata

SEQ ID NO: 2 - DNA, 17 bp, synthetic construct
  source 1..17: mol_type = other DNA
tcagttcactcgctctg

SEQ ID NO: 3 - DNA, 23 bp, synthetic construct
  source 1..23: mol_type = other DNA
tttgatgcgtttacagaggcagg

SEQ ID NO: 4 - DNA, 23 bp, synthetic construct
  source 1..23: mol_type = other DNA
tttcttccatgtctgcatctgcc

SEQ ID NO: 5 - DNA, 19 bp, synthetic construct
  source 1..19: mol_type = other DNA
agaccgcctccttggaata

SEQ ID NO: 6 - DNA, 23 bp, synthetic construct
  source 1..23: mol_type = other DNA
tttcttccatgttctcatctgcc

SEQ ID NO: 7 - DNA, 23 bp, synthetic construct
  source 1..23: mol_type = other DNA
caaggtggcgctcatcgtacgtc

SEQ ID NO: 8 - DNA, 23 bp, synthetic construct
  source 1..23: mol_type = other DNA
cgctctgatcatgcctgcctctg

SEQ ID NO: 9 - DNA, 401 bp, synthetic construct
  source 1..401: mol_type = other DNA
gtggttgttggctgtggcggcaggttcccagtcgaaaaggacgccaaagaggaagtaaagctcttcttgggtaatgctggaatcgcaatgcggagcctgacggcagctgtagtagctgctggtggaaatgcaacgtatgttttctttatcctagtggaaataagtatgagatccatgggtatgtttggagactgatcgtttcttttattaaaaaaaaacttcagttatgtgcttgatggcgtaccaagaatgagggagcgacctattggtgacttagttgtaggtttgcaacaactcggcgcagatgtcgattgtttccttggcacaaactgcccacctgtccgtatcaacggcaaaggaggtctacctggtggcaaggttagctactcatcaacttgcat

SEQ ID NO: 10 - DNA, 392 bp, synthetic construct
  source 1..392: mol_type = other DNA
gtgagcctttctttctttctttcccgtgcttccagatcctgcgcggttcccgggcaaggtggcgctcatcgtacgtctgtctcagttaaactgctaccaatccttaacctgctccggcataatattcttattcctccccccggcagatgaaaacatggaagaaatgggagacagagaccgcctaccttggaataccagttcgcaaatggtcagacaattttccaaatgaaacctcttctgttttgatgcgtttacagaggcaggcatgatcagagcgagtgaactgatgatatgttcttctctttcccgtgcttccagatcctgcgcggttccgcttcacgcaccagacgtcgttcgtgaagcggcacctgggcctgtccagcacccccggc

SEQ ID NO: 11 - DNA, 21 bp, synthetic construct
  source 1..21: mol_type = other DNA
atcatggctctaagccgtctc

SEQ ID NO: 12 - DNA, 20 bp, synthetic construct
  source 1..20: mol_type = other DNA
tctggtgcgtgaagcggaac

SEQ ID NO: 13 - DNA, 633 bp, Triticum sp.
  source 1..633: mol_type = other DNA
atggaggctctctccggccgcgtcggcgtcaagtgtggaagatggaaccccaccgccgagcaagtcaaggtgctcaccgagctcttcagagctggcttgaggacaccatcaactgagcagatccagaggatctccactcatctctcagccttcggcaaggtggagagcaagaatgtcttctactggttccagaaccacaaggcaagagaaaggcatcatcacaagaagagaagaagggtggctagttgttctcctgacagctcaagcaacgacgaggagactggaagggccgccgccgccgagccagcagatctagttcttcaacctccagagagcaagagagaagcaagaggctacaaccatcatccaaggatcatgacatgctatgttcgagaggtggcagagcaagaagaagccaccacctgggagaggccaacaagagaggtggaaacccttgagctcttccccctcaaggccgcctgctatgatctggagctggaggctgacaggttctcaaggtacgtcagatctggagagcagcaatgtcgagagatctccttctttgatgtggccaccggccgagatcctcctctggagctgaggctctgctccttcgacaggtacctggtgtag

SEQ ID NO: 14 - protein, 210 aa, Triticum sp.
MEALSGRVGVKCGRWNPTAEQVKVLTELFRAGLRTPSTEQIQRISTHLSAFGKVESKNVFYWFQNHKARERHHHKKRRRVASCSPDSSSNDEETGRAAAAEPADLVLQPPESKREARGYNHHPRIMTCYVREVAEQEEATTWERPTREVETLELFPLKAACYDLELEADRFSRYVRSGEQQCREISFFDVATGRDPPLELRLCSFDRYLV

SEQ ID NO: 15 - DNA, 1424 bp, synthetic construct
  source 1..1424: mol_type = other DNA
ccatggaagcactgagcggtcgtgttggtgtgaaatgcggtcgttggaatccgaccgcagaacaggtgaaagtgctgaccgaactgtttcgcgcaggcctgcgcaccccgagtaccgaacagattcagcgcattagtacccatctgagtgcctttggtaaagttgaaagcaaaaatgttttctactggtttcagaatcacaaagcccgtgaacgtcatcatcataaaaaacgtcgtcgcgttgccagttgtagcccggatagcagtagtaatgatgaagaaaccggccgtgccgcagcagccgaaccggcagatctggtgctgcagccgccggaaagtaaacgtgaagcacgtggctataatcatcatccgcgtattatgacctgctatgttcgtgaagttgccgaacaggaagaagcaaccacctgggaacgtccgacccgtgaagtggaaaccctggaactgtttccgctgaaagcagcatgttatgatctggaactggaagccgatcgctttagtcgttatgtgcgtagcggtgaacagcagtgccgcgaaattagtttctttgatgttgcaaccggccgtgatccgccgctggaactgcgcctgtgcagctttgatcgctatctggttggcggtggtagcggtggtggcgttagcaaaggcgaagaactgtttaccggcgttgttccgattctggttgaactggatggtgacgttaatggtcataaattttcagtgagtggcgaaggtgaaggtgacgcaacctatggtaaactgaccctgaaattcatttgtaccaccggcaaactgccggtgccgtggccgaccctggttaccaccctgacctatggtgtgcagtgttttagccgttatccggatcatatgaaacagcatgatttctttaagagcgccatgccggaaggttatgtgcaggaacgcaccattttctttaaagatgatggtaattacaagacccgcgcagaagttaaatttgaaggcgataccctggttaatcgcattgaactgaaaggcattgattttaaagaggatggtaatattctgggtcataaactggaatataactataatagccacaacgtttacatcatggccgataaacagaaaaatggtattaaggttaacttcaagatccgtcataatattgaagatggtagtgtgcagctggcagatcattatcagcagaataccccgattggcgatggcccggtgctgctgccggataatcattatctgagcacccagagtgccctgagcaaagatccgaatgaaaaacgcgatcatatggttctgctggaatttgtgaccgccgcaggtattaccctgggtatggatgaactgtataaactggaagtgctgtttcagggcccgggtagccatcatcatcatcaccattaactcgag

SEQ ID NO: 16 - protein, 471 aa, synthetic construct
MEALSGRVGVKCGRWNPTAEQVKVLTELFRAGLRTPSTEQIQRISTHLSAFGKVESKNVFYWFQNHKARERHHHKKRRRVASCSPDSSSNDEETGRAAAAEPADLVLQPPESKREARGYNHHPRIMTCYVREVAEQEEATTWERPTREVETLELFPLKAACYDLELEADRFSRYVRSGEQQCREISFFDVATGRDPPLELRLCSFDRYLVGGGSGGGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEVLFQGPGSHHHHHH

SEQ ID NO: 17 - DNA, 639 bp, Triticum aestivum
  source 1..639: mol_type = other DNA
atggaggctctctccggccgcgtcggcgtcaagtgtggaagatggaaccccaccgccgagcaagtcaaggtgctcaccgagctcttcagagctggcttgaggacaccatcaactgagcagatccagaggatctccactcatctctcagccttcggcaaggtggagagcaagaatgtcttctactggttccagaaccacaaggcaagagaaaggcatcatcacaagaagagaagaagggtggctagttgttctcctgacagctcaagcaacgaggaggagactggaagggccgccggccgagatgcagaaccagcagatctagttcttcaacctccagagagcaagagagaagcaagaggctacaaccatcatccaaggatcatgacatgctatgttcgtgaggtggccgagcaagaagaagccaccacctgggagaggccaacaagagaggtggagacactggagctcttccccctcaaggccgcctgctatgatcttgagctggaggctgacaggttctcaaggtatgtccgaggaggagagcagcaatgtcgagagatctccttctttgatgtggccaccggccgagatcctcctctggagctgaggctctgctccttcgacaggtacctggtgtag

SEQ ID NO: 18 - protein, 212 aa, Triticum aestivum
MEALSGRVGVKCGRWNPTAEQVKVLTELFRAGLRTPSTEQIQRISTHLSAFGKVESKNVFYWFQNHKARERHHHKKRRRVASCSPDSSSNEEETGRAAGRDAEPADLVLQPPESKREARGYNHHPRIMTCYVREVAEQEEATTWERPTREVETLELFPLKAACYDLELEADRFSRYVRGGEQQCREISFFDVATGRDPPLELRLCSFDRYLV

SEQ ID NO: 19 - DNA, 630 bp, Triticum aestivum
  source 1..630: mol_type = other DNA
atggaggctctctccggccgcgtcggcgtcaagtgtggaagatggaaccccaccgccgagcaagtcaaggtgctcaccgagctcttcagagctggcttgaggacaccatcaactgagcagatccaaaggatctccactcatctctcagccttcggcaaggtggagagcaagaatgtcttctactggttccagaaccacaaggcaagagaaaggcatcatcacaagaagagaagaagggtggctagttgttctcctgacagctcaagcaacgaggaggagtccggcagggccgccgccgccgagccagcagatctagttctccagccaccagaaagcaagagagaagcaagaggctacaaccatcatccaaggatcatgacatgctatgttcgagaagtcaccgagcaagaagaagccaccacctgggagaggccaacaagagaggtggaaacccttgagctcttccccctcaaggccgcctatgatctggagctggaggctgacaggttctcaaggtatgtgagaggaggagagcagcaatgtcgagagatctccttctttgatgtggccaccggccgagatcctcctctggagcttcgtctctgctccttcgacaggtacctggtgtaa

SEQ ID NO: 20 - protein, 209 aa, Triticum aestivum
MEALSGRVGVKCGRWNPTAEQVKVLTELFRAGLRTPSTEQIQRISTHLSAFGKVESKNVFYWFQNHKARERHHHKKRRRVASCSPDSSSNEEESGRAAAAEPADLVLQPPESKREARGYNHHPRIMTCYVREVTEQEEATTWERPTREVETLELFPLKAAYDLELEADRFSRYVRGGEQQCREISFFDVATGRDPPLELRLCSFDRYLV

SEQ ID NO: 21 - DNA, 603 bp, Oryza sativa
  source 1..603: mol_type = other DNA
atggaggctctctccggccgcgtcggcgtcaagtgtggaagatggaaccccaccgccgagcaagtcaaggtgctcaccgagctcttcagagctggcttgaggacaccatcaacagagcagatccagaggatctccacccatctctcagccttcggcaaggtggagagcaagaatgtcttctactggttccagaaccacaaggctagagaacggcatcatcacaagaagaggaggagaggagcttcatcaccagattccggcagcaatgatgatgatggaagggcagcagctcatgaaggagatgcagatctagttctccagccaccagagagcaagagagaagcaagaagctatggccatcatcatcgcctcatgacatgctatgttcgagatgtggtggagactgaggccatgtgggagaggccaacaagagaggtggagacactggagctcttccccctcaagtcatatgatcttgaggtggacaaggtgaggtatgtaagaggaggaggaggagaacaatgtcgagagatctccttcttcgatgtggccgccggccgagatcctcctctggagctgaggctctgctccttcggcctttag

SEQ ID NO: 22 - protein, 200 aa, Oryza sativa
MEALSGRVGVKCGRWNPTAEQVKVLTELFRAGLRTPSTEQIQRISTHLSAFGKVESKNVFYWFQNHKARERHHHKKRRRGASSPDSGSNDDDGRAAAHEGDADLVLQPPESKREARSYGHHHRLMTCYVRDVVETEAMWERPTREVETLELFPLKSYDLEVDKVRYVRGGGGEQCREISFFDVAAGRDPPLELRLCSFGL

SEQ ID NO: 23 - DNA, 666 bp, Zea mays
  source 1..666: mol_type = other DNA
atggaggccttgtcaggccgcgtcggcgtcaagtgtggaagatggaaccccaccgccgagcaagtcaaggtgctcaccgagctcttcagagctggcttgaggacaccatccaccgagcagatccaaaggataagcacccatctctcagccttcggcaaggtggagagcaagaatgtcttctactggttccagaaccacaaggctcgtgaaaggcatcatcacaagaagaggaggagaggagcttcatcaagctcaccagattcaggatctggaagaggatctaacaacgaggaagatggccgaggagcagcaagccaaagccatgatgctgatgccgacgccgatctagtccttcaacctccagagagcaagagagaagcaagaagctatggccatcatcatcgcctcgtcacctgctatgttcgagatgtggtggagcagcaagaagcatcaccatcatgggagaggccaacaagagaggtggagacactggagctcttccctctcaagagctatggagatcttgaggccgccgagaaggtgagaagctatgtaagaggaagcggcgccacctcagagcaatgtcgagagctctccttcttcgatgtggtgtccgccggcagagatcctcctctggagcttcgtctctgctccttcggcccatag

SEQ ID NO: 24 - protein, 221 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGSGATSEQCRELSFFDVVSAGRDPPLELRLCSFGP

SEQ ID NO: 25 - DNA, 708 bp, Zea mays
  source 1..708: mol_type = other DNA
atggacaccgaggcaccatcaggccgcgtcggcgtcaagtgtggaagatggaacccaacaccagagcaagtcaaggtgctcaccgagctcttcagagctggcttgaggacaccatcaactgaccagatccaaaggatctcagctcatctaggtgccttcggcaaggtggagagcaagaatgtcttctactggttccagaaccacaaggcaagagaaaggcatcatcacaagaagaagaagagaaggcgtagaggagcagctatggcatcatcaagctcaccagactccggcagcggctcaggaagctcagaagaagatggccgcgtcgccgccgccgccgacgccgatctagtccttcaaccatcaccaggaagatcaaagagcaagagagaagcaagaagaagctatggaggaggccatcatcgacggctggtgacatgcaagcagctggcccacacccaccggtatgttagagatgtggtggagcagcaagaagaagacacctgggagaggccaacaagagaagccgtggaaacattggagctcttccctctcaagagctatgttgacctagaagctgagaaggtgagaaggtatgtggtgagggccggcgcctccgagcaatgtcgagacttctccttcttcgatgtctcaggaggccgagatcctcctcttcttgagcttcgtctctgctccttcggcccatag

SEQ ID NO: 26 - protein, 235 aa, Zea mays
MDTEAPSGRVGVKCGRWNPTPEQVKVLTELFRAGLRTPSTDQIQRISAHLGAFGKVESKNVFYWFQNHKARERHHHKKKKRRRRGAAMASSSSPDSGSGSGSSEEDGRVAAAADADLVLQPSPGRSKSKREARRSYGGGHHRRLVTCKQLAHTHRYVRDVVEQQEEDTWERPTREAVETLELFPLKSYVDLEAEKVRRYVVRAGASEQCRDFSFFDVSGGRDPPLLELRLCSFGP

SEQ ID NO: 27 - DNA, 549 bp, Arabidopsis thaliana
  source 1..549: mol_type = other DNA
atgagcttctccgtcaagggaagaagcctccgaggcaacaacaacggcggcaccggcaccaagtgtggaaggtggaaccccaccgtggagcagctcaagatcctcaccgacctcttcagggccggcctcaggacccccaccaccgaccagatccagaagatctccaccgagctgagcttctacggcaagattgagagcaagaacgtcttctactggttccagaaccacaaggcaagagaacggcagaagagaaggaagatcagcatcgacttcgaccatcatcatcatcaaccatccacccgtgatgtcttcgagatctccgaggaggattgtcaagaagaagagaaggtgatcgagactctccagctcttccccgtcaacagcttcgaggacagcaacagcaaggtggacaagatgagggcaagaggcaacaaccagtaccgagaatatatccgtgagacaaccaccacctccttctcaccctacagcagctgcggcgccgagatggagcatcctcctcctctggacctccgcctctccttcctctga

SEQ ID NO: 28 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 29 - protein, 67 aa, synthetic construct
VKCGRWNPTAEQVKVLTELFRAGLRTPSTEQIQRISTHLSAFGKVESKNVFYWFQNHKARERHHHKK

SEQ ID NO: 30 - DNA, 45 bp, synthetic construct
  source 1..45: mol_type = other DNA
taatacgactcactatagccctctttccccaacctcgtgttgttc

SEQ ID NO: 31 - DNA, 34 bp, synthetic construct
  source 1..34: mol_type = other DNA
cgcacacacaacacaaccggtccattattaaacc

SEQ ID NO: 32 - protein, 8 aa, synthetic construct
  VARIANT 3: note = X is Asp, Glu, Gln or Pro
  VARIANT 7: note = X is Gly, Ile, Thr, Val or Leu
  VARIANT 8: note = X is Gly, Ser, Lys, Asn, Thr, Cys, Val or Leu
TLXLFPXX

SEQ ID NO: 33 - protein, 5 aa, synthetic construct
  VARIANT 2: note = X is Glu or Asp
LXLRL

SEQ ID NO: 34 - DNA, 28 bp, synthetic construct
  source 1..28: mol_type = other DNA
tttgcgaactggtattccaaggaggcgg

SEQ ID NO: 35 - DNA, 65 bp, Triticum aestivum
  source 1..65: mol_type = other DNA
atgagaacctggaagaaatgggagacagagaccgcctccctggaataccagttcgcaaatggtca

SEQ ID NO: 36 - DNA, 65 bp, Triticum aestivum; identical to SEQ ID NO: 35
  source 1..65: mol_type = other DNA

SEQ ID NO: 37 - DNA, 65 bp, Triticum aestivum
  source 1..65: mol_type = other DNA
atgaggacctggaagaaatgggagacagagaccgcctccctggaataccagttcgcaaatggtca

SEQ ID NO: 38 - DNA, 201 bp, synthetic construct
  source 1..201: mol_type = other DNA
tccttaacctgctccggcataatattcttattcctccccccggcagatgcagacatggaagaaatgggagacagagaccgcctccttggaataccagttcgcaaatggtcagacaattttccaaatgaaacctcttctgttttgatgcgtttacagaggcaggcatgatcagagcgagtgaactgatgatatgttcttctc

SEQ ID NO: 39 - protein, 9 aa, synthetic construct
LAGLIDADG

SEQ ID NO: 40 - protein, 5 aa, synthetic construct
  VARIANT 4: note = X is Arg, Ser or Thr
LELXL